(19) United States
(12) Patent Application Publication
(10) Pub. No.: US 2006/0252818 A1
(43) Pub. Date: Nov. 9, 2006

(54) Monoamine re-uptake inhibitors and methods relating thereto

(75) Inventors: Mehrak Kiankarimi, Carlsbad, CA (US); Sarah Hudson, San Diego, CA (US); Wesley J. Dwight, Poway, CA (US); Warren S. Wade, San Diego, CA (US)

Correspondence Address: NEUROCRINE BIOSCIENCES, INC., 12790 EL CAMINO REAL, SAN DIEGO, CA, 92130, US

(73) Assignee: Neurocrine Biosciences, Inc.

(21) Appl. No.: 11/429,018

(22) Filed: May 5, 2006

Related U.S. Application Data
(60) Provisional application No. 60/678,663, filed on May 6, 2005.
(60) Provisional application No. 60/734,994, filed on Nov. 8, 2005.

(51) Int. Cl. A61K 31/382; A61K 31/353; C07D 335/06

(52) U.S. Cl. 514/432; 514/456; 549/23; 549/403

(57) ABSTRACT

Monoamine re-uptake inhibitors and more specifically serotonin and noradrenaline re-uptake inhibitors are disclosed that have utility in the treatment of disorders of the central or peripheral nervous system in both men and women. The compounds of this invention have the structure: 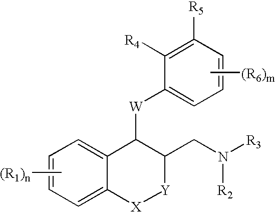
wherein R₁, R₂, R₃, R₄, R₅, R₆, m, n, W, X, and Y are as defined herein, including stereoisomers, prodrugs and pharmaceutically acceptable salts, esters and solvates thereof. Also disclosed are compositions containing a compound of this invention in combination with a pharmaceutically acceptable carrier, as well as methods relating to the use thereof for inhibiting monoamine re-uptake in a subject in need thereof.

### CROSS-REFERENCE TO RELATED APPLICATIONS

This application claims the benefit of U.S. Provisional Patent Application No. 60/678,663 filed May 6, 2005 and U.S. Provisional Patent Application No. 60/734,994 filed Nov. 8, 2005, which applications are incorporated herein by reference in their entirety.

### FIELD OF THE INVENTION

This invention relates generally to monoamine re-uptake inhibitors and more specifically to serotonin and noradrenaline re-uptake inhibitors, and to methods of treating disorders by administration of such inhibitors to a warm-blooded animal in need thereof.

### BACKGROUND OF THE INVENTION

Decreased concentrations of monoamine neurotransmitters, such as serotonin (also known as 5-hydroxytryptamine or 5-HT), noradrenaline (norepinephrine), and dopamine, are implicated in a number of disorders of the central or peripheral nervous system. These disorders include depression, eating disorders, schizophrenia, inflammatory bowel disorders, pain, addiction disorders, urinary incontinence, dementia, Alzheimer's, memory loss, Parkinsonism, anxiety, attention-deficit disorder, social phobia, obsessive compulsive disorder, substance abuse and withdrawal, cognitive disorders, fibromyalgia and sleep disorders. These neurotransmitters travel from the terminal of a neuron across a small gap (i.e., the synaptic cleft) and bind to receptor molecules on the surface of a second neuron. This binding elicits intracellular changes that initiate or activate a response or change in the postsynaptic neuron. Inactivation occurs primarily by transport (i.e., reuptake) of the neurotransmitter back into the presynaptic neuron. Enhancing the amount of one or more of these monoamines has been shown to have utility in the treatment of disorders such as depression, anxiety, neuropathic pain, fibromyalgia, urinary incontinence and attention deficit hyperactivity disorder (ADHD). One advantageous method to increase the amount of a monoamine or monoamines is by administering a re-uptake inhibitor which has a particular selectivity/affinity to one or more monoamine transporters.

Selective serotonin re-uptake inhibitors (SSRIs) function by inhibiting the reuptake of serotonin by afferent neurons. SSRIs well known in the art include sertraline (Zoloft®), fluoxetine (Prozac®) and paroxetine (Paxil®). Selective noradrenaline (or norepinephrine) re-uptake inhibitors function by increasing noradrenaline levels and include drugs known in the art including reboxetine (Edronax®), atomoxetine (Strattera®), and buproprion (Wellbutrin®). Dual serotonin-noradrenaline re-uptake inhibitors (SNRIs) which inhibit the reuptake of both serotonin and norepinephrine include venlafaxine (Effexor®), duloxetine (Cymbalta®), milnacipran and imipramine (Tofranil®).

While significant strides have been made in this field, there remains a need in the art for effective small molecule monoamine re-uptake inhibitors. These inhibitors may advantageously possess characteristics such as enhanced selectively toward one or more monoamine transporters, enhanced pharmacokinetic properties (such as half-life, bioavailability, and minimal interaction with liver enzymes such as the cytochrome P450 family), and/or enhanced potency. There is also a need for pharmaceutical compositions containing such monoamine re-uptake inhibitors, as well as methods relating to the use thereof to treat, for example, conditions caused by low concentrations of a monoamine or monoamines. The present invention fulfills these needs, and provides other related advantages.

### BRIEF SUMMARY OF THE INVENTION

In brief, this invention is generally directed to monoamine re-uptake inhibitors, in particular, serotonin and/or noradrenaline reuptake inhibitors, as well as to methods for their preparation and use, and to pharmaceutical compositions containing the same. More specifically, the monoamine re-uptake inhibitors of this invention are compounds having the following general structure (I): 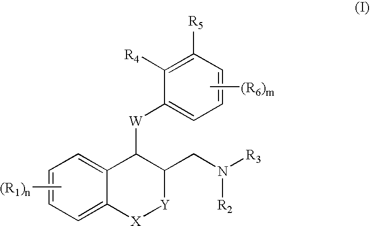
including stereoisomers, prodrugs and pharmaceutically acceptable salts, esters and solvates thereof, wherein R₁, R₂, R₃, R₄, R₅, R₆, m, n, W, X, and Y are as defined below.

The monoamine reuptake inhibitors of this invention may have utility over a wide range of therapeutic applications, and may be used to treat a variety of disorders of the central or peripheral nervous system in both men and women, as well as a mammal in general (also referred to herein as a “subject”). For example, such conditions include, but are not limited to, depression, eating disorders, schizophrenia, inflammatory bowel disorders, pain, addiction disorders, urinary incontinence, dementia, Alzheimer's, memory loss, Parkinsonism, anxiety, attention-deficit disorder, social phobia, obsessive compulsive disorder, substance abuse and withdrawal, cognitive disorders, fibromyalgia and sleep disorders. Conditions of particular interest which may be treated by administration of compounds of structure (I) include depression, anxiety, neuropathic pain, fibromyalgia, urinary incontinence and attention deficit hyperactivity disorder (ADHD). The compounds may also be useful in combination with antipsychotic agents for the treatment of schizophrenia, as well as in combination with dopaminergic agents for use in Parkinson's disease.

The methods of this invention include administering an effective amount of a monoamine re-uptake inhibitor, preferably in the form of a pharmaceutical composition, to a mammal in need thereof. Thus, in still a further embodiment, pharmaceutical compositions are disclosed containing one or more monoamine re-uptake inhibitors of this invention in combination with a pharmaceutically acceptable carrier and/or diluent.

These and other aspects of the invention will be apparent upon reference to the following detailed description. To this end, various references are set forth herein which describe in more detail certain background information, procedures, compounds and/or compositions, and are each hereby incorporated by reference in their entirety.

### DETAILED DESCRIPTION OF THE INVENTION

As mentioned above, the present invention is directed generally to compounds useful as monoamine reuptake inhibitors. The compounds of this invention have the following structure (I): 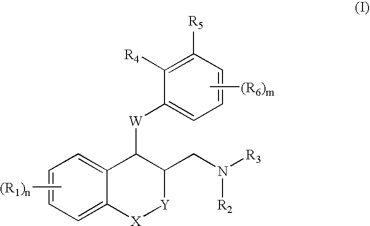
and stereoisomers, prodrugs and pharmaceutically acceptable salts, esters and solvates thereof,

wherein:

W is O or S;

X—Y is —O—, —OCH₂—, —CH₂O—, —SCH₂—, or —CH₂S—;

R₁ at each occurrence is independently halo, CN, CF₃, OH, lower alkyl, lower alkoxy, or lower thioalkyl;

R₂, R₃ are independently H, lower alkyl, or substituted lower alkyl;

R₄, R₅ are independently H, halo, CN, CF₃, OH, NO₂, lower alkyl, substituted lower alkyl, lower alkoxy, substituted lower alkoxy, lower thioalkyl, or substituted lower thioalkyl;

or R₄ and the carbon to which it is attached taken together with R₅ and the carbon to which it is attached form a 5-6 member carbocycle or a 5-6 member heterocycle where the carbocycle or heterocycle is substituted with 0-4 R₇;

R₆, R₇ are at each occurrence independently H, halo, CN, CF₃, OH, lower alkyl, substituted lower alkyl, lower alkoxy, or lower thioalkyl;

m is 0, 1, 2, or 3; and

n is 0, 1, 2 or 3.

As used herein, the above terms have the following meaning:

“Alkyl” means a straight chain or branched, noncyclic or cyclic, unsaturated or saturated aliphatic hydrocarbon containing from 1 to 10 carbon atoms, while the term “lower alkyl” has the same meaning as alkyl but contains from 1 to 4 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Representative saturated cyclic alkyls include cyclopropyl, cyclobutyl, cyclopentyl, cyclohexyl, —CH₂-cyclopropyl, —CH₂-cyclobutyl, —CH₂-cyclopentyl, —CH₂-cyclohexyl, and the like; while unsaturated cyclic alkyls include cyclopentenyl and cyclohexenyl, and the like. Cyclic alkyls include di- and poly-homocyclic rings such as decalin and adamantyl. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

“Aryl” means an aromatic carbocyclic moiety such as phenyl or naphthyl.

“5-6 Member carbocycle” means a ring composed of 5 or 6 carbon atoms, either saturated, unsaturated or aromatic.

“Heteroaryl” means an aromatic heterocycle ring of 5- to 10-members and having at least one heteroatom selected from nitrogen, oxygen and sulfur, and containing at least 1 carbon atom, including both mono- and bicyclic ring systems. Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

“Heterocycle” (also referred to herein as a “heterocycle ring”) means a 5- to 7-membered monocyclic, or 7- to 14-membered polycyclic, heterocycle ring which is either saturated, unsaturated or aromatic, and which contains from 1 to 4 heteroatoms independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring as well as tricyclic (and higher) heterocyclic rings. The heterocycle may be attached via any heteroatom or carbon atom. Heterocycles include heteroaryls as defined above. Thus, in addition to the aromatic heteroaryls listed above, heterocycles also include (but are not limited to) morpholinyl, pyrrolidinonyl, pyrrolidinyl, piperizinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Halogen” or “halo” means fluoro, chloro, bromo and iodo.

“Alkoxy” means an alkyl moiety attached through an oxygen bridge (i.e., —O-alkyl), such as —O-methyl, —O-ethyl, and the like.

“Thioalkyl” means an alkyl moiety attached through a sulfur bridge (i.e., —S-alkyl) such as —S-methyl, —S-ethyl, and the like.

Lastly, the term “substituted” as used herein means any of the above groups (i.e., alkyl, carbocycle, aryl, or heterocycle) wherein at least one hydrogen atom is replaced with a substituent. In the case of an oxo substituent (“═O”) two hydrogen atoms are replaced. “Substituents” within the context of this invention include halogen, hydroxy, oxo, cyano, nitro, amino, alkylamino, dialkylamino, alkyl, alkoxy, thioalkyl, haloalkyl, hydroxyalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NRₐR_{b}, —NRₐC(═O)OR_{b} —NRₐSO₂R_{b}, —ORₐ, —C(═O)Rₐ —C(═O)ORₐ, —C(═O)NRₐR_{b}, —OC(═O)NRₐR_{b}, —SH, —SRₐ, —SORₐ, —S(═O)₂Rₐ, —OS(═O)₂Rₐ, —S(═O)₂ORₐ, wherein Rₐ and R_{b} are the same or different and independently hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

In an embodiment of the present invention, —X—Y— of structure (I) is —O—, —OCH₂— and —CH₂—O—, as shown in structure (II), (III), and (IV), respectively.

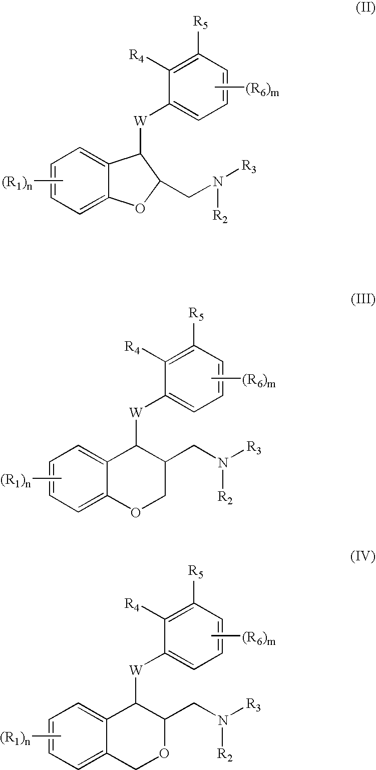

In another embodiment, —X—Y— of structure (I) is —SCH₂— and —CH₂S— as shown in structures (V) and (VI) respectively.

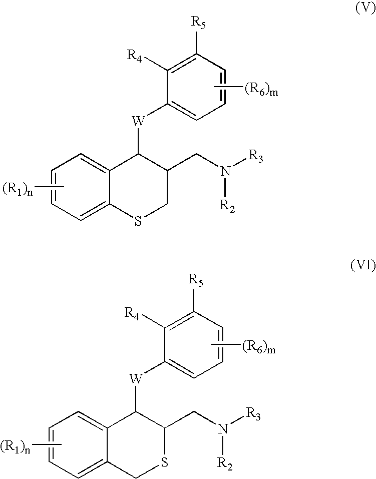

In another embodiment, R₄ and R₅ of structure (I) taken together with the carbons to which they are attached form a ring such as thiophene creating a benzothiophen-7-yl group as shown in structure (VII). In an alternate embodiment, R₄ and R₅ do not cyclize and are represented as methyl and fluoro respectively, as shown in structure (VIII).

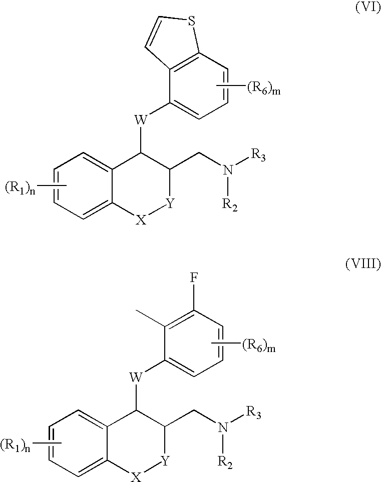

In another embodiment, the compounds of the present invention exist as a mix of 4 diastereomers as shown in Structure (IX) when no stereochemistry is shown. A structure such as structure (X) which shows stereochemistry and includes the term “racemic” is intended to encompass the 2 enantiomers where the stereocenters are either both pointing into or out of the plane (a ‘cis’ configuration).

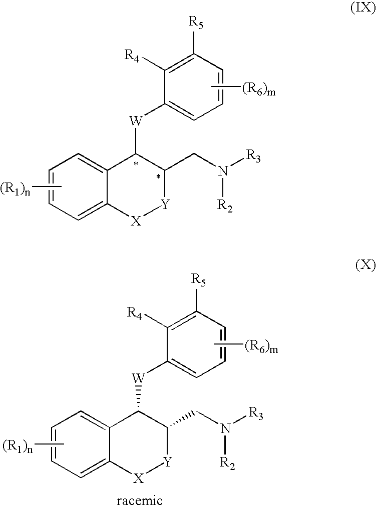

In another embodiment, Structure (XI) is labeled “racemic” and is meant to include the 2 ‘trans’ enantiomers shown as structures (XII) and (XIII).

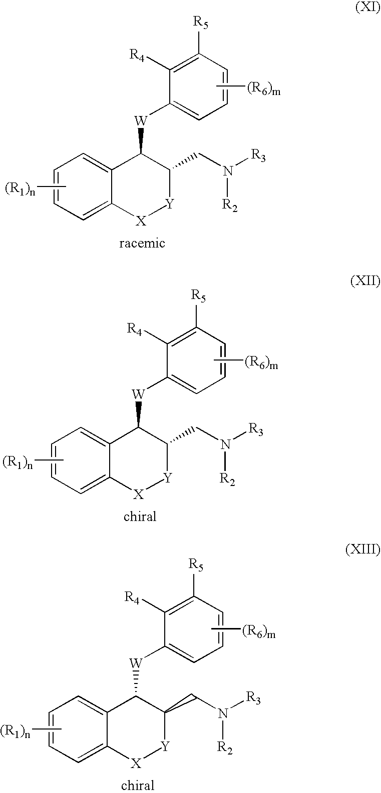

In another embodiment of the invention W is O.

In another embodiment X—Y is —OCH₂— or —CH₂O—.

In an embodiment R₁ is absent or is halo.

In another embodiment R₂ is H and R₃ is lower alkyl.

In an embodiment R₄ is lower alkyl or halo where lower alkyl is methyl or ethyl.

In another embodiment R₅ is H or halo.

In an embodiment R₄ and the carbon to which it is attached taken together with R₅ and the carbon to which it is attached form a 5-6 member heterocycle where the heterocycle is substituted with 0-4 R₇ and the heteroatoms of the ring are O or S.

In another embodiment R₆ is H or halo.

In another embodiment, the term ‘substituted’ as used with substituted lower alkyl, substituted lower alkoxy and substituted lower thioalkyl means replacing a hydrogen from the lower alkyl, lower alkoxy or lower thioalkyl with a substituent selected from halo, alkoxy, amino, alkylamino, dialkylamino, hydroxyl, or cyano.

Particular individual compounds of the present invention include:
[(2R,3R and 2S,3R)-3-(4-Chloro-phenoxy)-2,3-dihydro-benzofuran-2-ylmethyl]-methyl-amine (example 1-1);
[(3R,4R and 3S,4S)-4-(4-Chloro-phenoxy)-chroman-3-ylmethyl]-methyl-amine (2-1);
Methyl-((3R,4R and 3S,4S)-4-phenoxy-chroman-3-ylmethyl)-amine (2-2);
[(3R,4R and 3S,4S)-4-(3-Chloro-phenoxy)-chroman-3-ylmethyl]-methyl-amine (2-3);
[(3R,4R and 3S,4S)-4-(3,4-Dichloro-phenoxy)-chroman-3-ylmethyl]-methyl-amine (3-1);
[(3R,4R and 3S,4S)-4-(2-Chloro-4-methyl-phenoxy)-chroman-3-ylmethyl]-methyl-amine (4-1);
[(3R,4R and 3S,4S)-4-(4-Bromo-2-methyl-phenoxy)-chroman-3-ylmethyl]-methyl-amine (4-2);
[(3R,4R and 3S,4S)-4-(4-Chloro-2-methyl-phenoxy)-chroman-3-ylmethyl]-methyl-amine (4-3);
Methyl-[(3R,4R and 3S,4S)-4-(naphthalen-1-yloxy)-chroman-3-ylmethyl]-amine (4-4);
Methyl-[(3R,4R and 3S,4S)-4-(2-methyl-4-nitro-phenoxy)-chroman-3-ylmethyl]-amine (4-5);
[(3R,4R and 3S,4S)-4-(2-Bromo-phenoxy)-chroman-3-ylmethyl]-methyl-amine (4-6);
[(3R,4R and 3S,4S)-4-(2-Chloro-phenoxy)-chroman-3-ylmethyl]-methyl-amine (4-7);
[(3R,4R and 3S,4S)-4-(3-Bromo-2-methyl-phenoxy)-chroman-3-ylmethyl]-methyl-amine (4-8);
[(3R,4R and 3S,4S)-4-(3-Fluoro-2-methyl-phenoxy)-chroman-3-ylmethyl]-methyl-amine (4-9);
[(3R,4R and 3S,4S)-4-(2-Fluoro-phenoxy)-chroman-3-ylmethyl]-methyl-amine (4-10);
Methyl-[(3R,4R and 3S,4S)-4-(2-vinyl-phenoxy)-chroman-3-ylmethyl]-amine (4-11);
[(3R,4R)-4-(2-Chloro-phenoxy)-chroman-3-ylmethyl]-methyl-amine (4-12);
[(3S,4S)-4-(2-Chloro-phenoxy)-chroman-3-ylmethyl]-methyl-amine (4-13);
[(3R,4S and 3S,4R)-4-(3-Fluoro-2-methyl-phenoxy)-chroman-3-ylmethyl]-methyl-amine (4-14);
2-((3R,4R and 3S,4S)-3-Methylaminomethyl-chroman-4-yloxy)-benzonitrile (5-1);
Methyl-[(3R,4R)-4-(naphthalen-1-yloxy)-chroman-3-ylmethyl]-amine (6-1);
Methyl-[(3S,4S)-4-(naphthalen-1-yloxy)-chroman-3-ylmethyl]-amine (6-2);
[(3S,4S)-4-(3-Fluoro-2-methyl-phenoxy)-chroman-3-ylmethyl]-methyl-amine (6-3);
[(3R,4R)-4-(3-Fluoro-2-methyl-phenoxy)-chroman-3-ylmethyl]-methyl-amine (6-4);
Methyl-[(3R,4R and 3S,4S)-4-(2-trifluoromethyl-phenoxy)-chroman-3-ylmethyl]-amine (6-5);
Methyl-[(3R,4R)-4-(2-trifluoromethyl-phenoxy)-chroman-3-ylmethyl]-amine (6-6);
Methyl-[(3S,4S)-4-(2-trifluoromethyl-phenoxy)-chroman-3-ylmethyl]-amine (6-7);
Dimethyl-((3R,4R and 3S,4S)-4-o-tolyloxy-chroman-3-ylmethyl)-amine (7-1);
Methyl-((3R,4R and 3S,4S)-4-o-tolyloxy-chroman-3-ylmethyl)-amine (7-2);
Methyl-((3R,4R)-4-o-tolyloxy-chroman-3-ylmethyl)-amine (7-3);
Methyl-((3S,4S)-4-o-tolyloxy-chroman-3-ylmethyl)-amine (7-4);
[(3R,4R and 3S,4S)-4-(4-Fluoro-2-methyl-phenoxy)-chroman-3-ylmethyl]-methyl-amine (7-5);
[(3R,4R and 3S,4S)-4-(4-Fluoro-2-methyl-phenoxy)-isochroman-3-ylmethyl]-methyl-amine (7-6);
[(3R,4R and 3S,4S)-4-(6-Fluoro-2-methyl-phenoxy)-isochroman-3-ylmethyl]-methyl-amine (7-7);
[(3S,4S)-4-(6-Fluoro-2-methyl-phenoxy)-isochroman-3-ylmethyl]-methyl-amine (7-8);
[(3R,4R)-4-(6-Fluoro-2-methyl-phenoxy)-isochroman-3-ylmethyl]-methyl-amine (7-9);
[(3R,4R and 3S,4S)-4-(5-Fluoro-2-methyl-phenoxy)-isochroman-3-ylmethyl]-methyl-amine (7-8);
[(3R,4R and 3S,4S)-4-(3-Fluoro-4-methyl-phenoxy)-isochroman-3-ylmethyl]-methyl-amine (7-8);
Methyl-((3R,4S and 3S,4R)-4-o-tolyloxy-chroman-3-ylmethyl)-amine (9-1);
Methyl-((3S,4R)-4-o-tolyloxy-chroman-3-ylmethyl)-amine (9-2);
Methyl-((3R,4S)-4-o-tolyloxy-chroman-3-ylmethyl)-amine (9-3);
Dimethyl-((3R,4R and 3S,4S)-4-o-tolyloxy-thiochroman-3-ylmethyl)-amine (10-1);
Methyl-((3R,4R and 3S,4S)-4-o-tolyloxy-thiochroman-3-ylmethyl)-amine (10-2);
((3R,4S and 3S,4R)-6-Fluoro-4-o-tolyloxy-chroman-3-ylmethyl)-methyl-amine (11-1);
Methyl-((3R,4R and 3S,4S)-4-o-tolyloxy-isochroman-3-ylmethyl)-amine (12-1);
Methyl-((3S,4S)-4-o-tolyloxy-isochroman-3-ylmethyl)-amine (12-2);
Methyl-((3R,4R)-4-o-tolyloxy-isochroman-3-ylmethyl)-amine (12-3);
Methyl-[(3R,4R and 3S,4S)-4-(2-trifluoromethyl-phenoxy)-isochroman-3-ylmethyl]-amine (12-4);
Methyl-[(3R,4R)-4-(2-trifluoromethyl-phenoxy)-isochroman-3-ylmethyl]-amine (12-5);
Methyl-[(3S,4S)-4-(2-trifluoromethyl-phenoxy)-isochroman-3-ylmethyl]-amine (12-6);
Methyl-[(3S,4S)-4-(3-fluoro-2-methyl-phenoxy)-isochroman-3-ylmethyl]-amine (12-7);
Methyl-[(3S,4S)-4-(2-bromo-phenoxy)-isochroman-3-ylmethyl]-amine (12-8);
Methyl-[(3R,4R)-4-(2-bromo-phenoxy)-isochroman-3-ylmethyl]-amine (12-9);
[(3R,4R and 3S,4S)-4-(2-Fluoro-6-trifluoromethyl-phenoxy)-isochroman-3-ylmethyl]-methyl-amine (12-10);
[(3R,4R and 3S,4S)-4-(2-Fluoro-phenoxy)-isochroman-3-ylmethyl]-methyl-amine (12-11);
[(3R,4R)-4-(2-Fluoro-6-trifluoromethyl-phenoxy)-isochroman-3-ylmethyl]-methyl-amine (12-12);
[(3S,4S)-4-(2-Fluoro-6-trifluoromethyl-phenoxy)-isochroman-3-ylmethyl]-methyl-amine (12-13);
Dimethyl-((3S,4R and 3R,4S)-4-o-tolyloxy-isochroman-3-ylmethyl)-amine (13-1);
Methyl-((3S,4R and 3R,4S)-4-o-tolyloxy-isochroman-3-ylmethyl)-amine (13-2);
Methyl-((3R,4S)-4-o-tolyloxy-isochroman-3-ylmethyl)-amine (13-3);
Methyl-((3S,4R)-4-o-tolyloxy-isochroman-3-ylmethyl)-amine (13-4);
Methyl-[(3R,4S and 3S,4R)-4-(naphthalen-1-yloxy)-isochroman-3-ylmethyl]-amine (14-1);
[(3R,4S and 3S,4R)-4-(2-Methoxy-phenoxy)-isochroman-3-ylmethyl]-methyl-amine (14-2);
[(3R,4S and 3S,4R)-4-(2-Ethoxy-phenoxy)-isochroman-3-ylmethyl]-methyl-amine (14-3);
[(3R,4S and 3S,4R)-4-(2,2-Dimethyl-2,3-dihydro-benzofuran-7-yloxy)-isochroman-3-ylmethyl]-methyl-amine (14-4);
[(3R,4S and 3S,4R)-4-(Benzofuran-7-yloxy)-isochroman-3-ylmethyl]-methyl-amine (14-5);
[(3R,4S and 3S,4R)-4-(Benzofuran-4-yloxy)-isochroman-3-ylmethyl]-methyl-amine (14-6);
[(3R,4S and 3S,4R)-4-(Benzo[b]thiophen-7-yloxy)-isochroman-3-ylmethyl]-methyl-amine (14-7);
[(3R,4S and 3S,4R)-4-(Benzo[b]thiophen-4-yloxy)-isochroman-3-ylmethyl]-methyl-amine (14-8);
Methyl-[(3R,4S and 3S,4R)-4-(2-methylsulfanyl-phenoxy)-isochroman-3-ylmethyl]-amine (14-9);
[(3R,4S)-4-(Benzo[b]thiophen-7-yloxy)-isochroman-3-ylmethyl]-methyl-amine (14-10);
[3S,4R)-4-(Benzo[b]thiophen-7-yloxy)-isochroman-3-ylmethyl]-methyl-amine (14-11);
[(3S,4R)-4-(Benzo[b]thiophen-4-yloxy)-isochroman-3-ylmethyl]-methyl-amine (14-12);
[(3R,4S)-4-(Benzo[b]thiophen-4-yloxy)-isochroman-3-ylmethyl]-methyl-amine (14-13);
[(3R,4S and 3S,4R)-4-(2,4-Dimethyl-phenoxy)-isochroman-3-ylmethyl]-methyl-amine (14-14);
[(3R,4S)-4-(2,4-Dimethyl-phenoxy)-isochroman-3-ylmethyl]-methyl-amine (14-15);
[(3S,4R )-4-(2,4-Dimethyl-phenoxy)-isochroman-3-ylmethyl]-methyl-amine (14-16);
[(3R,4R and 3S,4S)-4-(2,3-Dimethyl-phenoxy)-chroman-3-ylmethyl]-methyl-amine (15-1);
[(3R,4R)-4-(2,3-Dimethyl-phenoxy)-chroman-3-ylmethyl]-methyl-amine (15-2);
[(3S,4S)-4-(2,3-Dimethyl-phenoxy)-chroman-3-ylmethyl]-methyl-amine (15-3);
[(3R,4R and 3S,4S)-4-(2,4-Dimethyl-phenoxy)-chroman-3-ylmethyl]-methyl-amine (15-4);
[(3R,4R and 3S,4S)-4-(2-Ethyl-phenoxy)-chroman-3-ylmethyl]-methyl-amine (16-1);
[(3R,4R)-4-(2-Ethyl-phenoxy)-chroman-3-ylmethyl]-methyl-amine (16-2);
[(3S,4S)-4-(2-Ethyl-phenoxy)-chroman-3-ylmethyl]-methyl-amine (16-3);
[(3R,4R and 3S,4S)-4-(4-Hydroxy-2-methyl-phenoxy)-chroman-3-ylmethyl]-methyl-amine (17-1);
[(3R,4R)-4-(4-Hydroxy-2-methyl-phenoxy)-chroman-3-ylmethyl]-methyl-amine (17-2);
[(3S,4S)-4-(4-Hydroxy-2-methyl-phenoxy)-chroman-3-ylmethyl]-methyl-amine (17-3);
C-((3R,4R and 3S,4S)-4-o-Tolyloxy-chroman-3-yl)-methylamine (18-1);
C-((3S,4S)-4-o-Tolyloxy-chroman-3-yl)-methylamine (18-2);
C-((3R,4R)-4-o-Tolyloxy-chroman-3-yl)-methylamine (18-3);
C-((3R,4R and 3S,4S)-4-o-Tolyloxy-isochroman-3-yl)-methylamine (18-4);
C-((3R,4R)-4-o-Tolyloxy-isochroman-3-yl)-methylamine (18-5);
C-((3S,4S)-4-o-Tolyloxy-isochroman-3-yl)-methylamine (18-6);
[(3R,4R and 3S,4S)-4-(2-Methoxymethyl-phenoxy)-chroman-3-ylmethyl]-methyl-amine (19-1);
[(3R,4R and 3S,4S)-4-(2-Ethyl-phenoxy)-isochroman-3-ylmethyl]-methyl-amine (20-1);
[(3R,4S and 3S,4R)-4-(2-Ethyl-phenoxy)-isochroman-3-ylmethyl]-methyl-amine (20-2);
[(3R,4R and 3S,4S)-4-(3-Fluoro-2-methyl-phenoxy)-isochroman-3-ylmethyl]-methyl-amine (20-3);
[(3R,4S and 3S,4R)-4-(3-Fluoro-2-methyl-phenoxy)-isochroman-3-ylmethyl]-methyl-amine (20-4);
[(3R,4R and 3S,4S)-4-(2-Chloro-phenoxy)-isochroman-3-ylmethyl]-methyl-amine (20-5);
[(3R,4R)-4-(2-Chloro-phenoxy)-isochroman-3-ylmethyl]-methyl-amine (20-6);
[(3S,4S)-4-(2-Chloro-phenoxy)-isochroman-3-ylmethyl]-methyl-amine (20-7);
[(3R,4S and 3S,4R)-4-(2-Chloro-phenoxy)-isochroman-3-ylmethyl]-methyl-amine (20-8);
[(3R,4S and 3S,4R)-4-(3-Chloro-2-methyl-phenoxy)-isochroman-3-ylmethyl]-methyl-amine (20-9);
Methyl-[(3R,4R and 3S,4S)-4-(2-methylsulfanyl-phenoxy)-isochroman-3-ylmethyl]-amine (21-1);
Methyl-[(3R,4R and 3S,4S)-4-(2-methylsulfanyl-phenoxy)-chroman-3-ylmethyl]-amine (21-2);
[(3R,4R and 3S,4S)-4-(2-Chloro-phenoxy)-thiochroman-3-ylmethyl]-methyl-amine (23-1);
[(3R,4R and 3S,4S)-4-(2-Chloro-phenoxy)-isothiochroman-3-ylmethyl]-methyl-amine (24-1);
[(3R,4R and 3S,4S)-4-(2-Ethyl-6-fluoro-phenoxy)-isochroman-3-ylmethyl]-methyl-amine (25-1);
[(3R,4R and 3S,4S)-4-(4-Hydroxy-2-methyl-phenoxy)-isochroman-3-ylmethyl]-methyl-amine (26-1);
[(3S,4S)-4-(4-Hydroxy-2-methyl-phenoxy)-isochroman-3-ylmethyl]-methyl-amine (26-2); and
[(3R,4R)-4-(4-Hydroxy-2-methyl-phenoxy)-isochroman-3-ylmethyl]-methyl-amine (26-3).

The compounds of the present invention may be prepared by known organic synthesis techniques, including the methods described in more detail in the Examples. In general, the compounds of structure (I) above may be made by the following reaction schemes, wherein all substituents are as defined above unless indicated otherwise.

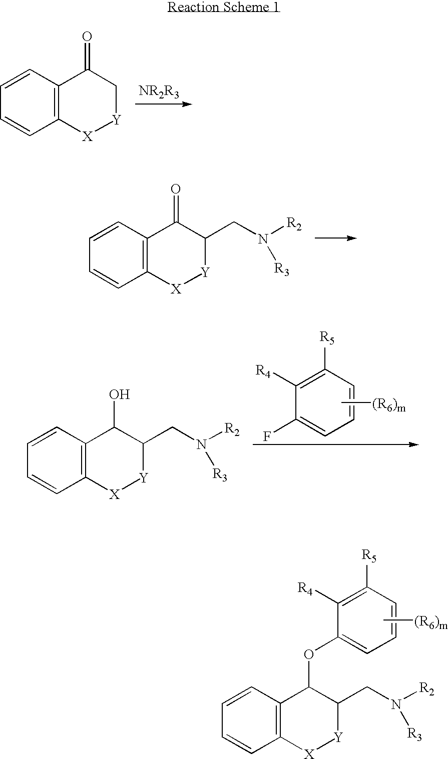

The compounds are typically prepared by Mannich reaction of the cyclic aryl ketone with formaldehyde and a secondary amine, followed by reduction to the alcohol with small reducing agents like sodium borohydride to give predominantly the trans diastereomer or with large reducing agents like L-Selectride to give predominantly the cis diastereomer. A metal salt of the alcohol can then displace a halo aromatic by nucleophilic aromatic substitution to generate a tertiary amine product. The secondary amine is typically produced by reaction of the tertiary amine with a chloroformate followed by decomposition of the resulting carbamate.

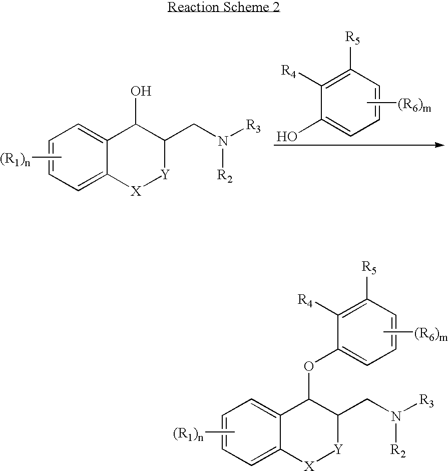

Alternatively, either alcohol diastereomer can be reacted with a phenol under Mitsunobu conditions to generate predominantly the trans product that can be converted to the secondary amine by similar chemistry as above.

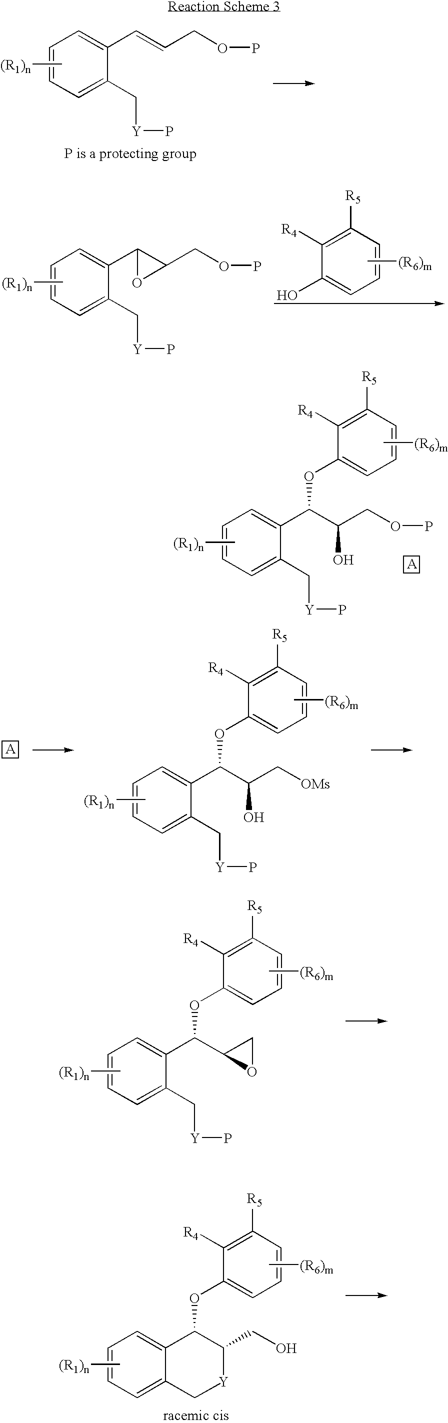

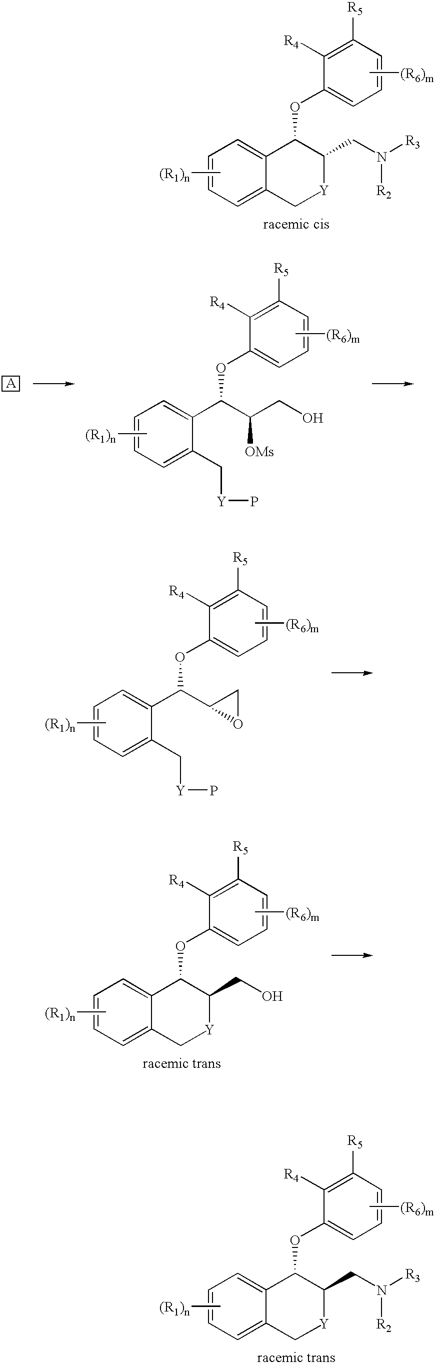

When Y is O, or S, an alternative synthesis is to close the ring late in the sequence. Starting from an optionally protected allyl alcohol with Y protected in a nonnucleophilic form, the double bond can be epoxidized and opened with a phenol. This phenoxy compound is then deprotected if necessary and the resultant diol is activated on the primary alcohol, for example by mesylation, converted to the epoxide, and the ring is formed by deprotection of Y and subsequent opening of the epoxide, generating predominantly the cis isomer. The alternative diastereomer can be accessed by activating the secondary alcohol in the presence of a protected primary alcohol, deprotection and closure to the alternate epoxide. Ring closure in the same manner as the other isomer generates predominantly the trans isomer. The cis and trans primary alcohols can be converted to the amine by activation and displacement with an amine, or by oxidation to the aldehyde followed by reductive amination.

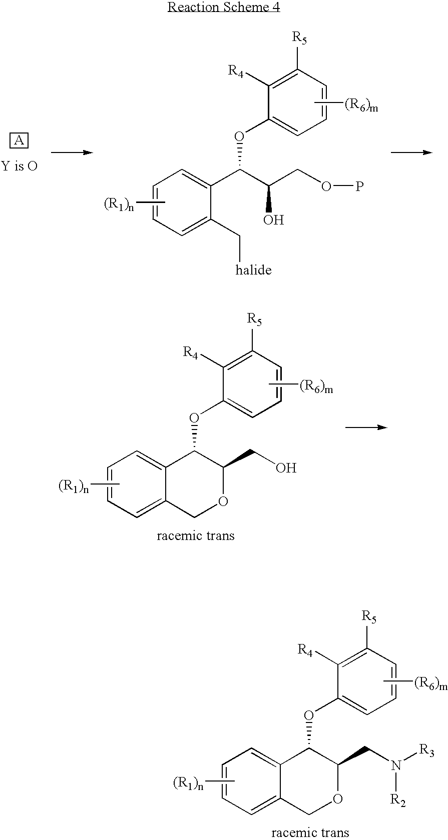

When Y of compound A from scheme 3 is O, the trans diastereomer can also be generated by deprotection and activation of Y for example by conversion to the benzyl halide, followed by nucleophilic attack of the secondary hydroxyl on the activated benzyl position. Other variations in these steps such as opening either diastereomer of the second epoxide with an amine, followed by ring closure on the benzyl position will be readily discernable for one skilled in the art.

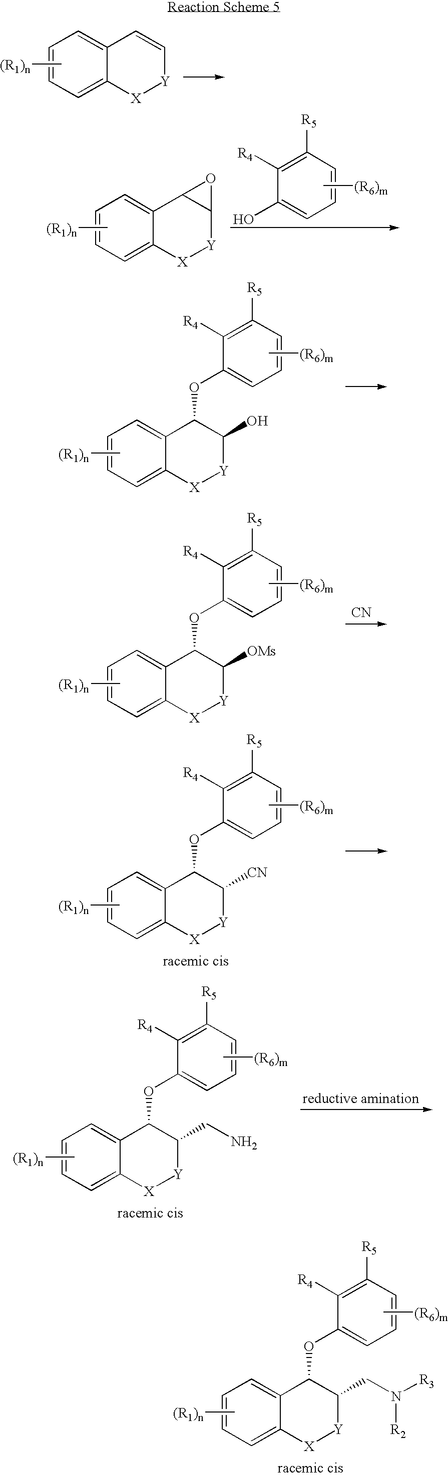

Epoxidation of a cyclic alkene followed by opening with a phenol generates the trans cyclic alcohol. Activation for example as the mesylate and displacement with cyanide can generate predominantly the cis diastereomer, which can be reduced to the primary amine. Reductive amination with aldehydes or ketones generates the substituted products. Alternatively, the amine can be functionalized with alkyl halides such as methyl iodide. For secondary amines, one alternative is to generate the symmetric tertiary amine followed by conversion to the carbamate and decomposition of that product to the desired secondary amine.

The compounds of the present invention may generally be utilized as the free acid or free base. Alternatively, the compounds of this invention may be used in the form of acid or base addition salts. Acid addition salts of the free amino compounds of the present invention may be prepared by methods well known in the art, and may be formed from organic and inorganic acids. Suitable organic acids include maleic, fumaric, benzoic, ascorbic, succinic, methanesulfonic, acetic, trifluoroacetic, oxalic, propionic, tartaric, salicylic, citric, gluconic, lactic, mandelic, cinnamic, aspartic, stearic, palmitic, glycolic, glutamic, and benzenesulfonic acids. Suitable inorganic acids include hydrochloric, hydrobromic, sulfuric, phosphoric, and nitric acids. Base addition salts included those salts that form with the carboxylate anion and include salts formed with organic and inorganic cations such as those chosen from the alkali and alkaline earth metals (for example, lithium, sodium, potassium, magnesium, barium and calcium), as well as the ammonium ion and substituted derivatives thereof (for example, dibenzylammonium, benzylammonium, 2-hydroxyethylammonium, and the like). Thus, the term “pharmaceutically acceptable salt” of structure (I) is intended to encompass any and all acceptable salt forms.

In addition, prodrugs are also included within the context of this invention. Prodrugs are any covalently bonded carriers that release a compound of structure (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol and amine functional groups of the compounds of structure (I). Further, in the case of a carboxylic acid (—COOH), esters may be employed, such as methyl esters, ethyl esters, and the like.

With regard to stereoisomers, the compounds of structure (I) may have chiral centers and may occur as racemates, racemic mixtures and as individual enantiomers or diastereomers. All such isomeric forms are included within the present invention, including mixtures thereof. Furthermore, some of the crystalline forms of the compounds of structure (I) may exist as polymorphs, which are included in the present invention. In addition, some of the compounds of structure (I) may also form solvates with water or other organic solvents. Such solvates are similarly included within the scope of this invention.

As mentioned above, the compounds of this invention and their salts inhibit the uptake of one or more of the monoamine neurotransmitters serotonin, noradrenaline and dopamine. As such, these compounds and their salts may have utility over a wide range of therapeutic applications, and may be used to treat a variety of disorders which are caused by or linked to decreased neurotransmission of one or more of these monoamines. These disorders include disorders of the central and/or peripheral nervous system.

In an embodiment, the compounds of the present invention may selectively inhibit the re-uptake of serotonin and noradrenaline over the re-uptake of dopamine. Other compounds of the present invention may selectively inhibit noradrenaline over both serotonin and dopamine. In another embodiment, compounds of the present invention may selectively inhibit the re-uptake of serotonin over both noradrenaline and dopamine.

In an embodiment, conditions which may be treated by compounds of the current invention include, but are not limited to, depression, eating disorders, schizophrenia, inflammatory bowel disorders, pain, addiction disorders, urinary incontinence, dementia, Alzheimer's, memory loss, Parkinsonism, anxiety, attention-deficit disorder, social phobia, obsessive compulsive disorder, substance abuse and withdrawal, cognitive disorders, fibromyalgia and sleep disorders.

Pain may generally be divided into two categories: acute pain and chronic (or persistent) pain. Acute pain is self-limiting and generally results from injured or diseased tissue and is considered nociceptive in nature. Examples of nociceptive pain include post-operative pain, pain associated with trauma, and the pain of arthritis. Chronic pain can be defined as pain that persists beyond the usual course of the acute injury or disease. Chronic pain is generally neuropathic in nature and can be continuous or recurring. Chronic pain is generally caused by prolonged and sometimes permanent dysfunction of the central or peripheral nervous system. Examples include post herpetic (or post-shingles) neuralgia, reflex sympathetic dystrophy/causalgia (nerve trauma), components of cancer pain, phantom limb pain, entrapment neuropathy (e.g., carpal tunnel syndrome), and peripheral neuropathy (widespread nerve damage due to, for instance, diabetes or excessive alcohol use).

In another embodiment, a compound of structure (I) may be administered along with an antipsychotic to treat schizophrenia. The antipsychotic may be typical or atypical. A compound of structure (I) could also be administered with a dopaminergic agent such as levodopa to treat Parkinson's disease and/or the side effects associated with such therapy.

In another embodiment of the invention, pharmaceutical compositions containing one or more monoamine re-uptake inhibitors are disclosed. For the purposes of administration, the compounds of the present invention may be formulated as pharmaceutical compositions. Pharmaceutical compositions of the present invention comprise a monoamine re-uptake inhibitor of the present invention and a pharmaceutically acceptable carrier and/or diluent. The monoamine re-uptake inhibitor is present in the composition in an amount which is effective to treat a particular disorder—that is, in an amount sufficient to achieve monoamine re-uptake inhibition, and preferably with acceptable toxicity to the patient. Typically, the pharmaceutical compositions of the present invention may include a monoamine re-uptake inhibitor in an amount from 0.1 mg to 250 mg per dosage depending upon the route of administration, and more typically from 1 mg to 60 mg. Appropriate concentrations and dosages can be readily determined by one skilled in the art.

Pharmaceutically acceptable carrier and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated as pills, capsules, granules, or tablets which contain, in addition to a monoamine re-uptake inhibitor, diluents, dispersing and surface active agents, binders, and lubricants. One skilled in this art may further formulate the monoamine re-uptake inhibitor in an appropriate manner, and in accordance with accepted practices, such as those disclosed in Remington's Pharmaceutical Sciences, Gennaro, Ed., Mack Publishing Co., Easton, Pa. 1990.

In another embodiment, the present invention provides a method for treating disorders of the central or peripheral nervous system. Such methods include administering of a compound of the present invention to a warm-blooded animal in an amount sufficient to treat the condition. In this context, “treat” includes prophylactic administration. Such methods include systemic administration of a monoamine re-uptake inhibitor of this invention, preferably in the form of a pharmaceutical composition as discussed above. As used herein, systemic administration includes oral and parenteral methods of administration. For oral administration, suitable pharmaceutical compositions of monoamine re-uptake inhibitors include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parental administration, the compounds of the present invention can be prepared in aqueous injection solutions which may contain, in addition to the monoamine re-uptake inhibitor, buffers, antioxidants, bacteriostats, and other additives commonly employed in such solutions.

The following examples are provided for purposes of illustration, not limitation. In summary, the monoamine re-uptake inhibitors of this invention may be assayed by the methods disclosed in Examples 27 to 31, while the following Examples 1 to 26 disclose the synthesis of representative compounds of this invention.

### EXAMPLES

HPLC Methods for Analyzing the Samples

Retention Time, t_{R}, in Minutes

Analytical HPLC-MS Method 1

Platform: Agilent 1100 series: equipped with an auto-sampler, an UV detector (220 nM and 254 nM), a MS detector (APCI);

HPLC column: Phenomenex Synergi: MAX-RP, 2.0×50 mm column;

HPLC gradient: 1.0 mL/minute, from 10% acetonitrile in water to 90% acetonitrile in water in 2.5 minutes, maintaining 90% for 1 minute. Both acetonitrile and water have 0.025% TFA.

Analytical HPLC-MS Method 2

Platform: Agilent 1100 series: equipped with an auto-sampler, an UV detector (220 nM and 254 nM), a MS detector (APCI);

HPLC column: Phenomenex Synergi-Max RP, 2.0×50 mm column;

HPLC gradient: 1.0 mL/minute, from 5% acetonitrile in water to 95% acetonitrile in water in 13.5 minutes, maintaining 95% for 2 minute. Both acetonitrile and water have 0.025% TFA.

Analytical HPLC-MS Method 3

Platform: Agilent 1100 series: equipped with an auto-sampler, an UV detector (220 nM and 254 nM), a MS detector (electrospray);

HPLC column: XTerra MS, C₁₈, 5 μ, 3.0×250 mm column;

HPLC gradient: 1.0 mL/minute, from 10% acetonitrile in water to 90% acetonitrile in water in 46 minutes, jump to 99% acetonitrile and maintain 99% acetonitrile for 8.04 minutes. Both acetonitrile and water have 0.025% TFA.

Analytical HPLC-MS Method 4

Platform: Agilent 1100 series: equipped with an auto-sampler, an UV detector (220 nM and 254 nM), a MS detector (APCI) and Berger FCM 1200 CO₂ pump module;

HPLC column: Berger Pyridine, PYR 60 A, 6 μ, 4.6×150 mm column;

HPLC gradient: 4.0 mL/minute, 120 bar; from 10% methanol in supercritical CO₂ to 60% methanol in supercritical CO₂ in 1.67 minutes, maintaining 60% for 1 minute. Methanol has 1.5% water. Backpressure regulated at 140 bar.

Analytical HPLC-MS Method 5

Platform: Gilson 215 Auto-sampler, Dionex Thermostatted Column Compartment TCC-100 held at 30° C., Dionex PDA-100 Photodiode Array Detector (220 nm and 254 nm), Dionex P680 HPLC pump, Thermo Finnigan MSQ single quad Mass Spectrometer (APCI)

HPLC column: Phenomenex Gemini 5 μ C18 110 A, 3.0×150 mm

HPLC gradient: 1.5 mL/min, from 5% acetonitrile in water to 90% acetonitrile in water in 9.86 minutes, from 90% acetonitrile in water to 95% acetonitrile in water in 0.1 minutes, hold at 95% for 1.19 minutes. Both acetonitrile and water have 0.04% NH₄OH

Preparative HPLC-MS

Platform: Shimadzu HPLC equipped with a Gilson 215 auto-sampler/fraction collector, UV detector and a PE Sciex API150EX mass detector;

HPLC column: BHK ODS-O/B, 5 μ, 30×75 mm

HPLC gradient: 35 mL/minute, 10% acetonitrile in water to 100% acetonitrile in 7 minutes, maintaining 100% acetonitrile for 3 minutes, with 0.025% TFA.

Analytical HPLC-MS Method 6

Platform: Agilent 1100 series: equipped with an auto-sampler, an UV detector (220 nM and 254 nM), a MS detector (electrospray);

HPLC column: XTerra MS, C₁₈, 5 μ, 3.0×250 mm column;

HPLC gradient: 1.0 mL/minute, from 10% acetonitrile in water to 90% acetonitrile in water in 46 minutes, jump to 99% acetonitrile and maintain 99% acetonitrile for 8.04 minutes. Both acetonitrile and water have 0.1% NH₄OH.

Chiral HPLC

Platform: Dionex P680A and P680P pumps, Dionex PAD 100 photodiode array detector, Jasco CD 2095 plus chiral detector, Gilson 215 liquid handler. Analytical Columns are 0.46×25 cm, 5 μm; preparative columns are 2×25 cm, 5 μm.

### Example 1

### [3-(4-CHLORO-PHENOXY)-2,3-DIHYDRO-BENZOFURAN-2-YLMETHYL]-METHYL-AMINE

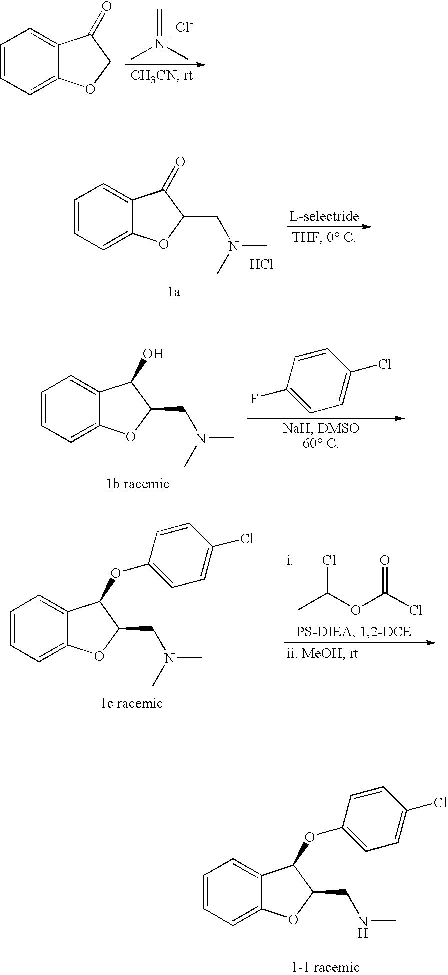 Step 1A:

To a solution of 3-coumaranone (1.00 g, 7.5 mmol) in dry acetonitrile (10 mL) was added N,N-dimethylmethyleneammonium chloride (0.76 g, 7.5 mmol). The resultant solution was stirred vigorously for 16 h. The precipitate was filtered under reduced pressure and washed with diethyl ether to obtain 1a as an off-white solid (1.25 g, 74%) that was used without further purification.

Step 1B:

To a stirred suspension of ketone 1a (0.70 g, 3.1 mmol) in dry THF (15 mL) at 0° C. was added L-selectride (1.0 M in THF, 7.8 mL, 7.8 mmol). The reaction mixture was allowed to warm gradually to room temperature. After stirring for 1 h, 10% aqueous sodium hydroxide (3 mL) was added. The reaction mixture was diluted with water (20 mL) and extracted with diethyl ether (3×20 mL). The organic extracts were dried over magnesium sulfate, filtered and concentrated in vacuo. The residue was purified by flash chromatography eluting with 0 to 15% methanol in dichloromethane to afford 1b as a pale yellow solid (0.46 g, 56%).

¹H NMR (300 MHz, CDCl₃) δ 7.43 (m, 1H), 7.23 (m, 1H), 6.94 (m, 1H), 6.84 (d, J=8.4 Hz, 1H), 5.41 (d, J=6.9 Hz, 1H), 4.61 (m, 1H), 3.04 (dd, J=13.5, 5.7 Hz, 1H), 2.96 (dd, J=13.5, 5.1 Hz, 1H), 2.35 (s, 6H). APCI MS m/e: 193.9 ([M+H]⁺).

Step 1C:

A suspension of sodium hydride (60% in mineral oil, 83 mg, 2.1 mmol) in DMSO (2 mL) under N₂ was heated at 60° C. for 15 min. A solution of alcohol 1b (200 mg, 1.1 mmol) in DMSO (2 mL) was added to the reaction mixture and heating was continued at 60° C. for a further 15 min. 1-Chloro-4-fluorobenzene (220 μL, 2.1 mmol) was added to the reaction mixture and heating was continued at 60° C. for a further 2h. The mixture was cooled to room temperature, diluted with saturated brine (30 mL), extracted with ethyl acetate (3×30 mL), dried over magnesium sulfate, filtered and concentrated in vacuo. The crude reaction mixture was purified by flash chromatography eluting with 0 to 50% ethyl acetate in hexanes to afford 1c as a yellow oil (160 mg, 51%).

¹H NMR (500 MHz, CDCl₃) δ 7.35 (m, 4H), 6.98 (m, 3H), 6.89 (t, J=7.5 Hz, 1H), 5.72 (d, J=6.0 Hz, 1H), 4.81 (ddd, J=8.5, 6.0, 4.0 Hz, 1H), 3.01 (dd, J=13.5, 8.5 Hz, 1H), 2.82 (dd, J=13.5, 4.0 Hz, 1H), 2.40 (s, 6H). APCI MS m/e: 303.8 ([M+H]⁺).

Step 1D:

To a solution of amine 1c (65 mg, 0.21 mmol) in 1,2-dichloroethane (3 mL) was added diisopropylamine polymer bound (PS-DIEA, 3.66 mmol/g, 460 mg, 1.68 mmol), followed by 1-chloroethylchloroformate (230 μL, 2.14 mmol). The reaction mixture was heated at 40° C. for 2.5 h then cooled to room temperature. PS-DIEA was removed by filtration under reduced pressure, and washed with dichloromethane (10 mL). The filtrate was concentrated in vacuo and the residue was dissolved in methanol (5 mL) and stirred at room temperature for 0.5 h. The reaction mixture was concentrated in vacuo and purified by flash chromatography eluting with 0 to 6% methanol in dichloromethane to afford 1-1 as a white solid (21 mg, 34%).

¹H NMR (300 MHz, CDCl₃) δ 7.27 (m, 4H), 6.91 (m, 4H), 5.72 (d, J=6.6 Hz, 1H), 4.85 (ddd, J=8.4, 6.6, 3.9 Hz, 1H), 3.17 (dd, J=12.9, 8.4 Hz, 1H), 3.03 (dd, J=12.9, 3.9 Hz, 1H), 2.52 (s, 3H). APCI MS m/e: 289.8 ([M+H]⁺).

### Example 2

### [4-(4-CHLORO-PHENOXY)-CHROMAN-3-YLMETHYL]-METHYL-AMINE

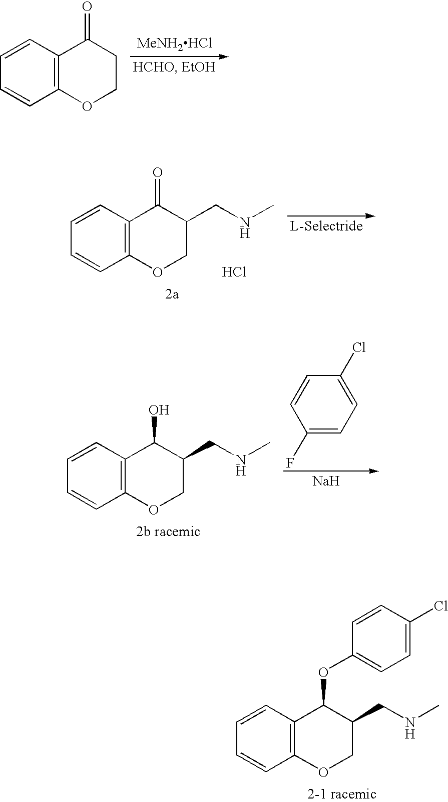 Step 2A:

A suspension of 4-chromanone (3.23 g, 21.8 mmol), paraformaldehyde (1.97 g, 65.4 mmol) and methylamine hydrochloride (7.36 g, 109 mmol) in ethanol (60 mL) was treated with 2.0 M hydrochloric acid in ether (0.4 mL). The mixture was heated to reflux. After 3 hours, one more equivalent each of amine and aldehyde was added. The reaction was removed from heat after 6 hours of stirring and allowed to cool to room temperature. A portion of the crude reaction mixture (approximately one third) was diluted with water and washed with ether. The aqueous layer was then made basic with potassium carbonate and extracted with ether. The ether extract was dried over magnesium sulfate and evaporated to give 2a as an orange oil (1.32 g).

APCI MS m/e: 191.9 ([M+H]⁺)

Step 2B:

A solution of 2a (1.32 g, 6.9 mmol) in THF (35 mL) was cooled to 0° C. To this mixture, L-Selectride (1.0 M in THF, 17.2 mL) was added slowly. The mixture was allowed to slowly warm up to room temperature. The reaction was quenched with a 10% sodium hydroxide solution (5 mL). After removal of the volatiles, the residue was partitioned between water and ether (20 mL each). The ether layer was washed with 0.5 N hydrochloric acid (20 mL). This aqueous layer was made basic with solid sodium hydroxide to pH 12 and extracted with 5:1 ethyl acetate/isopropanol (3×20 mL). The combined organic layers were dried and over magnesium sulfate and evaporated to give 2b as a light yellow oil (200 mg). APCI MS m/e: 193.9 ([M+H]⁺)

Step 2C:

Sodium hydride (26 mg, 0.65 mmol, 40% dispersion in oil) was added to dry DMSO (1.0 mL) and the suspension heated in a 60° C. bath. After 20 minutes 2b (50 mg, 0.26 mmol) was added as a solution in DMSO (0.5 mL). After 15 minutes at this temperature, 4-chlorofluorobenzene (41 μL, 0.39 mmol) was added and the bath temperature raised to 90° C. The reaction mixture was heated overnight. After cooling, the mixture was diluted slightly with methanol and purified on a prep HPLC collecting by mass signal to give 10 mg of 2-1 as the trifluoroacetate salt.

¹H NMR (300 MHz, CDCl₃) δ 9.79 (br s, 1H), 9.63 (br s, 1H), 7.22-7.29 (m, 3H), 6.96-7.00 (m, 3H), 6.82-6.86 (m, 2H), 5.44 (d, J=4.0 Hz, 1H), 4.41 (dd, J=11.5, 8.0 Hz, 1H), 4.32 (dd, J=11.5, 2.5 Hz, 1H), 3.25-3.33 (m, 1H), 3.04-3.12 (m, 1H), 2.72-2.76 (m, 1H), 2.65 (s, 3H). APCI MS m/e: 303.8 ([M+H]⁺).

Following the same procedure, [4-phenoxy-chroman-3-ylmethyl]-methyl-amine 2-2 ([M+H]⁺=269.9) and [4-(3-chloro-phenoxy)-chroman-3-ylmethyl]-methyl-amine 2-3 ([M+H]⁺=303.8) were prepared.

### Example 3

### [4-(3,4-DICHLORO-PHENOXY)-CHROMAN-3-YLMETHYL]-METHYL-AMINE

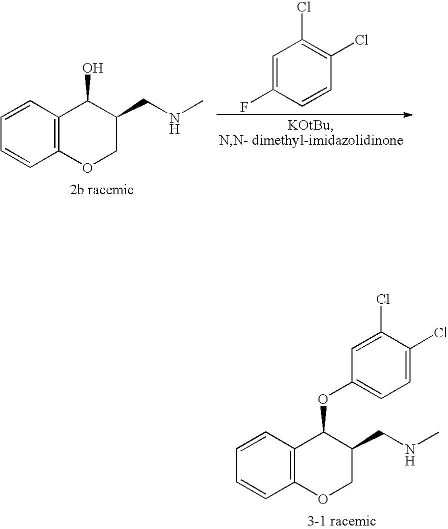 Step 3A:

The aminoalcohol 2b (64 mg, 0.33 mmol) was dissolved in 1,3-dimethylimidazolidinone (0.5 mL) and treated with potassium tert-butoxide (77 mg, 0.69 mmol). 3,4-Dichlorofluorobenzene (163 mg, 0.99 mmol) was added next and the mixture was heated in a 110° C. bath overnight. After cooling, the reaction mixture was diluted with some methanol and purified on a prep HPLC collecting by mass signal to give 3-1 as the trifluoroacetate salt (7 mg).

¹H NMR (300 MHz, CDCl₃) δ 9.61-9.80 (br s, 2H), 7.35 (d, J=8.8 Hz, 1H), 7.26 (s, 1H), 7.19-7.23 (m, 1H), 7.14 (d, J=2.8 Hz, 1H), 6.96 (dd, J=7.8, 1.5 Hz, 1H), 6.90 (dd, J=8.9, 2.8 Hz, 1H), 6.79-6.83 (m, 1H), 5.46 (d, J=3.8 Hz, 1H), 4.37 (dd, J=11.5, 8.6 Hz, 1H), 4.30 (dd, J=11.7, 2.9 Hz, 1H), 3.23-3.32 (m, 1H), 3.04-3.12 (m, 1H), 2.73-2.78 (m, 1H), 2.67 (s, 3H). APCI MS m/e: 337.7 ([M+H]⁺)

### Example 4

### [4-(2-CHLORO-4-METHYL-PHENOXY)-CHROMAN-3-YLMETHYL]-METHYL-AMINE

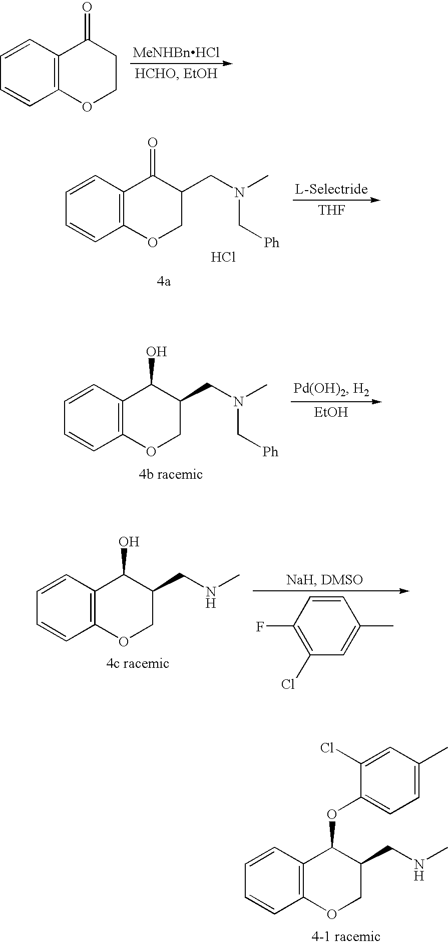 Step 4A:

To a solution of N-methylbenzylamine (16.3 g, 132 mmol) in ethanol (250 mL), stirring at 0° C., was slowly added HCl (2N in ether, 66 mL, 132 mmol). To the resultant white slurry was added 4-chromanone (10 g, 67.5 mmol), followed by paraformaldehyde (6 g, 200 mmol). The reaction mixture was heated to 90° C. and refluxed 18 hours. The reaction mixture was then concentrated in vacuo. The excess N-methylbenzylamine was removed via repeated crystallization from hot ethyl acetate. The resulting mother liquor was concentrated in vacuo and dissolved in minimal ethanol (20 mL). This solution was slowly added to vigorously stirring ether (800 mL), to afford 4a as a gray-colored solid (10.8 g, 48% yield) which contained a slight amount of N-methylbenzylamine (<10%).

¹H NMR (300 MHz, CDCl3) δ 7.80-7.90 (m, 1H), 7.35-7.68 (m, 6H), 6.05-7.05 (m, 2H), 5.05-5.20 (m, 1H), 4.05-4.30 (m, 2H), 3.40-3.75 (m, 2H), 2.79 (d, J=4.5 Hz, 1H), 2.68 (d, J=4.5 Hz, 1H), 1.63 (s, 3H). APCI MS m/e: 282.0 ([M+H]⁺)

Step 4B:

The crystalline hydrochloride salt 4a (3.0 g, 10.7 mmol) was suspended in THF (40 mL) and cooled to 0° C. L-Selectride (1.0 M in THF, 26.7 mL) was added slowly to the suspension. After half an hour, the reaction was quenched with a 10% sodium hydroxide solution (5 mL) and removed from the cold bath. After evaporating the volatiles, the residue was partitioned between water and ether (20 mL each). The ether layer was washed with a 0.5 N solution of hydrochloric acid (2×15 mL). The aqueous layer was made basic to pH 12 with solid sodium hydroxide. The basic aqueous layer was then extracted with ether (2×15 mL). The combined organic layers were dried over magnesium sulfate and evaporated to give 2.5g of the racemic, predominantly cis alcohol 4b, as a viscous clear oil.

¹H NMR (300 MHz, CDCl₃) δ 7.51 (d, J=6.9 Hz, 1H), 7.12-7.33 (m, 6H), 6.94 (dt, J=7.5, 1.2 Hz, 1H), 6.74 (dd, J=8.1, 1.2 Hz, 1H), 4.99 (d, J=4.2 Hz, 1H), 4.16 (dd, J=11.4, 2.7 Hz, 1H), 4.02 (dd, J=11.4, 6.3 Hz, 1H), 3.66 (d, J=12.9 Hz, 1H), 3.34 (d, J=12.9 Hz, 1H), 2.90 (dd, J=13.2, 12.0 Hz, 1H), 2.45-2.53 (m, 2H), 2.24 (s, 3H). APCI MS m/e: 283.9 ([M+H]⁺)

Step 4C:

To a solution of 4b (1.33 g, 4.73 mmol) in ethanol (100 mL) was added palladium hydroxide (20% on carbon, 658 mg, 0.47 mmol). The suspension was stirred under atmospheric hydrogen for half an hour. The mixture was filtered through Celite, using ethanol to wash. On evaporation, 4c was obtained as a viscous, clear oil (800 mg), which was used without purification.

¹H NMR (300 MHz, CDCl₃) δ 7.44 (dd, J=7.5, 1.2 Hz, 1H), 7.17 (ddd, J=7.8, 7.8, 1.7 Hz, 1H), 6.93 (ddd, J=7.5, 7.5, 1.2 Hz, 1H), 6.78 (dd, J=8.1, 0.9 Hz, 1H), 4.91 (d, J=4.2 Hz, 1H), 4.10-4.13 (m, 2H), 2.96 (dd, J=12.3, 9.3 Hz, 1H), 2.85 (dd, J=12.3, 4.2 Hz, 1H), 2.41 (s, 3H), 2.26-2.35 (m, 1H). APCI MS m/e: 193.9 ([M+H]⁺)

Step 4D:

Sodium hydride (28 mg, 0.7 mmol) was added to dry DMSO (0.75 mL) and the resulting suspension was stirred at 60° C. for half an hour. The aminoalcohol 4c (100 mg, 0.35 mmol) was added as a solution in DMSO (0.75 mL) and the stirring continued at 60° C. for another 15 minutes. Lastly, 3-chloro-4-fluorotoluene (63 μL, 0.52 mmol) was added in and the mixture heated at 90° C. overnight. After dilution with 0.5 mL of methanol, the mixture was purified on an HPLC/MS collecting by MS signal to afford [4-(2-chloro-4-methyl-phenoxy)-chroman-3-ylmethyl]-methyl-amine 4-1 as the trifluoroacetate salt (10 mg).

¹H NMR (300 MHz, CDCl₃) δ 10.10 (br s, 1H), 9.17 (br s, 1H), 7.18-7.24 (m, 3H), 6.73-7.01 (m, 4H), 5.45 (d, J=3.6 Hz, 1H), 4.48 (dd, J=11.4, 8.1 Hz, 1H), 4.37 (dd, J=11.4, 3.0 Hz, 1H), 3.23-3.67 (m, 3H), 2.74 (s, 3h), 2.29 (s, 3H). APCI MS m/e: 317.8 ([M+H]⁺)

The following compounds were made according to this procedure.

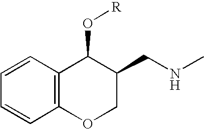

| No. | R | Stereochem | MW | MH⁺ | t_{R} (method 2) |
|---|---|---|---|---|---|
| 4-1 | 2-Cl-4-CH₃-phenyl | R,R and S,S | 317.8 | 317.8 | 5.36 |
| 4-2 | 4-Br-2-CH₃-phenyl | R,R and S,S | 362.3 | 364.0 | 5.90 |
| 4-3 | 4-Cl-2-CH₃-phenyl | R,R and S,S | 317.8 | 317.8 | 5.64 |
| 4-4 | 1-naphthyl | R,R and S,S | 319.4 | 319.9 | 5.17 |
| 4-5 | 2-CH₃-4-NO₂-phenyl | R,R and S,S | 328.4 | 329.0 | 5.00 |
| 4-6 | 2-Br-phenyl | R,R and S,S | 348.2 | 349.9 | 4.90 |
| 4-7 | 2-Cl-phenyl | R,R and S,S | 303.8 | 304.0 | 4.92 |
| 4-8 | 3-Br-2-CH₃-phenyl | R,R and S,S | 362.3 | 362.0 | 5.68 |
| 4-9 | 3-F-2-CH₃-phenyl | R,R and S,S | 301.4 | 302.0 | 5.19 |
| 4-10 | 2-F-phenyl | R,R and S,S | 287.3 | 288.0 | 4.58 |
| 4-11 | 2-vinyl-phenyl | R,R and S,S | 295.4 | 296.1 | 5.18 |
| 4-12 | 2-Cl-phenyl | R,R | 303.8 | 304.0 | 4.95 |
| 4-13 | 2-Cl-phenyl | S,S | 303.8 | 304.0 | 4.93 |

During purification of 4-9 by prep. HPLC, a minor amount of the trans racemic mixture was isolated.

| | |
|---|---|
| | racemic |
| 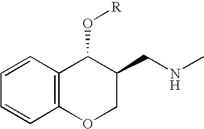 | |

| No. | R | MW | MH⁺ | t_{R} (method 2) |
|---|---|---|---|---|
| 4-14 | 3-F-2-CH₃-phenyl | 301.4 | 302.0 | 5.24 |

### Example 5

### 2-(3-METHYLAMINOMETHYL-CHROMAN-4-YLOXY)-BENZONITRILE

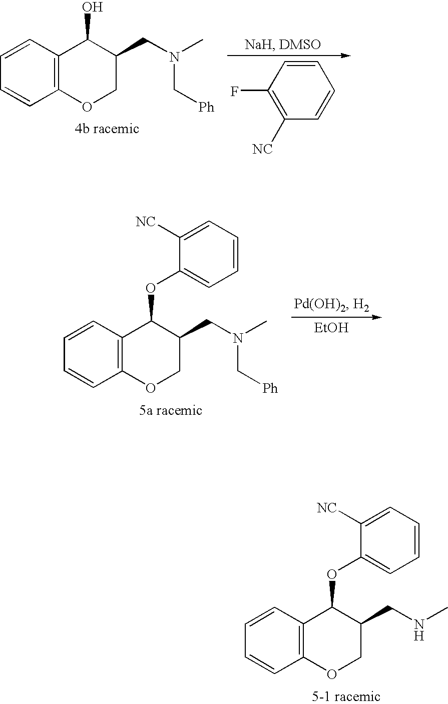 Step 5A:

Sodium hydride (127 mg, 3.18 mmol) was added to dry DMSO (2 mL) and heated at 60° C. for half an hour. The aminoalcohol 4b (200 mg, 0.71 mmol) was added to this mixture and heating continued for another 15 minutes. Finally, 2-fluorobenzonitrile (385 mg, 3.18 mmol) was added and the mixture heated at 90° C. overnight. The reaction mixture was diluted with methanol and purified in batches on a prep HPLC/MS to yield 5a as a tan oil (150 mg). APCI MS m/e: 384.8 ([M+H]⁺)

Step 5B:

The benzylamine 5a (153 mg, 0.4 mmol) was dissolved in ethanol (3 mL) and treated with palladium hydroxide (56 mg, 0.04 mmol). Hydrogen gas was introduced via balloon. After half an hour, hydrogen was removed and the reaction mix was filtered through a plug of Celite, using ethanol to wash the solids. Excess solvent was removed in vacuo and the residue purified via HPLC chromatography to give 2-(3-methylaminomethyl-chroman-4-yloxy)-benzonitrile 5-1 as the trifluoroacetate salt.

¹H NMR (300 MHz, CDCl₃) δ 9.69 (br s, 1H), 9.01 (br s, 1H), 6.75-7.57 (m, 8H), 5.71 (s, 1H), 4.49-4.55 (m, 1H), 4.37 (d, J=11.1 Hz, 1H), 3.46-3.50 (m, 1H), 3.24-3.29 (m, 1H), 2.87-2.91 (m, 1H), 2.77 (s, 3H). APCI MS m/e: 294.8 ([M+H]⁺)

### Example 6

### METHYL-[4-(NAPHTHALEN-1-YLOXY)-CHROMAN-3-YLMETHYL]-AMINE

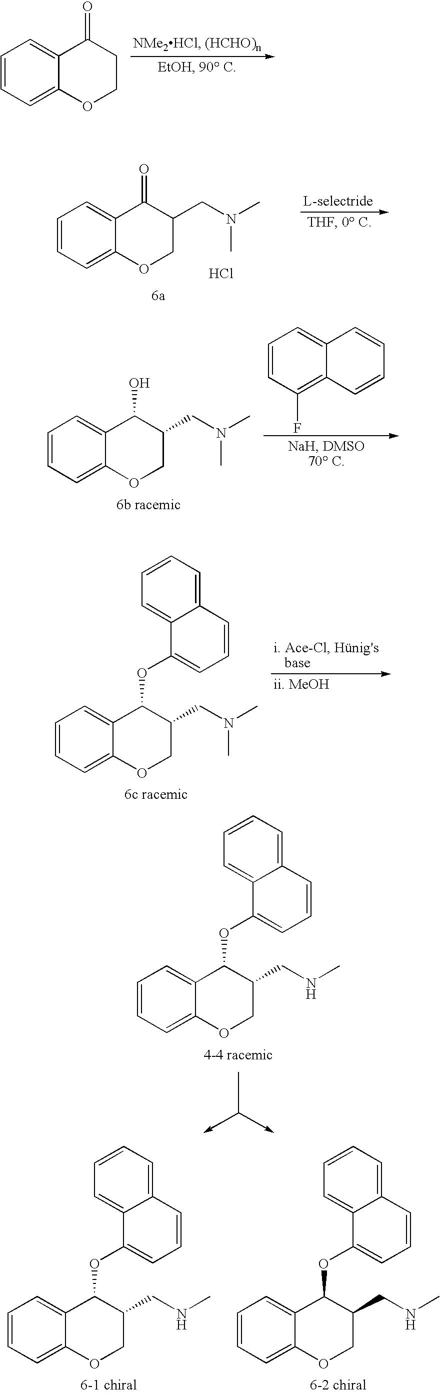 Step 6A:

To a suspension of dimethylamine hydrochloride (33.0 g, 405 mmol) in ethanol (400 mL) was added HCl (2M in diethyl ether, 20 mL) followed by paraformaldehyde (20 g, 675 mmol) and 4-chromanone (20 g, 135 mmol). The mixture was heated at 90° C. for 16 h then cooled to room temperature. The mixture was concentrated to half volume and the precipitate was filtered under reduced pressure and washed with diethyl ether to obtain 6a as white crystals (23.1 g, 71%). This material was used without further purification.

¹H NMR (300 MHz, CD₃OD) δ 7.89 (m, 1H), 7.59 (ddd, J=8.7, 7.5, 2.1 Hz, 1H), 7.07 (m, 2H), 4.68 (dd, J=11.4, 5.4 Hz, 1H), 4.29 (t, J=11.4 Hz, 1H), 3.61 (m, 2H), 3.31 (app. quint, J=1.8 Hz, 1H), 3.20 (m, 1H), 3.00 (s, 6H). APCI MS m/e: 206.0 ([M+H]⁺).

Step 6B:

To a stirred suspension of ketone 6a (18.6 g, 77.2 mmol) in dry THF (400 mL) at 0° C. was added L-selectride (1.0 M in THF, 85.0 mL, 84.9 mmol). The reaction mixture was allowed to warm gradually to room temperature. After stirring for 16 h the reaction mixture was concentrated in vacuo and partitioned between 10% aqueous sodium hydroxide (200 mL) and ethyl acetate (300 mL). The aqueous layer was extracted with ethyl acetate (300 mL) and the combined organic extracts were washed with brine (300 mL), dried over magnesium sulfate, filtered and concentrated in vacuo. Purification by flash chromatography eluting with 0 to 8% methanol in dichloromethane with 0.1% triethylamine afforded 6b as a pale yellow oil (7.95 g, 50%).

¹H NMR (300 MHz, CDCl₃) δ 7.52 (m, 1H), 7.18 (m, 1H), 6.96 (td, J=7.5, 1.2 Hz, 1H) 6.77 (dd, J=8.4, 1.5 Hz, 1H), 4.76 (d, J=4.8 Hz, 1H), 4.18 (dd, J=11.7, 2.7 Hz, 1H), 4.04 (dd, J=11.7, 5.4 Hz, 1H), 2.82 (t, J=13.0 Hz, 1H), 2.41 (m, 2H), 2.27 (s, 6H). APCI MS m/e: 208.0 ([M+H]⁺).

Step 6C:

To a suspension of sodium hydride (60% in mineral oil, 40 mg, 1.0 mmol) in DMSO (1.0 mL) was added a solution of alcohol 6b (200 mg, 1.0 mmol) in DMSO (0.5 mL) followed by 1-fluoronaphthalene (425 mg, 2.9 mmol). The reaction was heated at 70° C. for 16 h. A further portion of sodium hydride (60% in mineral oil, 20 mg, 0.5 mmol) was added and heating was continued for 4 h. The mixture was cooled to room temperature, diluted with brine (15 mL) and distilled water (15 mL) and extracted with ethyl acetate (3×30 mL), dried over magnesium sulfate, filtered and concentrated in vacuo. The crude reaction mixture was purified by flash chromatography eluting with 0 to 40% ethyl acetate in hexanes to afford 6c as a yellow oil (73 mg, 23%). ¹H NMR (300 MHz, CDCl₃) δ 8.09 (m, 1H), 7.79 (d, J=7.5 Hz, 1H), 7.40 (m, 4H), 7.25 (m, 1H), 7.19 (ddd, J=8.4, 7.2, 1.8 Hz, 1H), 7.06 (dd, J=7.8, 1.8 Hz, 1H), 7.19 (dd, J=8.1, 1.2 Hz, 1H), 6.72 (td, J=7.2, 0.9 Hz, 1H), 5.64 (d, J=2.7 Hz, 1H), 4.51 (t, J=10.8 Hz, 1H), 4.35 (m, 1H), 2.57 (m, 2H), 2.41 (dd, J=14.7, 10.2 Hz, 1H), 2.20 (s, 6H). APCI MS m/e: 334.1 ([M+H]⁺).

Step 6D:

To a solution of amine 6c (73 mg, 0.22 mmol) in 1,2-dichloroethane (5 mL) was added diisopropylethylamine (50 μL, 15.2 mmol), followed by 1-chloroethylchloroformate (73 μL, 0.44 mmol). The reaction mixture was heated at 40° C. for 5 h then cooled to room temperature. Methanol (10 mL) was added to the residue and the solution was made basic (pH 8.0 with universal indicator paper) by the dropwise addition of 2M ammonia in methanol. This solution was stirred at room temperature for 4 d then concentrated in vacuo and purified by flash chromatography to afford methyl-[4-(naphthalen-1-yloxy)-chroman-3-ylmethyl]-amine 4-4 as a pale yellow oil (25 mg, 35%). ¹H NMR (300 MHz, CDCl₃) δ 8.10 (m, 1H), 7.80 (m, 1H), 7.17-7.51 (m, 7H), 6.89 (m, 1H), 6.77 (m, 1H), 5.74 (d, J=3.3 Hz, 1H), 4.54 (dd, J=10.5, 9.6 Hz, 1H), 4.38 (ddd, J=10.5, 3.0, 0.9 Hz, 1H), 2.93 (dd, J=12.0, 7.5 Hz, 1H), 2.80 (dd, J=12.0, 7.5 Hz, 1H), 2.61 (m, 1H), 2.39 (s, 3H). APCI MS m/e: 320.1 ([M+H]⁺).

Step 6E:

The enantiomerically pure compounds, 6-1 and 6-2 (8 mg each, 64% recovery), were obtained from racemic 4-4 (25 mg) by chiral preparative HPLC using a Chiralpak AD-H (20×250 cm) column eluting with 17:3 hexanes/ethanol with 0.1% diethylamine at a flow rate of 15 mL/min.

Starting with alcohol 6b the following compounds were made according to this procedure:

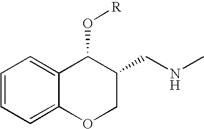

| No. | R | Stereochem. | MW | MH⁺ | t_{R} (method) |
|---|---|---|---|---|---|
| 6-3 | 2-Me-3-F-phenyl | S,S | 301.4 | 302.0 | 9.60 (5) |
| 6-4 | 2-Me-3-F-phenyl | R,R | 301.4 | 302.0 | 9.10 (5) |
| 6-5 | 2-CF₃-phenyl | S,S and R,R | 337.3 | 338.0 | 6.87 (2) |
| 6-6 | 2-CF₃-phenyl | R,R | 337.3 | 338.1 | 9.29 (5) |
| 6-7 | 2-CF₃-phenyl | S,S | 337.3 | 338.0 | 9.05 (5) |

### Example 7

### METHYL-(4-O-TOLYLOXY-CHROMAN-3-YLMETHYL)-AMINE

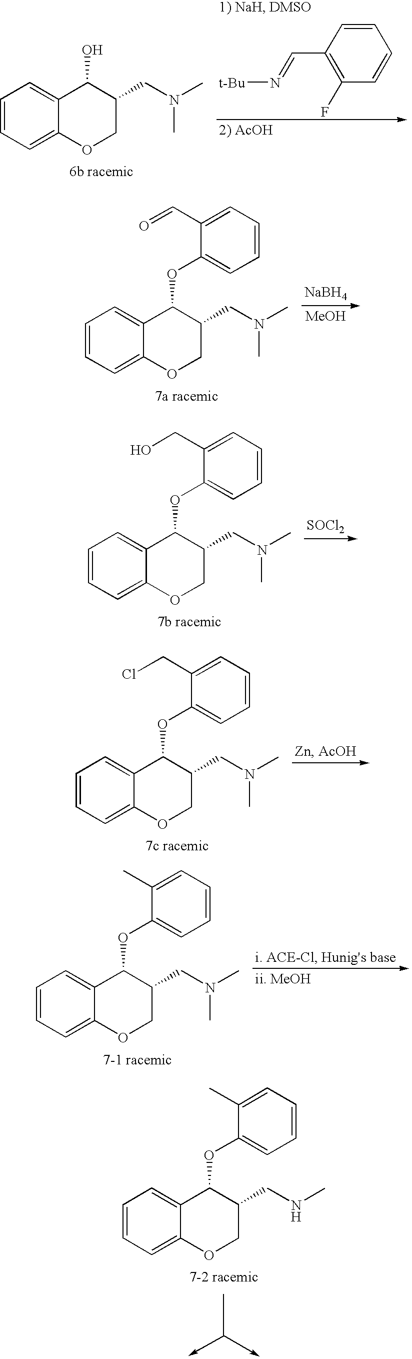 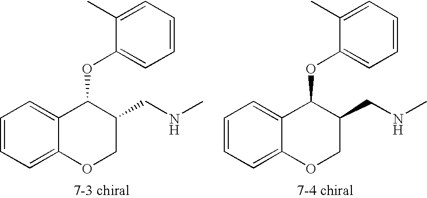 Step 7A:

To a suspension of sodium hydride (60% in mineral oil, 1.82 g, 45.5 mmol) in DMSO (100 mL) under N₂ was added a solution of alcohol 6b (8.60 g, 41.4 mmol) in DMSO (20 mL). The mixture was stirred at room temperature for 15 min. Potassium benzoate (6.62 g, 41.4 mmol) was added in one portion and the mixture was stirred for a further 15 min. 1-Fluoro-2-(t-butylimino)benzene (11.2 g, 62.1 mmol, made from the reaction of 2-fluorobenzaldehyde (34.2 mL, 0.32 mol) and t-butylamine (34.8 mL, 0.34 mol) in DCM (400 mL) with 4 Å powdered activated molecular sieves (160 g), stirring for 16 h at room temperature, filtering through celite, concentrating in vacuo and vacuum distilling to afford 36.1 g of a colorless oil) was added and the reaction was heated at 70° C. for 90 min. The mixture was cooled to 0° C. in a 0° C. bath and acetic acid (7 mL) in distilled water (70 mL) was added slowly to the reaction mixture. After stirring at room temperature for 3 h, the solution was partitioned between ethyl acetate (400 mL) and distilled water (400 mL). The aqueous phase was extracted with ethyl acetate (400 mL); the combined organics were washed with saturated aqueous sodium bicarbonate (2×400 mL), dried over magnesium sulfate, filtered and concentrated in vacuo. Purification by flash chromatography eluting with 0 to 40% ethyl acetate in hexanes gave 7a as a dark yellow oil (7.20 g, 56%). ¹H NMR (300 MHz, CDCl₃) δ 10.07 (s, 1H), 7.79 (dd, J=7.8, 2.1 Hz, 1H), 7.61 (ddd, J=8.4, 7.2, 1.8 Hz, 1H), 7.46 (m, 1H), 7.22 (ddd, J=8.7, 7.2, 1.8 Hz, 1H), 7.13 (m, 1H), 6.90 (m, 1H), 6.84 (dd, J=7.5, 1.5 Hz, 1H), 6.73 (td, J=7.5, 1.2 Hz, 1H), 5.44 (d, J=2.7 Hz, 1H), 4.29 (m, 2H), 2.59 (m, 2H), 2.31 (m, 1H), 2.26 (s, 6H). APCI MS m/e: 312.0 ([M+H]⁺).

Step 7B:

To a stirred solution of the aldehyde 7a (7.20 g, 23.2 mmol) in methanol (200 mL) at 0° C. (internal temperature) was added sodium borohydride (0.97 g, 25.5 mmol) portionwise. The mixture was stirred for 30 min then concentrated in vacuo and partitioned between distilled water (200 mL) and ethyl acetate (200 mL). The aqueous phase was extracted with ethyl acetate (2×200 mL), the combined organics were dried over magnesium sulfate, filtered and concentrated in vacuo to afford 7b as a dark yellow oil (7.00 g, 97%). ¹H NMR (300 MHz, CDCl₃) δ 7.27 (td, J=7.5, 1.5 Hz, 1H), 7.22 (dt, J=6.6, 1.8 Hz, 1H), 7.17 (dd, J=7.5 and 2.1 Hz, 1H), 7.02 (td, J=7.5 and 1.2 Hz, 1H), 6.90 (d, J=7.8 Hz, 1H), 6.77 (m, 3H), 5.32 (d, J=3.0 Hz, 1H), 4.68 (d, J=12.3 Hz, 1H), 4.35 (dd, J=10.8, 7.2 Hz, 1H), 4.23 (ddd, J=10.8, 3.6, 0.9 Hz, 1H), 2.91 (dd, J=12.3, 9.6 Hz, 1H), 2.50 (m, 1H), 2.30 (s, 6H), 2.24 (m, 2H). APCI MS m/e: 314.0 ([M+H]⁺).

Step 7C:

To a stirred solution of the alcohol 7b (7.00 g, 22.4 mmol) in dichloromethane (200 mL) at 0° C. (internal temperature) was added thionyl chloride (1.86 mL, 25.5 mmol) in dichloromethane (25 mL) dropwise. The mixture was stirred for 30 min then concentrated in vacuo to afford 7c as a pale brown solid. This material was used in the next step without further purification. APCI MS m/e: 332.0 ([M+H]⁺).

Step 7D:

To a stirred solution of the benzyl chloride 7c (22.4 mmol) in acetic acid (60 mL) and distilled water (3.6 mL) was added zinc powder (9.0 g, 139.2 mmol) in one portion. The mixture was stirred vigorously for 3 h then filtered through Celite and washed with dichloromethane (2×200 mL) and methanol (250 mL). The filtrate was concentrated in vacuo and partitioned between saturated aqueous ammonium chloride (120 mL) and dichloromethane (250 mL). The aqueous phase was extracted with dichloromethane (2×100 mL), the combined organics were washed with 1:2 distilled water/saturated aqueous sodium bicarbonate (2×200 mL), dried over magnesium sulfate, filtered and concentrated. Purification via flash chromatography (0-25% ethyl acetate in hexanes) afforded dimethyl-(4-o-tolyloxy-chroman-3-ylmethyl)-amine 7-1 as a white solid (5.90 g, 86%). ¹H NMR (300 MHz, CDCl₃) δ 7.18 (m, 3H), 6.80 (m, 1H), 6.85 (m, 3H), 6.71 (m, 1H), 5.27 (d, J=3.6 Hz, 1H), 4.39 (t, J=10.5 Hz, 1H), 4.28 (dd, J=10.5, 3.0 Hz, 1H), 2.56 (m, 1H), 2.43 (m, 2H), 2.24 (s, 6H), 1.94 (s, 3H). APCI MS m/e: 298.0 ([M+H]⁺).

Step 7E:

To a solution of 7-1 (3.20 g, 10.8 mmol) in 1,2-dichloroethane (75 mL) was added diisopropylethylamine (3.6 mL, 21.6 mmol), followed by 1-chloroethylchloroformate (5.8 mL, 5.4 mmol). The reaction mixture was heated at 40° C. for 1 h then cooled to room temperature. The reaction mixture was partitioned between dichloromethane (100 mL) and saturated aqueous sodium bicarbonate (100 mL). The aqueous phase was extracted with dichloromethane (100 mL) and the combined organics were dried over magnesium sulfate, filtered and concentrated in vacuo. The residue was dissolved in methanol (60 mL) then the solution was made basic (pH 8.0 with universal indicator paper) by the dropwise addition of 2M ammonia in methanol. This solution was stirred at room temperature for 2 h then concentrated in vacuo and partitioned between dichloromethane (100 mL) and distilled water (100 mL). The organic layer was dried over magnesium sulfate, filtered and concentrated in vacuo, redissolved in methanol (60 mL) and stirred for a further 3h. The solution was again concentrated and the residue was purified by flash chromatography eluting with 0 to 6% methanol in dichloromethane to afford methyl-(4-o-tolyloxy-chroman-3-ylmethyl)-amine 7-2 as a pale orange solid (2.33 g, 76%).

¹H NMR (300 MHz, CDCl₃) δ 7.09-7.22 (m, 4H), 6.84-6.96 (m, 3H), 6.74 (dt, J=7.5, 1.2 Hz, 1H), 5.37 (d, J=3.3 Hz, 1H), 4.40 (dd, J=10.5, 10.5 Hz, 1H), 4.31 (dd, J=10.8, 3.9 Hz, 1H), 2.91 (dd, J=12.0, 7.2 Hz, 1H), 2.75 (dd, J=12.0, 6.9 Hz, 1H), 2.38-2.50 (m, 1H), 2.44 (s, 3H), 1.99 (s, 3H). APCI MS m/e: 283.9 ([M+H]⁺).

Step 7F:

The enantiomerically pure compounds, 7-3 and 7-4 (338 mg each, 67% recovery), were obtained from racemic 7-2 (1.0 g) by chiral preparative HPLC using a Chiralcel OD-H column eluting with 9:1 hexanes/ethanol with 0.1% diethylamine at a flow rate of 15 mL/min.

The following additional compound were made according to this procedure.

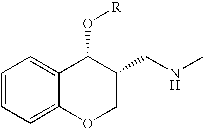

| No. | R | Stereochem. | MW | MH⁺ | t_{R} (method) |
|---|---|---|---|---|---|
| 7-5 | 4-F-2-Me-phenyl | S,S and R,R | 301.4 | 302.0 | 7.91 (5) |

Starting with alcohol 12b, the following compounds were made according to this procedure.

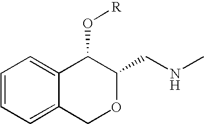

| No. | R | Stereochem. | MW | MH⁺ | t_{R} (method) |
|---|---|---|---|---|---|
| 7-6 | 4-F-2-Me-phenyl | S,S and R,R | 301.4 | 302.1 | 7.14 (5) |
| 7-7 | 6-F-2-Me-phenyl | S,S and R,R | 301.4 | 301.9 | 10.32 (5) |
| 7-8 | 6-F-2-Me-phenyl | S,S | 301.4 | 302.2 | 27.78 (6) |
| 7-9 | 6-F-2-Me-phenyl | R,R | 301.4 | 302.0 | 27.59 (6) |
| 7-10 | 5-F-2-Me-phenyl | S,S and R,R | 301.4 | 302.0 | 4.72 (2) |
| 7-11 | 3-F-4-Me-phenyl | S,S and R,R | 301.4 | 302.0 | 4.74 (2) |

### Example 8

### 2-(3-DIMETHYLAMINOMETHYL-CHROMAN-4-YLOXY)-BENZALDEHYDE

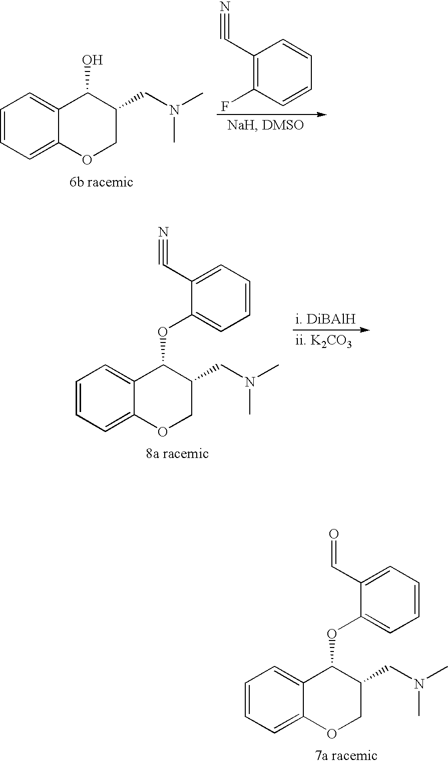 Step 8A:

To a suspension of sodium hydride (60% in mineral oil, 58 mg, 1.45 mmol) in DMSO (1.0 mL) was added a solution of alcohol 6b (300 mg, 1.45 mmol) in DMSO (0.5 mL). The mixture was cooled to 0° C. and 1-fluorobenzonitrile (530 mg, 4.35 mmol) was added. The reaction was allowed to warm to room temperature and stirred for 1 h. A further portion of sodium hydride (60% in mineral oil, 29 mg, 1.22 mmol) was added and stirring continued for 1 h. The mixture was then diluted with brine (15 mL) and distilled water (15 mL) and extracted with ethyl acetate (3×30 mL), dried over magnesium sulfate, filtered and concentrated in vacuo. The crude reaction mixture was purified by flash chromatography eluting with 0 to 60% ethyl acetate in hexanes to afford 8a as a pale yellow oil (362 mg, 81%). ¹H NMR (300 MHz, CDCl₃) δ 7.55 (m, 2H), 7.39 (d, J=8.4 Hz, 1H), 7.24 (ddd, J=8.4, 6.6, 1.2 Hz, 1H), 7.04 (m, 2H), 6.91 (dd, J=8.1, 0.9 Hz, 1H), 6.79 (td, J=7.5, 0.9 Hz, 1H), 5.50 (d, J=2.4 Hz, 1H), 4.44 (t, J=10.8 Hz, 1H), 4.25 (ddd, J=10.8, 3.9, 1.2 Hz, 1H), 2.56 (dd, J=11.4, 8.1 Hz, 1H), 2.46 (m, 1H), 2.29 (dd, J=11.4, 5.4 Hz, 1H), 2.18 (s, 6H). APCI MS m/e: 309.0 ([M+H]⁺).

Step 8B:

To a stirred solution of benzonitrile 8a (50 mg, 0.16 mmol) in dichloromethane (1.0 mL) under N₂ at −15° C. was added diisobutylaluminum hydride (DiBAL-H, 1.0 M in toluene, 0.16 mL, 0.16 mmol). The mixture was stirred at this temperature for 1 h and then a further portion of DiBAL-H was added (1.0 M in toluene, 0.16 mL, 0.16 mmol). After stirring for 1 h the reaction mixture was carefully added to 20% aqueous ammonium chloride (0.75 mL), 2 M hydrochloric acid (1.1 mL), distilled water (10 mL) and dichloromethane (5 mL). The resultant mixture was stirred vigorously for 2.5 h, the layers were separated and the aqueous phase was extracted with dichloromethane (2×10 mL). The combined organics were dried over magnesium sulfate, filtered and concentrated in vacuo to afford the hydrochloride salt as a white solid (30 mg, 60%). ¹H NMR (300 MHz, CDCl₃) δ 10.17 (s, 1H), 7.79 (dd, J=7.5, 1.8 Hz, 1H), 7.67 (ddd, J=8.4, 7.5, 2.1 Hz, 1H), 7.54 (d, J=8.7 Hz, 1H), 7.22 (m, 2H), 6.98 (dd, J=7.5, 1.5 Hz, 1H), 6.88 (dd, J=8.7, 1.2 Hz, 1H), 6.78 (dt, J=7.8, 0.9 Hz, 1H), 6.01 (d, J=3.9 Hz, 1H), 4.74 (dd, J=11.7, 8.1 Hz, 1H), 4.24 (dd, J=11.4, 2.7 Hz, 1H), 3.54 (dd, J=13.2, 4.2 Hz, 1H), 3.13 (dd, J=13.2, 6.0 Hz, 1H), 3.00 (m, 1H), 2.85 (s, 6H). APCI MS m/e: 312.0 ([M+H]⁺). The hydrochloride salt was partitioned between dichloromethane (10 mL) and sat. aqueous sodium carbonate (10 mL), the layers were separated and the dichloromethane layer was dried over magnesium sulfate, filtered and concentrated in vacuo to afford 2-(3-dimethylaminomethyl-chroman-4-yloxy)-benzaldehyde 7a as a colorless oil (identical ¹H NMR as reported previously for 7a).

### EXAMPLE 9

### METHYL-(4-O-TOLYLOXY-CHROMAN-3-YLMETHYL)-AMINE

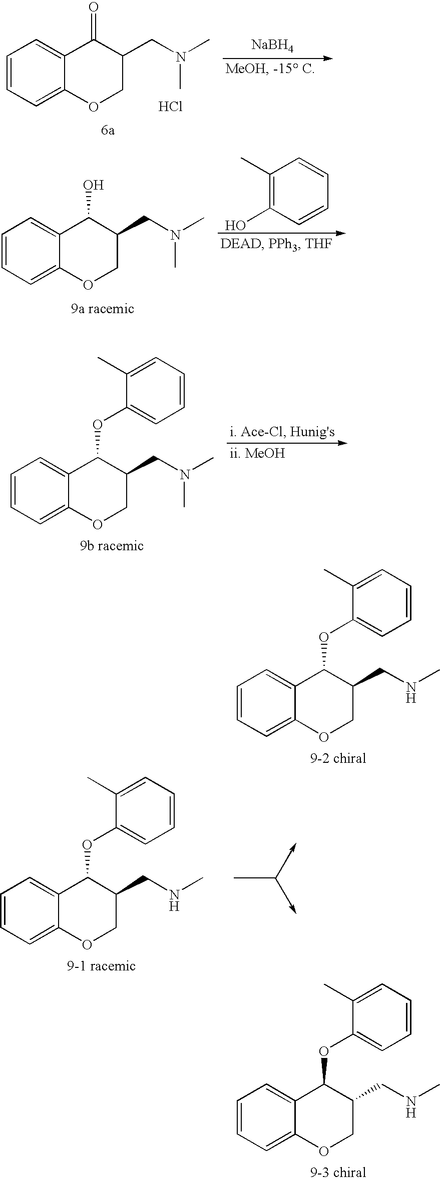 Step 9A:

To a stirred solution of the ketone 6a (12.26 g, 50.7 mmol) in methanol (500 mL) at −15° C. internal reaction temperature was added sodium borohydride (2.12 g, 55.7 mmol) portionwise. Upon completion the reaction mixture was allowed to warm to room temperature and concentrated in vacuo. The residue was partitioned between distilled water (100 mL) and ethyl acetate (100 mL), and the aqueous phase was extracted with ethyl acetate (3×100 mL). The combined organics were dried over magnesium sulfate, filtered and concentrated in vacuo. The residue was purified by flash chromatography eluting with 0 to 7% methanol in dichloromethane to afford 9a as a colorless viscous oil (7.40 g, 70%) ¹H NMR (300 MHz, CDCl₃) δ 7.45 (dt, J=7.8, 1.5 Hz, 1H), 7.15 (m, 1H), 6.95 (dt, J=7.8, 1.5 Hz, 1H), 6.78 (dd, J=8.1, 1.2 Hz, 1H), 4.76 (d, J=8.7 Hz, 1H), 4.09 (dd, J=11.0, 3.6 Hz, 1H), 3.79 (t, J=11.0 Hz, 1H), 2.42 (m, 2H), 2.34 (s, 6H), 2.27 (m, 1H). APCI MS m/e: 208.0 ([M+H]⁺).

Step 9B:

To a solution of amine 9a (5.87 g, 28.4 mmol) in THF (200 mL) was added o-methyl phenol (5.9 mL, 56.7 mmol) and triphenylphosphine (9.95 g, 34.0 mmol). To this solution was added diethylazodicarboxylate (DEAD, 5.4 mL, 34.0 mmol). The resultant mixture was allowed to stir for 16 h at room temperature, then concentrated in vacuo and purified by flash chromatography eluting with 0 to 20% ethyl acetate in hexanes to afford the amine 9b as a pale yellow oil (3.82 g, 45%). ¹H NMR (300 MHz, CDCl₃) δ 7.20 (m, 5H), 6.90 (m, 3H), 5.21 (s, 1H), 4.37 (dd, J=10.8, 2.4 Hz, 1H), 4.17 (ddd, J=10.8, 8.7, 1.5 Hz, 1H), 2.29 (m, 3H), 2.22 (s, 6H), 2.12 (s, 3H). APCI MS m/e: 298.1 ([M+H]⁺).

Step 9C:

To a stirred solution of amine 9b (2.25 g, 7.6 mmol) in 1,2-dichloroethane (50 mL) was added diisopropylethylamine (2.5 mL, 15.2 mmol), followed by 1-chloroethylchloroformate (4.1 mL, 38.0 mmol). The reaction mixture was heated at 40° C. for 30 min then cooled to room temperature, diluted with dichloromethane (100 mL), washed with distilled water (100 mL) and saturated aqueous sodium carbonate (100 mL), dried over magnesium sulfate, filtered and concentrated in vacuo. Methanol (60 mL) was added to the residue that was made basic (pH 8.0 with universal indicator paper) by the dropwise addition of 2M ammonia in methanol. This solution was stirred at room temperature for 3 h then concentrated in vacuo and purified by flash chromatography to afford methyl-(4-o-tolyloxy-chroman-3-ylmethyl)-amine 9-1 as a pale yellow oil (0.75 g, 35%). ¹H NMR (300 MHz, CDCl₃) δ 7.20 (m, 5H), 6.89 (m, 3H), 5.27 (s, 1H), 4.44 (dd, J=11.4, 2.4 Hz, 1H), 4.17 (ddd, J=11.4, 2.7, 1.5 Hz, 1H), 2.74 (dd, J=12.3, 7.2 Hz, 1H), 2.65 (dd, J=12.3, 7.2 Hz, 1H), 2.47 (m, 4H), 2.09 (s, 3H). APCI MS m/e: 284.0 ([M+H]⁺).

Step 9D:

The enantiomerically pure compounds, 9-2 and 9-3 (250 mg each, 67% recovery), were obtained from 9-1 (750 mg) by chiral preparative HPLC using a Chiralcel OD-H (20×250 cm) column eluting with 9:1 hexanes/ethanol with 0.1% diethylamine at a flow rate of 15 mL/min.

### Example 10

### DIMETHYL-(4-O-TOLYLOXY-THIOCHROMAN-3-YLMETHYL)-AMINE

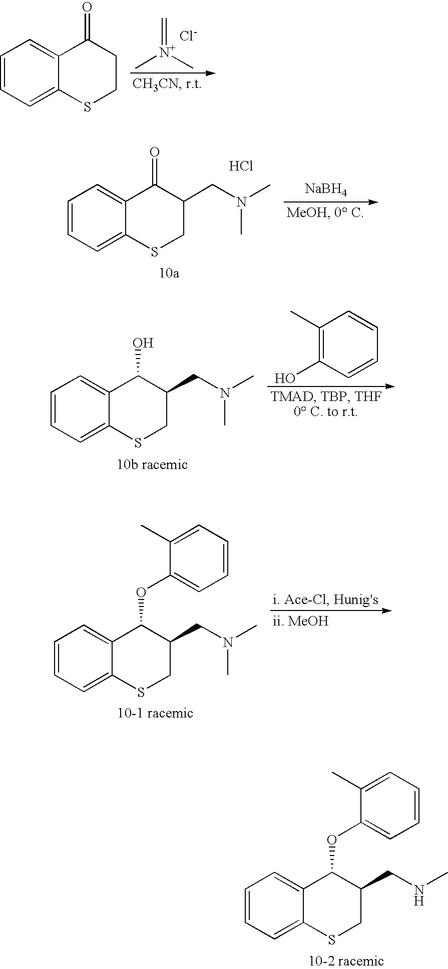 Step 10A:

To a solution of thiochroman-4-one (5.00 g, 30.5 mmol) in dry acetonitrile (50 mL) was added N,N-dimethylmethyleneammonium chloride (2.85 g, 30.5 mmol). The resultant solution was stirred vigorously for 16 h. The precipitate was filtered under reduced pressure and washed with diethyl ether to obtain 10a as a white solid (1.80 g, 23%) that was used without further purification. APCI MS m/e: 222.0 ([M+H]⁺).

Step 10B:

To a stirred solution of the ketone 10a (550 mg, 2.5 mmol) in methanol (10 mL) at 0° C. was added sodium borohydride (105 mg, 2.7 mmol) in distilled water (1 mL). The reaction mixture was stirred for 30 min then warmed to room temperature and concentrated in vacuo. The residue was partitioned between water (15 mL) and ethyl acetate (25 mL), and the aqueous phase was extracted with ethyl acetate (25 mL). The combined organics were dried over magnesium sulfate, filtered and concentrated in vacuo. The residue was purified by flash chromatography eluting with 0 to 4% methanol in dichloromethane to afford 10b as an off white solid (110 mg, 20%). ¹H NMR (300 MHz, CDCl₃) δ 7.64 (m, 1H), 7.11 (m, 3H), 4.67 (dd, J=9.3, 0.9 Hz, 1H), 2.78 (dd, J=12.6, 10.2 Hz, 1H), 2.68 (dd, J=12.6, 4.2 Hz, 1H), 2.66 (dd, J=12.0, 11.4 Hz, 1H), 2.47 (dd, J=12.0, 3.3 Hz, 1H), 2.34 (s, 6H), 2.23 (m, 1H). APCI MS m/e: 224.0 ([M+H]⁺).

Step 10C:

To a solution of 10b (110 mg, 0.5 mmol) in THF (3 mL) was added o-methyl phenol (52 μL, 0.5 mmol) and tri-n-butylphosphine (185 μL, 0.75 mmol). The mixture was cooled to 0° C. and 1,1′-azobis(N,N-dimethylformamide) (TMAD, 130 mg, 0.75 mmol) was added. The resultant mixture was allowed to gradually warm to room temperature and stirred for a further 16 h. The reaction mixture was concentrated in vacuo and purified by flash chromatography eluting with 0 to 2% methanol in dichloromethane to afford dimethyl-(4-o-tolyloxy-thiochroman-3-ylmethyl)-amine 10-1 as a pale yellow oil (118 mg, 76%). ¹H NMR (300 MHz, CDCl₃) δ 6.94-7.20 (m, 7H), 6.89 (td, J=7.2, 0.9 Hz, 1H), 5.17 (d, J=3.9 Hz, 1H), 3.58 (dd, J=12.3, 3.6 Hz, 1H), 2.85 (dd, J=12.3, 3.6 Hz, 1H), 2.68 (app. octet, J=3.9 Hz, 1H), 2.40 (dd, J=12.6, 7.8 Hz, 1H), 2.24 (s, 6H), 2.20 (dd, J=12.6, 7.8 Hz, 1H). APCI MS m/e: 314.0 ([M+H]⁺).

Step 10D:

To a stirred solution of 10-1 (109 mg, 0.35 mmol) in 1,2-dichloroethane (6 mL) was added diisopropylethylamine (115 μL, 0.70 mmol), followed by 1-chloroethylchloroformate (376 μL, 3.50 mmol). The reaction mixture was heated at 40° C. for 1 h then cooled to room temperature. Methanol (6 mL) was added to the reaction mixture and heating continued for a further 1 h. The reaction mixture was concentrated in vacuo and purified by prep HPLC to afford 10-2 as a white solid (65 mg, 63%). ¹H NMR (300 MHz, CDCl₃) δ 6.88-7.22 (m, 8H), 5.26 (m, 1H), 3.79 (m, 1H), 3.11 (m, 1H), 2.97 (m, 3H), 2.73 (s, 3H), 1.99 (s, 3H). APCI MS m/e: 300.0 ([M+H]⁺).

### Example 11

### (6-FLUORO-4-O-TOLYLOXY-CHROMAN-3-YLMETHYL)-METHYL-AMINE

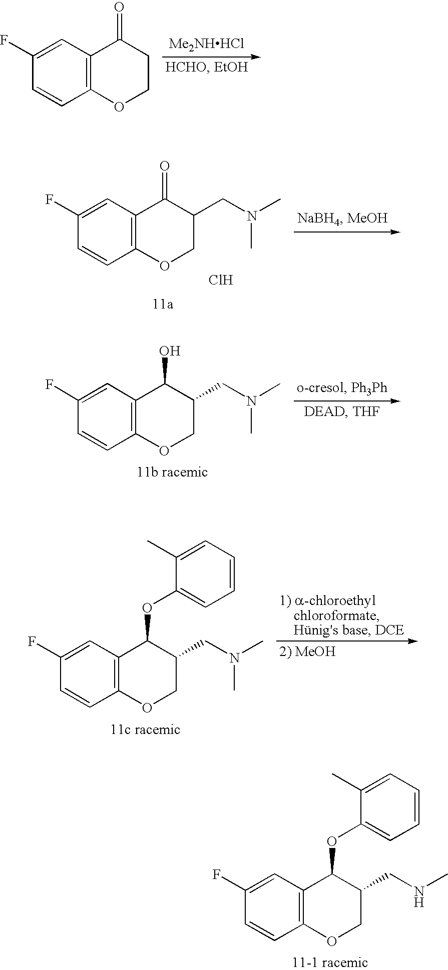 Step 11A:

A mixture of 6-fluorochromanone (2.00 g, 12.0 mmol), dimethylamine hydrochloride (2.9 g, 36.0 mmol) and paraformaldehyde (1.8 g, 60.0 mmol) was suspended in ethanol (48 mL). To this mixture hydrochloric acid (12 N, 0.5 mL) was added, and the mixture was heated to reflux. After 7 hours, the reaction mixture was removed from heat and allowed to cool. The resulting white precipitate was filtered and washed with acetone to give 11a as the hydrochloride salt (1.14 g, 37%).

¹H NMR (300 MHz, CDCl₃) δ 7.51 (d, J=8.1, 3.3 Hz, 1H), 7.20-7.33 (m, 1H), 7.01 (dd, J=9.2, 4.3 Hz, 1H), 5.18 (dd, J=11.4, 5.4 Hz, 1H), 4.35 (app t, J=11.7 Hz, 1H), 3.48-3.63 (m, 1H), 2.82-3.10 (m, 1H), 2.94 (d, J=4.8 Hz, 3H), 2.89 (d, J=4.8 Hz, 3H), 2.73 (app t, J=5.7 Hz, 1H). ESI MS m/e: 224.2 ([M+H]⁺)

Step 11B:

Ketone 11a (1.14 g, 4.4 mmol) was suspended in methanol (22 mL) and cooled in a salt/ice bath to −10° C. Sodium borohydride (181 mg, 4.8 mmol) was added at once to this suspension. After one hour, the reaction mixture was reduced under vacuum and partitioned between water and ethyl acetate (30 mL each). The organic layer was separated, dried over magnesium sulfate and evaporated. This crude material was chromatographed with 5% methanol in dichloromethane to yield the desired trans aminoalcohol 11b as a colorless oil which solidified on standing (670 mg, 68%).

¹H NMR (300 MHz, CDCl₃) δ 7.19 (ddd, J=9.3, 3.3, 1.2 Hz, 1H), 6.81-6.88 (m, 1H), 6.72 (dd, J=9.0, 4.5 Hz, 1H), 4.73 (d, J=8.7 Hz, 1H), 4.07 (dd, J=11.1, 3.6 Hz, 1H), 3.75 (app t, J=11.1 Hz, 1H), 2.35 (s, 6H), 2.22-2.52 (m, 3H). APCI MS m/e: 226.0 ([M+H]⁺)

Step 11C:

To the solution of the alcohol 11b (0.67 g, 3.0 mmol) in THF (15 mL) at room temperature, o-cresol (0.62 mL, 6.0 mmol) and triphenylphosphine (944 mg, 3.6 mmol) were added. Diethyl azodicarboxylate (627 mg, 3.6 mmol) was added last via syringe and the mixture was stirred overnight under an atmosphere of nitrogen. Excess THF was evaporated under reduced pressure and the residue was chromatographed with 10-15% ethyl acetate in hexanes. Compound 11c was isolated as a clear oil (730 mg, 77%).

¹H NMR (300 MHz, CDCl₃) δ 7.13-7.22 (m, 3H), 6.81-6.99 (m, 4H), 5.18 (s, 1H), 4.33 (dd, J=11.1, 2.4 Hz, 1H), 4.09-4.17 (m, 1H), 2.16-2.31 (m, 3H), 2.22 (s, 6H), 2.13 (s, 3H). APCI MS m/e: 316.0 ([M+H]⁺)

Step 11D:

To the solution of the dimethylamine 11c (284 mg, 0.9 mmol) and Hünig's base (0.44 mL, 2.7 mmol) in 1,2-dichloroethane (4.5 mL), 1-chloroethylchloroformate (0.97 nL, 9.0 mmol) was added and the resulting yellow solution was heated at 40° C. until the conversion was complete. Methanol (1.0 mL) was added and the mixture was stirred at 40° C. for 2 hours until none of the intermediate could be observed by HPLC/MS. The solvent was evaporated under reduced pressure and one fourth of the residue purified on a prep. HPLC/MS to afford (6-fluoro-4-o-tolyloxy-chroman-3-ylmethyl)-methyl-amine 11-1 as a trifluoroacetate salt (6.4 mg, 7% yield).

¹H NMR (300 MHz, CDCl₃) δ 9.89 (br s, 1H), 9.69 (br s, 1H), 6.75-7.26 (m, 6H), 6.75 (dd, J=8.4, 3.0 Hz, 1H), 5.13 (br s, 1H), 4.43 (dd, J=15.6, 2.1 Hz, 1H), 4.31 (d, J=11.4 Hz, 1H), 2.0-2.95 (m, 2H), 2.64-2.68 (m, 1H), 2.59 (s, 3H), 2.01 (s, 3H). ESI MS m/e: 302.2 ([M+H]⁺)

### Example 12

METHYL-(4-O-TOLYLOXY-ISOCHROMAN-3-YLMETHYL)-AMINE 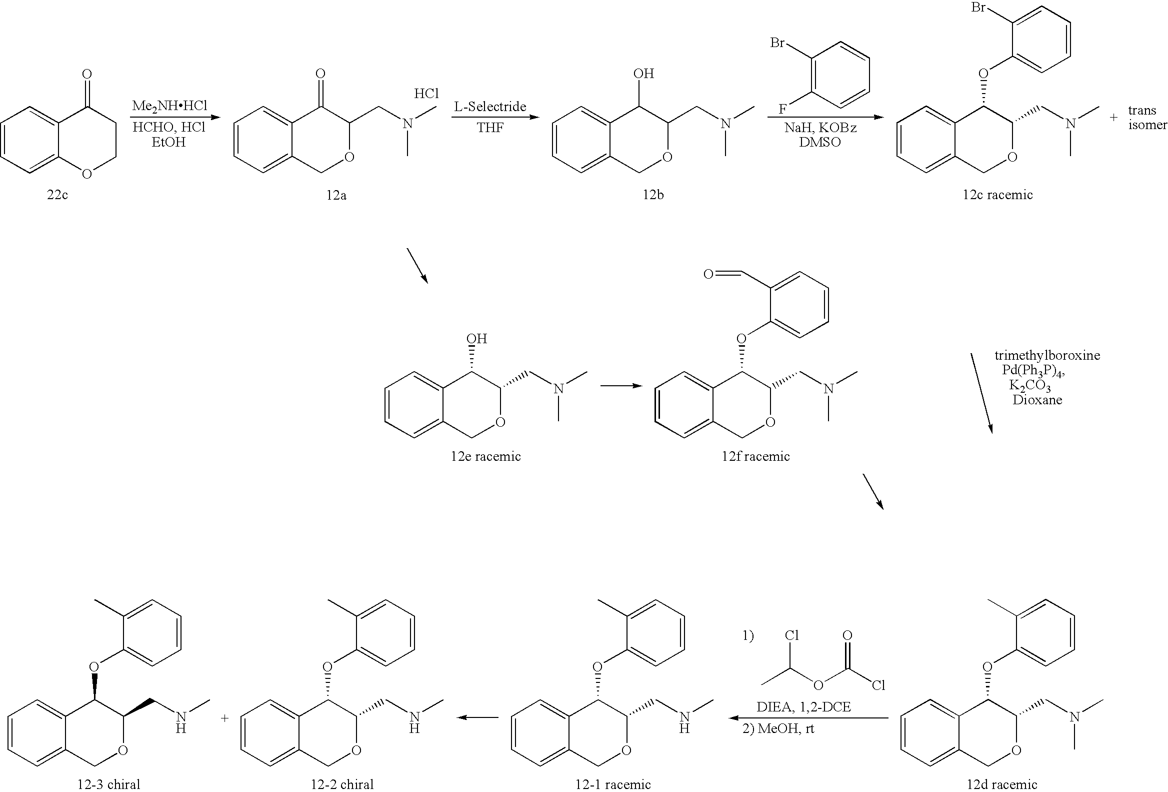
Step 12A:

To the solution of 1H-isochromen-4(3H)-one 22c (24.0 g, 0.16 mol) in ethanol (500 mL), dimethylamine hydrochloride (66.2 g, 0.81 mole), paraformaldehyde (14.6 g, 0.49 mole) and aqueous hydrochloric acid (12N, 13.5 mL) were added. The cloudy suspension was heated at reflux overnight. After cooling, excess solvent was removed and the residue recrystalized from isopropyl alcohol twice. The resulting off-white precipitate was filtered and dried to give 12a (29.5 g, 76%).

¹H NMR (300 MHz, CDCl₃) δ 8.02 (d, J=7.8 Hz, 1H), 7.62 (dt, J=7.8, 1.5 Hz, 1H), 7.44 (t, J=7.5 Hz, 1H), 7.23 (d, J=7.8 Hz, 1H), 5.24-5.28 (m, 1H), 5.21 (d, J=15.3 Hz, 1H), 4.99 (d, J=15.3 Hz, 1H), 4.03 (dd, J=14.4, 2.4 Hz, 1H), 3.17-3.25 (m, 1H), 3.02 (s, 3H), 2.97 (s, 3H). ESI MS m/e: 206.0 ([M+H]⁺)

Step 12B:

To the suspension of ketone 12a (27.0 g, 112 mmol) in THF (600 mL) at −30° C. (internal temperature), L-Selectride (1.0 M in THF, 224 mL, 224 mmol) was added drop-wise over one hour. The reaction mixture was allowed to warm to 0° C. and was diluted with ethyl acetate (500 mL). The organic layer was washed successively with 10% sodium hydroxide solution (500 mL), water (500 mL) and brine (500 mL), followed by extraction with a 1 N hydrochloric acid solution (2×500 mL). The 1N hydrochloric acid solution extracts were combined, taken to pH 9 with sodium hydroxide (50% weight, 75 mL) and extracted with ethyl acetate (4×500 mL). The combined organic extracts were dried over sodium sulfate, filtered and concentrated to give 12b as a 1.4:1.0 mixture of cis and trans isomers respectively (14.5 g, 62%).

APCI MS m/e: 208.0 ([M+H]⁺)

Step 12C:

To a suspension of sodium hydride (2.21 g, 55.2 mmol) in dry DMSO (125 mL) was added 12b (7.62 g, 36.8 mmol) and the mixture stirred for 15 minutes. Potassium benzoate (5.9 g, 36.8 mmol) was added. After 15 minutes 1-bromo-2-fluorobenzene (8.0 mL, 73.6 mmol) was added and the mixture was heated at 65° C. overnight. The reaction mixture was diluted with ethyl acetate (500 mL) and washed successively with saturated bicarbonate solution (300 mL) and brine (300 mL). After drying over sodium sulfate, the solvent was removed and the residue was chromatographed on silica gel using 3% methanol in dichloromethane to give the cis isomer 12c (5.2 g, 39%).

¹H NMR (300 MHz, CDCl₃) δ 7.51 (dd, J=4.5, 0.9 Hz, 1H), 7.28 (dt, J=4.5, 0.6 Hz, 1H), 7.21 (dt, J=5.1, 1.2 Hz, 1H), 7.07-7.10 (m, 3H), 6.93 (d, J=4.5 Hz, 1H), 6.88 (dt, J=4.5, 0.9 Hz, 1H), 5.25 (d, J=1.2 Hz, 1H), 5.09 (d, J=9.3 Hz, 1H), 4.88 (d, J=9.8 Hz, 1H), 4.00 (ddd, J=4.2, 3.3, 1.2 Hz, 1H), 2.85 (dd, J=7.8, 4.2 Hz, 1H), 2.81 (dd, J=8.1, 3.3 Hz, 1H), 2.31 (s, 6H). APCI MS m/e: 361.9 ([M+H]⁺)

Step 12D:

To a solution of 12c (5.1 g, 14.1 mmol) in dioxane (45 mL) was added trimethylboroxine (50% wt in THF, 3.9 mL, 15.5 mmol) and potassium carbonate (5.3 g, 38.4 mmol). The mixture was degassed with nitrogen for fifteen minutes. After the addition of palladium tetrakistriphenylphosphine (1.2 g, 1.04 mmol) the mixture was degassed with nitrogen for another 15 minutes. The reaction mixture was heated in a sealed vessel at 110° C. for 2 hours. After cooling, the suspension was filtered through a plug of celite, washing with ethanol (3×100 mL). The filtrate was evaporated and redissolved in 20% ethyl acetate in hexanes. The resulting precipitate was removed by filtration and the filtrate was concentrated under vacuum. Silica gel chromatography using 30% tetrahydrofuran in hexanes, containing 2% triethylamine, gave 12d (90% pure, 2.8 g, 67% yield). APCI MS m/e: 298.1 ([M+H]⁺)

Alternate Step 12D:

To a stirred suspension of ketone 12a (350 g, 1.45 mol) in dichloromethane (3 L) at −60° C. (dry ice/acetone bath, internal reaction temperature) under a nitrogen atmosphere was added a slow stream of LS-selectride® (1M in tetrahydrofuran, 3.3 L, 3.3 mol) maintaining the internal temperature between −55 and −62° C. The mixture was stirred at that temperature for a further 30 mins then the cold bath was removed and the mixture was allowed to warm to −45° C. The reaction was quenched by the dropwise addition of 1M aqueous sodium hydroxide (2 L) during which time the reaction temperature rose to 0° C. The mixture was concentrated in vacuo and extracted with ethyl acetate (3L then 2×1 L), the extracts were dried over magnesium sulfate, filtered and concentrated in vacuo. The residue was redissolved in ethyl acetate (4 L) and extracted with 1M aqueous hydrochloric acid (2×1 L). The aqueous phase was basified (pH 11) by the addition of 50% aqueous sodium hydroxide and back extracted with ethyl acetate (3×1 L), the organics were dried over magnesium sulfate, filtered and concentrated in vacuo. The resulting yellow solid was dried in a vacuum oven at ambient temperature for 60 h to afford 12e as a yellow solid having a 8.5:1.0 mixture of cis and trans isomers respectively (260 g, 87%). ¹H NMR (300 MHz, CDCl₃) δ 7.43 (m, 1H), 7.27 (m, 2H), 7.03 (m, 1H), 4.90 (d, J=15.0 Hz, 1H), 4.75 (d, J=15.0 Hz, 1H), 4.58 (d, J=2.0 Hz, 1H), 3.72 (ddd, J=6.0, 4.5 and 2.0 Hz, 1H), 2.84 (dd, J=13.2 and 6.0 Hz, 1H), 7.24 (dd, J=13.2 and 4.5 Hz, 1H), 2.35 (s, 6H). APCI MS m/e: 208.1 ([M+H]⁺).

Alcohol 12e was converted to 12f following the procedure for step 7A except potassium bis(trimethylsilyl)amide was used in place of sodium hydride and potassium benzoate for large scale reactions. ¹H NMR (300 MHz, CDCl₃) δ 10.00 (d, J=0.9 Hz, 1H), 7.78 (dd, J=7.8 and 1.8 Hz, 1H), 7.62 (ddd, J=8.4, 7.2 and 1.8 Hz, 1H), 7.44 (d, J=8.4 Hz, 1H), 7.28 (m, 2H), 7.11 (m, 2H), 6.91 (d, J=6.9 Hz, 1 H), 5.29 (d, J=1.4 Hz, 1H), 5.05 (d, J=15.8 Hz, 1H), 4.88 (d, J=15.8 Hz, 1H), 4.03 (td, J=6.6 and 1.8 Hz, 1H), 2.82 (dd, J=12.8 and 6.6 Hz, 1H), 2.72 (dd, J=12.8 and 6.6 Hz, 1H), 2.33 (s, 6H).

APCI MS m/e: 312.1 ([M+H]⁺).

Aldehyde 12f was converted to amine 12d following the procedures for steps 7B through D. ¹H NMR (300 MHz, CDCl₃) δ 7.27 (dd, J=7.5 and 1.2 Hz, 1H), 7.12 (m, 2H), 7.07 (m, 3H), 6.93 (m, 2H), 5.15 (d, J=2.1 Hz, 1H), 5.05 (d, J=15.3 Hz, 1H), 4.88 (d, J=15.3 Hz, 1H), 4.03 (ddd, J=6.9, 4.8 and 2.1 Hz, 1H), 2.83 (dd, J=13.3 and 6.9 Hz, 1H), 2.77 (dd, J=13.3 and 4.8 Hz, 1H), 2.35 (s, 6H), 1.93 (s, 3H). APCI MS m/e: 312.1 ([M+H]⁺).

Step 12E:

A solution of the dimethylamine 12d (2.8 g, 9.4 mmol) in 1,2-dichloroethane (40 mL) was treated with N,N-diisopropyl ethylamine (3.3 mL, 18.8 mmol), proton sponge (catalytic amount) and 1-chloroethylchloroformate (2.05 mL, 18.8 mmol). The reaction mixture was heated at 40° C. for an hour and allowed to cool. After evaporation under reduced pressure the residue was redissolved in methanol (40 mL) and was stiredr at room temperature for 2 hours. The solvent was removed under reduced pressure and the material taken up in dichloromethane (200 mL). This solution was washed with saturated sodium bicarbonate solution (50 mL) followed by brine (50 mL). The organic layer was dried over sodium sulfate and concentrated to give a yellow oil, which on silica gel chromatography (7% methanol in dichloromethane) produced 12-1 (90% pure, 1.5 g, 56%).

¹H NMR (300 MHz, CDCl₃) δ 7.28 (d, J=4.0 Hz, 1H), 7.18 (t, J=4.8 Hz, 1H), 7.07-7.11 (m, 4H), 6.98 (d, J=4.5 Hz, 1H), 6.92 (t, J=4.1 Hz, 1H), 5.16 (d, J=1.2 Hz, 1H), 5.02 (d, J=9.1 Hz, 1H), 4.85 (d, J=9.1 Hz, 1H), 4.04-4.07 (m, 1H), 3.15 (dd, J=7.5, 5.2 Hz, 1H), 2.95 (dd, J=7.5, 2.4 Hz, 1H), 2.50 (s, 3H), 2.01-2.05 (m, 2H), 1.97 (s, 3H). APCI MS m/e: 284.0 ([M+H]⁺)

Step 12F:

A chiral HPLC column (OJ-H column, 15.0 mL/minute flow, 95:5 Hexane to ethanol, 0.1% diethylamine) was used to resolve 12-1 to the individual enantiomers: 12-2 (613 mg, 87%) and 12-3 (477 mg, 68%).

The following compounds were made according to the above procedure (skipping Step 12D for compounds 12-4 to 12-13).

| | |
|---|---|
| | racemic or chiral |
| 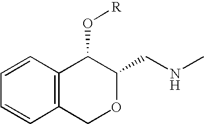 | |

| No. | R | Stereochem | MW | MH⁺ | t_{R} (method) |
|---|---|---|---|---|---|
| 12-1 | 2-CH₃-phenyl | R,R and S,S | 283.4 | 284.0 | 4.74 (2) |
| 12-2 | 2-CH₃-phenyl | S,S | 283.4 | 284.0 | 16.08 (3) |
| 12-3 | 2-CH₃-phenyl | R,R | 283.4 | 284.0 | 4.73 (2) |
| 12-4 | 2-CF₃-phenyl | R,R and S,S | 337.3 | 338.0 | 4.80 (2) |
| 12-5 | 2-CF₃-phenyl | R,R | 337.3 | 338.0 | 7.41 (5) |
| 12-6 | 2-CF₃-phenyl | S,S | 337.3 | 338.2 | 7.31 (5) |
| 12-7 | 2-Me-3-F-phenyl | S,S | 301.4 | 301.8 | 7.14 (5) |
| 12-8 | 2-Br-phenyl | S,S | 348.2 | 347.9 | 4.22 (2) |
| 12-9 | 2-Br-phenyl | R,R | 348.2 | 347.9 | 4.17 (2) |
| 12-10 | 2-CF₃-6-F-phenyl | S,S and R,R | 355.3 | 356.0 | 4.46 (2) |
| 12-11 | 2-F-phenyl | S,S and R,R | 287.3 | 288.0 | 3.89 (2) |
| 12-12 | 2-CF₃-6-F-phenyl | R,R | 355.3 | 356.0 | 4.58 (6) |
| 12-13 | 2-CF₃-6-F-phenyl | S,S | 355.3 | 356.0 | 4.62 (6) |

### EXAMPLE 13

### DIMETHYL-(4-O-TOLYLOXY-ISOCHROMAN-3-YLMETHYL)-AMINE

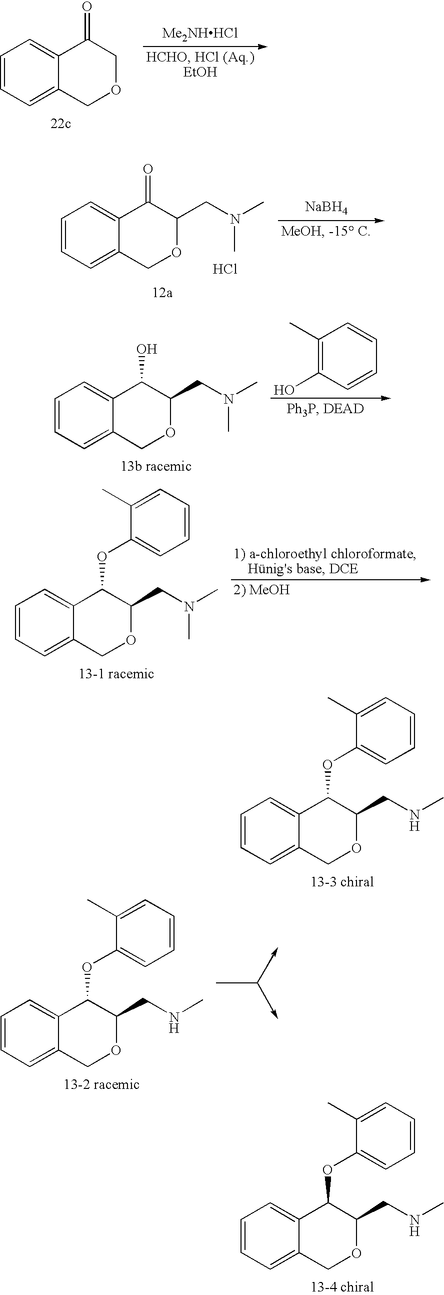 Step 13A:

To the solution of the 1H-isochromen-4(3H)-one 22a (49.5 g, 334 mmol) in ethanol (1 L), dimethylamine hydrochloride (136 g, 1.67 mole), paraformaldehyde (30 g, 1.00 mole) and aqueous hydrochloric acid (12N, 21 mL) were added. The cloudy suspension was heated at reflux overnight. After cooling, the solution was concentrated to half original volume under reduced pressure. The resulting off-white precipitate (40 g, 50%) was filtered and washed with ether to give 12a. A second batch of product (20 g, 25%) was obtained from the mother liquor by treatment with ether.

¹H NMR (300 MHz, CDCl₃) δ 8.02 (d, J=7.8 Hz, 1H), 7.62 (dt, J=7.8, 1.5 Hz, 1H), 7.44 (t, J=7.5 Hz, 1H), 7.23 (d, J=7.8 Hz, 1H), 5.24-5.28 (m, 1H), 5.21 (d, J=15.3 Hz, 1H), 4.99 (d, J=15.3 Hz, 1H), 4.03 (dd, J=14.4, 2.4 Hz, 1H), 3.17-3.25 (m, 1H), 3.02 (s, 3H), 2.97 (s, 3H). ESI MS m/e: 206.0 ([M+H]⁺)

Step 13B:

The ketone 12a (41 g, 170 mmol) was dissolved in methanol (850 mL) and cooled in an ice/salt bath. Sodium borohydride was added slowly such that the internal temperature would not rise above −10° C. After completion, the reaction mixture was allowed to warm up to room temperature. The excess solvent was evaporated under reduced pressure and the residue partitioned between water and ethyl acetate (300 mL each). The aqueous layer was extracted with more ethyl acetate (200 mL) and the combined organic layers dried over magnesium sulfate. On evaporation, 13b was obtained as an off-white solid (30.2 g), which was used without further manipulation.

¹H NMR (300 MHz, CDCl₃) δ 7.60 (d, J=7.5 Hz, 1H), 7.20-7.31 (m, 2H), 6.98 (d, J=7.5 Hz, 1H), 4.88 (d, J=15.0 Hz, 1H), 4.80 (d, J=14.7 Hz, 1H), 3.57 (ddd, J=9.6, 8.7, 4.8 Hz, 1H), 2.67-2.80 (m, 2H), 2.38 (s, 3H), 2.37 (s, 3H). ESI MS m/e: 208.0 ([M+H]⁺). The crude material also contained 19% of the cis alcohol according to NMR.

Step 13C:

The alcohol 13b (29.6 g, 0.14 mmol), o-cresol (30 g, 0.28 mmol) and triphenylphosphine (44g, 0.17 mmol) were dissolved in anhydrous THF (700 mL). To this mixture, diethylazodicarboxylate (26.7 mL, 0.17 mmol) was added at room temperature and the mixture was stirred overnight. Excess solvent was then removed under reduced pressure. One third of the crude material was purified on a silica gel column eluting with 50-70% ethyl acetate in hexanes. Dimethyl-(4-o-tolyloxy-isochroman-3-ylmethyl)-amine 13-1 was thus obtained as a mixture with triphenylphosphine oxide. Repeated trituration with ether and filtration of the resulting white solid was used to remove some of the triphenylphosphine oxide. This compound 13-1 (7.2 g) was carried on to the next step.

¹H NMR (300 MHz, CDCl₃) δ 7.35 (d, J=7.2 Hz, 1H), 7.17-7.29 (m, 4H), 7.04-7.09 (m, 2H), 6.94 (t, J=6.9 Hz, 1H), 5.28 (d, J=7.8 Hz, 1H), 4.96 (d, J=15.3 Hz, 1H), 4.89 (d, J=15.0 Hz, 1H), 4.00 (dt, J=8.1, 2.4 Hz, 1H), 2.59 (dd, J=13.2, 8.4 Hz, 1H), 2.49 (dd, J=13.2, 2.4 Hz, 1H), 2.30 (s, 6H), 2.24 (s, 3H). ESI MS m/e: 298.0 ([M+H]⁺)

Step 13D:

To the solution of the dimethylamine 13-1 (7.2 g, 2.29 mmol) in dichloroethane (100 mL), N,N-diisopropyl ethylamine (Hünig's base, 7.92 mL, 48.0 mmol) and 1-chloroethylchloroformate (5.23 mL, 48.0 mmol) were added. The reaction was allowed to proceed for 24 hours under an atmosphere of nitrogen. When all the starting material was consumed, methanol (15 mL) was added and the mixture was heated at 40° C. for 3 hours. Saturated sodium bicarbonate (200 mL) and dichloromethane (200 mL) were added to the reaction mixture. The organic layer was dried over magnesium sulfate and evaporated under reduced pressure. Silica gel chromatography using 2-8% methanol in dichloromethane gave pure 13-2 (2.1 g, 6% from 12a).

¹H NMR (300 MHz, CDCl₃) δ 9.70 (br s, 1H), 9.25 (br s, 1H), 7.16-7.31 (m, 5H), 6.92-7.01 (m, 3H), 5.25 (d, J=8.4 Hz, 1H), 4.89 (d, J=15.6 Hz, 1H), 4.82 (d, J=15.6 Hz, 1H), 4.18 (dt, J=9.0, 2.1 Hz, 1H), 3.30-3.34 (m, 1H), 3.04-3.07 (m, 1H), 2.74 (s, 3H), 2.19 (s, 3H). ESI MS m/e: 284.0 ([M+H]⁺)

Step 13F:

The enantiomerically pure compounds, 13-3 (122 mg, 77% recovery) and 13-4 (75 mg, 47% recovery) were obtained from 13-2 (350 mg, trifluoroacetate salt) by chiral preparative HPLC using a Chiralcel AD-H column eluting with 85:15 hexanes/ethanol with 0.1% diethylamine at a flow rate of 15 mL/min.

### Example 14

### METHYL-[4-(NAPHTHALEN-1-YLOXY)-ISOCHROMAN-3-YLMETHYL]-AMINE

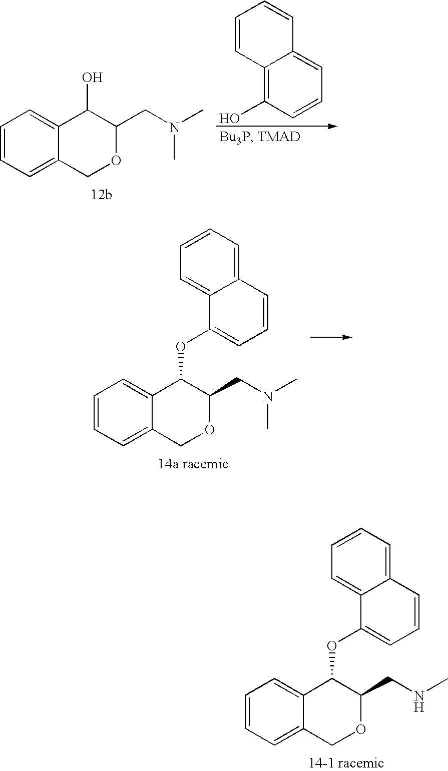 Step 14A:

To a solution of alcohol 12b (250 mg, 1.2 mmol) in tetrahydrofuran (5 mL) at room temperature was added 1-naphthol (350 mg, 2.4 mmol), followed by tributylphosphine (0.45 mL, 1.8 mmol). 1,1′-Azobis-N,N-dimethylformamide (310 mg, 1.8 mmol) was added to this pale yellow solution in two portions over one minute and the mixture was stirred at room temperature overnight. After concentrating the reaction mixture in vacuo, the residue was purified via flash column chromatography with 5-10% methanol in dichloromethane to yield the trans product 14a as a mixture with tributylphosphine oxide. APCI MS m/e: 320.1 ([M+H]⁺)

Step 14B:

Compound 14a was demethylated as described in Step 13D to give methyl-[4-(naphthalen-1-yloxy)-isochroman-3-ylmethyl]-amine 14-1 (96 mg, 25% over two steps).

¹H NMR (300 MHz, CDCl₃) δ 8.27 (d, J=4.5 Hz, 1H), 7.84 (d, J=4.7 Hz, 1H), 7.38-7.53 (m, 5H), 7.25-7.27 (m, 1H), 7.16-7.19(m, 2H), 7.06 (d, J=4.5 Hz, 1H), 5.64 (d, J=5.3 Hz, 1H), 5.01 (d, J=9.1 Hz, 1H), 4.92 (d, J=9.1 Hz, 1H), 4.17 (dt, J=5.1, 1.6 Hz, 1H), 3.03 (dd, J=7.5, 1.6 Hz, 1H), 2.81 (dd, J=7.5, 4.7 Hz, 1H), 2.45 (s, 3H).

APCI MS m/e: 320.1 ([M+H]⁺)

The following compounds were made according to this procedure:

| | |
|---|---|
| | racemic or chiral |
| 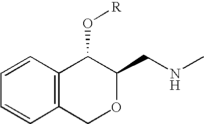 | |

| No. | R | Stereochem | MW | MH⁺ | t_{R} (method) |
|---|---|---|---|---|---|
| 14-1 | 1-naphthyl | R,S and S,R | 319.4 | 320.1 | 20.88 (3) |
| 14-2 | 2-methoxy-phenyl | R,S and S,R | 299.4 | 300.1 | 16.11 (3) |
| 14-3 | 2-ethoxy-phenyl | R,S and S,R | 313.4 | 314.1 | 17.91 (3) |
| 14-4 | 2,2-dimethyl-2,3-di- | R,S and S,R | 339.4 | 340.2 | 19.60 (3) |
| | hydro-benzofuran-7-yl | | | | |
| 14-5 | Benzofuran-7-yl | R,S and S,R | 309.4 | 310.1 | 17.16 (3) |
| 14-6 | Benzofuran-4-yl | R,S and S,R | 309.4 | 310.1 | 17.54 (3) |
| 14-7 | Benzo[b]thiophen-7-yl | R,S and S,R | 325.4 | 326.1 | 19.05 (3) |
| 14-8 | Benzo[b]thiophen-4-yl | R,S and S,R | 325.4 | 326.1 | 19.47 (3) |
| 14-9 | 2-CH₃S-phenyl | R,S and S,R | 315.4 | 316.0 | 4.89 (2) |
| 14-10 | Benzo[b]thiophen-7-yl | R,S | 325.4 | 326.1 | 18.75 (3) |
| 14-11 | Benzo[b]thiophen-7-yl | S,R | 325.4 | 326.1 | 18.53 (3) |
| 14-12 | Benzo[b]thiophen-4-yl | S,R | 325.4 | 326.1 | 19.37 (3) |
| 14-13 | Benzo[b]thiophen-4-yl | R,S | 325.4 | 326.1 | 19.37 (3) |
| 14-14 | 2,4-dimethyl-phenyl | R,S and S,R | 297.4 | 298.1 | 5.25 (2) |
| 14-15 | 2,4-dimethyl-phenyl | R,S | 297.4 | 298.1 | 20.02 (3) |
| 14-16 | 2,4-dimethyl-phenyl | S,R | 297.4 | 298.1 | 19.80 (3) |
| 14-17 | indan-4-yl | R,S and S,R | 309.4 | 310.1 | 5.50 (2) |
| 14-18 | 2,3-dimethyl-phenyl | R,S and S,R | 297.4 | 298.1 | 5.30 (2) |
| 14-19 | 2,3-Dihydro-benzo | R,S and S,R | 327.4 | 328.0 | 4.35 (2) |
| | [1,4]dioxin-5-yl | | | | |
| 14-20 | 2,2-Dimethyl-benzo | R,S and S,R | 341.4 | 342.1 | 5.18 2 |
| | [1,3]dioxol-4-yl | | | | |
| 14-21 | 2-methoxy-4-methyl- | R,S and S,R | 313.4 | 314.1 | 4.76 (2) |
| | phenyl | | | | |

### Example 15

### [4-(2,3-DIMETHYL-PHENOXY)-CHROMAN-3-YLMETHYL]-METHYL-AMINE

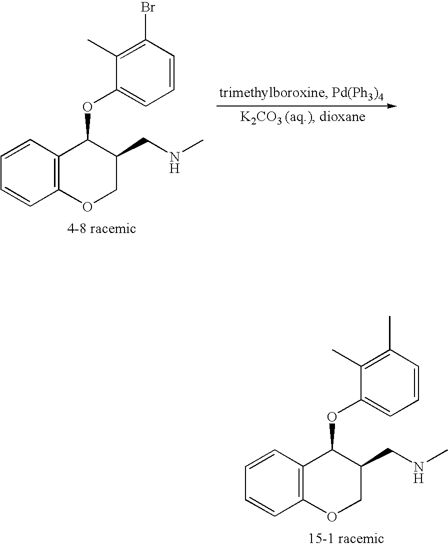 Step 15A:

Compound 4-8 (56 mg, 0.15 mmol), trimethylboroxine (50% in water, 43 mg, 0.17 mmol) and potassium carbonate (64 mg, 0.46 mmol) were taken up in water (50 μL) and dioxane (0.5 mL) and the mixture was degassed with a stream of nitrogen. Palladium tetrakistriphenylphosphine (19 mg, 0.02 mmol) was added and the mixture was heated at 110° C. (oil bath temperature), under an atmosphere of nitrogen overnight. The suspension was partitioned between water and ethyl acetate (20 mL each). The aqueous layer was extracted with ethyl acetate (2×20 mL). The combined organic layers were dried over magnesium sulfate and evaporated. The residue was purified via flash chromatography on silica gel using 2-4% methanol in dichloromethane as eluent to give 15-1(21 mg, 47% ).

¹H NMR (300 MHz, CDCl₃) δ 7.16-7.21 (m, 1H), 7.00-7.11 (m, 2H), 6.92 (dd, J=7.8, 1.5 Hz, 1H), 6.82-6.87 (m, 2H), 6.73 (dt, J=7.8, 1.5 Hz, 1H), 5.34 (d, J=3.0 Hz, 1H), 4.43 (app t, J=11.1 Hz, 1H), 4.32 (ddd, J=10.8, 3.6, 0.6 Hz, 1H), 2.95 (dd, J=12.0, 6.9 Hz, 1H), 2.79 (dd, J=12.0, 7.2 Hz, 1H), 2.46 (s, 3H), 2.43-2.53 (m, 1H), 1.91 (s, 6H). APCI MS m/e: 298.1 ([M+H]⁺)

The enantiomerically pure compounds, 15-2 (R,R enantiomer, APCI MS m/e: 298.1 ([M+H]⁺), t_{R}=5.40 by method 2) and 15-3 (S,S enantiomer, APCI MS m/e: 298.1 ([M+H]⁺) t_{R}=5.32 by method 2) were obtained from 15-1 by chiral preparative HPLC. Starting with compound 4-2 and following the same procedure, [4-(2,4-dimethyl-phenoxy)-chroman-3-ylmethyl]-methyl-amine 15-4 ([M+H]⁺=298.1) was made.

### Example 16

### [4-(2-ETHYL-PHENOXY)-CHROMAN-3-YLMETHYL]-METHYL-AMINE

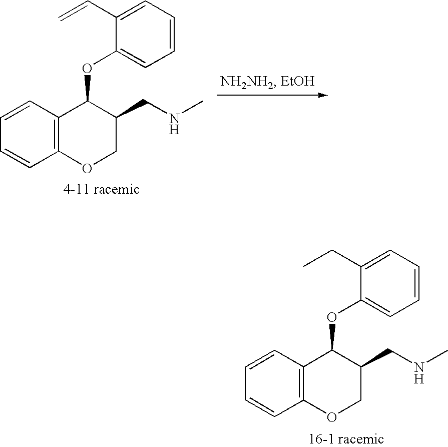 Step 16A:

Example 4-11 (35 mg, 0.12 mmol) was dissolved in ethanol (1 mL) and anhydrous hydrazine (0.1 mL, 3.6 mmol) was added. After two hours at room temperature, the mixture was concentrated in vacuo and the residue purified by flash chromatography on silica using 2-4% methanol in dichloromethane to give example 16-1 (17 mg, 47% yield).

¹H NMR (300 MHz, CDCl₃) δ 7.12-7.21 (m, 4H), 6.93-6.98 (m, 2H), 6.85 (dd, J=8.4, 0.9 Hz, 1H), 6.73 (dt, J=7.5, 1.2 Hz, 1H), 5.43 (d, J=3.6 Hz, 1H), 4.407 (app t, J=10.8 Hz, 1H), 4.32 (dd, J=10.8, 3.6 Hz, 1H), 2.94 (dd, J=12.3, 6.9 Hz, 1H), 2.78 (dd, J=12.0, 6.9 Hz, 1H), 2.46 (s, 3H), 2.37-2.53 (m, 3H), 1.00 (t, J=7.5 Hz, 3H).

APCI MS m/e: 298.1 ([M+H]⁺)

The enantiomerically pure compounds, 16-2 (R,R enantiomer, APCI MS m/e: 298.0 ([M+H]⁺), t_{R}=5.44 by method 2) and 16-3 (S,S enantiomer, APCI MS m/e: 298.1 ([M+H]⁺) t_{R}=5.46 by method 2) were obtained from 16-1 by chiral preparative HPLC.

### Example 17

### 3-METHYL-4-(3-METHYLAMINOMETHYL-CHROMAN-4-YLOXY)-PHENOL

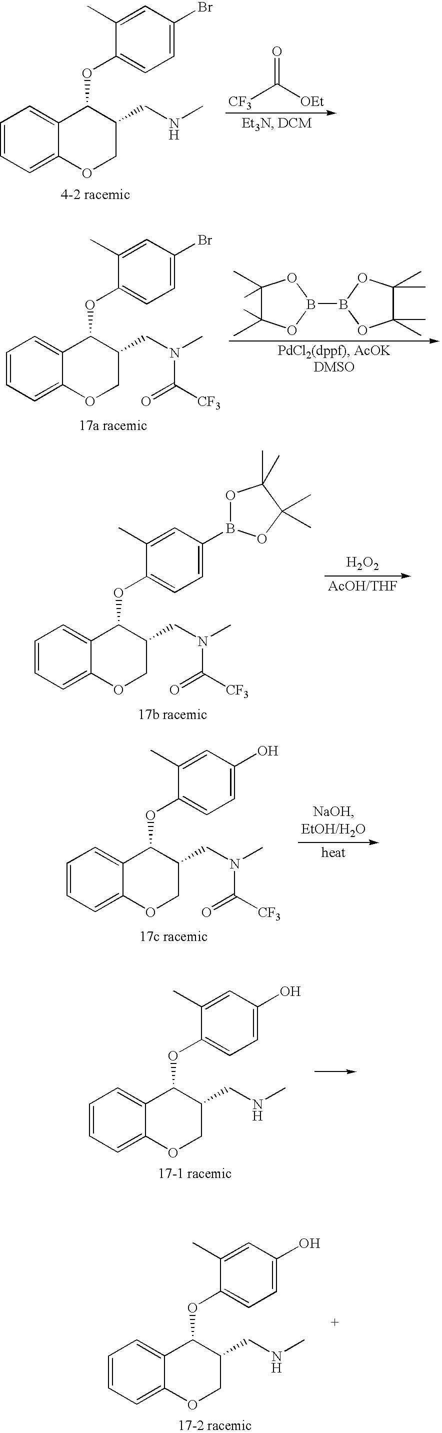 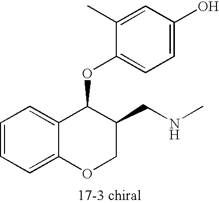 Step 17A:

To a stirred solution of 4-2 (350 mg, 1.0 mmol) in dichloromethane (10 mL) was added triethylamine (0.27 mL, 2.0 mmol) and ethyl trifluoroacetate (0.08 mL, 1.1 mmol). The reaction was allowed to proceed at room temperature. Additional triethylamine (30 equivalents) and ethyl trifluoroacetate (20 equivalents) were added over the course of four days until the reaction was 95% complete as determined by HPLC/MS. The reaction mixture was diluted with dichloromethane (30 mL) and washed with saturated aqueous sodium bicarbonate solution (30 mL). The organic layer was separated and dried over magnesium sulfate. After filtration and concentration, the crude product was purified by flash column chromatography on silica gel using 0-15% ethyl acetate in hexanes as eluent to give 17a as a clear oil (310 mg, 70%).

¹H NMR (300 MHz, CDCl₃) δ 7.17-7.30 (m, 3H), 6.84-6.97 (m, 3H), 6.73 (t, J=6.9 Hz, 1H), 5.20 (d, J=3.0 Hz, 1H), 4.38 (app t, J=10.2 Hz, 1H), 4.21-4.25 (m, 1H), 3.51-3.69 (m, 2H), 2.97 (s, 3H), 2.67 (br s, 1H), 1.94 (s, 3H).

Step 17B:

A solution of 17a (310 mg, 0.68 mmol) in dimethylsulfoxide (5 mL) was degassed with nitrogen for 30 minutes. Bis(pinacolato)diboron (260 mg, 1.02 mmol), potassium acetate (200 mg, 2.03 mmol) and 1,1′-bis(diphenylphosphino)ferrocene palladium (II) chloride (56 mg, 0.068 mmol) were added to this degassed solution. The reaction vessel was sealed and heated at 90° C. for 2.5 hours. After cooling, the mixture was partitioned between ethyl acetate (40 mL) and water (30 mL). The aqueous layer was extracted with ethyl acetate (40 mL) and the combined organic layers were dried over magnesium sulfate. Evaporation produced a residue, which was purified via flash chromatography on silica gel, eluting with 0-20% ethyl acetate in hexanes to give 17b as a white solid (350 mg, 100%).

¹H NMR (300 MHz, CDCl₃) δ 7.65 (d, J=8.1 Hz, 1H), 7.57 (s, 1H), 7.03-7.21 (m, 3H), 6.85 (d, J=8.1 Hz, 1H), 6.72-6.78 (m, 1H), 5.40 (d, J=3.0 Hz, 1H), 4.39 (app t, J=10.5 Hz, 1H), 4.18-4.28 (m, 1H), 3.36-3.58 (m, 2H), 2.95 (s, 3H), 2.61-2.79 (m, 1H), 2.04 (s, 3H), 1.56 (s, 6H), 1.34 (s, 6H).

Step 17C:

To a stirred solution of 17b (343 mg, 0.68 mmol) in tetrahydrofuran (2.4 mL) was added acetic acid (0.092 mL) and hydrogen peroxide (30% in water, 0.14 mL, 1.36 mmol). The reaction mixture was stirred overnight, then was partitioned between dichloromethane and water. The organic layer was dried over magnesium sulfate, filtered and concentrated to afford 17c as a yellow oil (273 mg, 98%).

Step 17D:

A solution of 17c (273 mg, 0.69 mmol) in ethanol (4 mL) was treated with aqueous sodium hydroxide solution (6M, 2 mL) and heated at 80° C. overnight. After cooling to room temperature the mixture was partitioned between dichloromethane (50 mL) and water (20 mL). The aqueous layer was separated and extracted with dichloromethane (50 mL). The combined organic extracts were dried over magnesium sulfate, filtered and concentrated. Purification via flash chromatography on silica gel, eluting with 0.5 to 10% methanol in dichloromethane afforded pure 17-1 (70 mg, 34%).

¹H NMR (300 MHz, CDCl₃) δ 7.18 (ddd, J=8.4, 6.9, 1.5 Hz, 1H), 6.90 (dd, J=8.1, 1.8 Hz, 1H), 0.80-6.84 (m, 2H), 6.73 (ddd, J=7.5, 7.5, 1.5 Hz, 1H), 6.55 (d, J=3.3 Hz, 1H), 6.46 (dd, J=8.7, 3.0 Hz, 1H), 5.21 (d, J=3.6 Hz, 1H), 4.39 (dd, J=10.8 Hz, 9.3 Hz, 1H), 4.27 (dd, J=11.1, 3.0 Hz, 1H), 3.85 (br s, 1H), 3.48 (s, 3H), 3.02 (dd, J=12.3, 7.2 Hz, 1H), 2.82 (dd, J=12.3, 6.6 Hz, 1H), 2.53-2.56 (m, 1H), 2.50 (s, 3H).

APCI MS m/e: 300.0 ([M+H]⁺).

Step 17D:

The enantiomerically pure compounds, 17-2 and 17-3 (30 mg each, 86% recovery), were obtained from racemic 17-1 (70 mg) by chiral preparative HPLC using a Chiralcel OD-H (20×250 cm) column eluting with 85:15 hexanes/ethanol containing 0.1% diethylamine at a flow rate of 15 mL/min.

### Example 18

### C-(4-O-TOLYLOXY-CHROMAN-3-YL)-METHYLAMINE

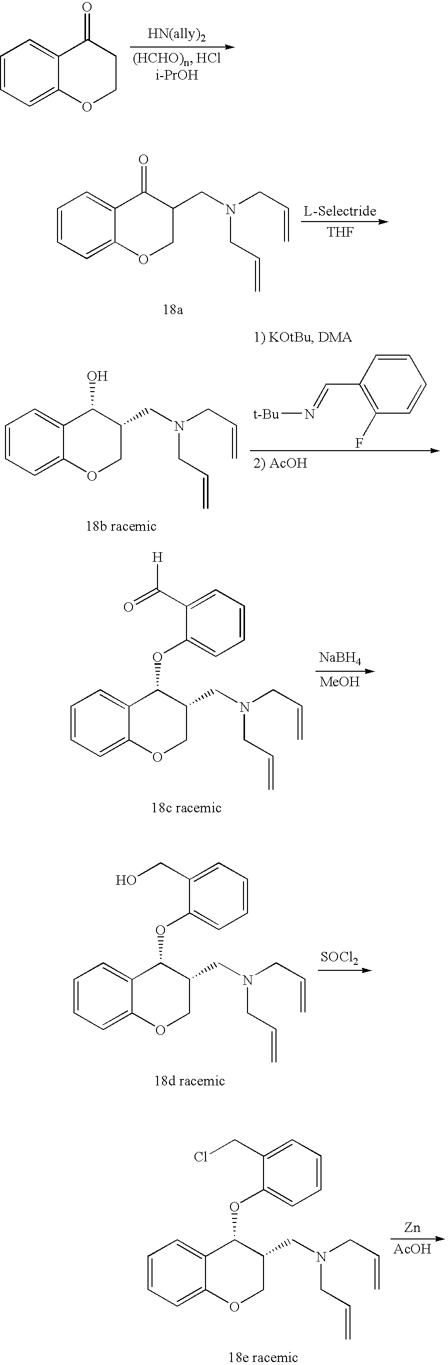 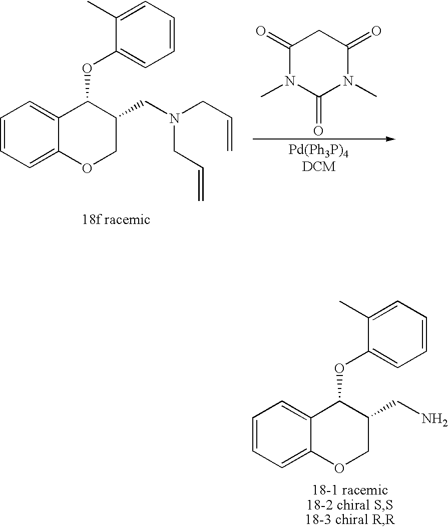 Step 18A:

To a solution of diallylamine (9.8 g, 101 mmol) in 2-propanol (50 mL) was added a solution of hydrochloric acid in diethyl ether (2.0 M, 56 mL, 111 mmol), followed by 4-chromanone (5.0 g, 33.7 mmol) and paraformaldehyde (5.2 g, 168 mmol). The mixture was heated at 80° C. for 2.5 hours. After cooling to room temperature, excess solvent was evaporated and the residue was dissolved in a minimum volume of hot ethanol. The white solid, which precipitated upon cooling, was filtered to yield 6.5 g of 18a. The mother liquor was subjected to a second crystallization to give another 0.72 g of 18a (73% total yield).

¹H NMR (300 MHz, CDCl₃) δ 7.83 (dd, J=1.5, 1.2 Hz, 1H), 7.51 (ddd, J=8.7, 7.5, 1.8 Hz, 1H), 6.97-7.05 (m, 2H), 6.11-6.26 (m, 2H), 5.45-5.63 (m, 4H), 5.16 (dd, J=11.4, 5.4 Hz, 1H), 4.34 (dd, J=11.4, 10.8 Hz, 1H), 3.52-3.91 (m, 6H), 3.00 (dd, J=13.2, 5.1 Hz, 1H). APCI MS m/e: 258.1 ([M+H]⁺).

Step 18B:

To a stirred suspension of 18a (3.6 g, 12.2 mmol) in tetrahydrofuran (30 mL) at −40° C. was added L-Selectride (1.0 M in THF, 25.6 mL) dropwise. After the addition was complete, the reaction was quenched with an aqueous sodium hydroxide solution (1.0 M, 30 mL) at −30° C. The reaction mixture was allowed to warm to room temperature and concentrated under vacuum. The aqueous solution was extracted with ethyl acetate (2×50 mL). The combined organics were dried over magnesium sulfate, filtered and evaporated. The crude product was purified via flash column chromatography on silica gel using 0-50% ethyl acetate in hexanes to give 18b as a yellow oil (2.1 g, 60%). APCI MS m/e: 260.1 ([M+H]⁺).

Step 18C:

To a solution of 18b (1.0 g, 3.86 mmol) in N,N-dimethylacetamide (8 mL) was added potassium t-butoxide (0.65 g, 5.79 mmol) in one portion under an atmosphere of nitrogen. After 30 minutes, a solution of 1-fluoro-2-(t-butylimino)benzene (0.86 g, 4.83 mmol) in N,N-dimethylacetamide (2 mL) was added and the resulting mixture was stirred at room temperature overnight. The reaction mixture was then treated with another portion of potassium t-butoxide (430 mg, 3.86 mmol) and 1-fluoro-2-(t-butylimino)benzene (690 mg, 3.86 mmol). After 5 hours, the reaction mixture was basified by dropwise addition of an aqueous solution of 3M sodium hydroxide. This mixture was extracted with ethyl acetate (2×45 mL). The combined organic layer was extracted with 1M aqueous hydrochloric acid (2×45 mL), which was subsequently basified to pH=10 with 3M aqueous sodium hydroxide. The basic aqueous layer was finally extracted with ethyl acetate (3×50 mL). The combined organic layers were dried over magnesium sulfate, filtered and concentrated under vacuum to give 18c (400 mg, 29%).

¹H NMR(300 MHz, CDCl₃) δ 10.11 (s, 1H), 7.79 (dd, J=8.1, 1.8 Hz, 1H), 7.61 (ddd, J=8.7, 6.9, 1.5 Hz, 1H), 7.43 (d, J=8.4 Hz, 1H), 7.21 (ddd, J=8.7, 7.5, 1.8 Hz, 1H), 7.09 (app t, J=7.2 Hz, 1H), 6.88-6.93 (m, 2H), 6.77 (ddd, J=7.2, 7.2, 1.2 Hz, 1H), 5.69-5.82 (m, 2H), 5.48 (d, J=2.1 Hz, 1H), 5.15 (d, J=1.8 Hz, 1H), 5.05-5.09 (m, 3H), 4.25-4.30 (m, 2H), 3.02-3.18 (m, 4H), 2.74 (dd, J=12.0, 7.5 Hz, 1H), 2.47-2.59 (m, 2H).

APCI MS m/e: 364.1 ([M+H]⁺).

Step 18D:

Aldehyde 18c was reduced to 18d following the procedure for step 7B.

¹H NMR (300 MHz, CDCl₃) δ 7.18-7.31 (m, 3H), 7.11 (dd, J=8.7, 0.9 Hz, 1H), 7.01 (ddd, J=7.5, 7.5, 1.2 Hz, 1H), 6.87 (ddd, J=7.5, 7.5, 1.8 Hz, 1H), 6.73 (ddd, J=7.5, 7.5, 1.2 Hz, 1H), 5.77-5.90 (m, 2H), 5.37 (d, J=2.7 Hz, 1H), 5.10-5.19 (m, 4H), 4.48 (d, J=12.9 Hz, 1H), 4.37 (d, J=12.9 Hz, 1H), 4.25-4.34 (m, 2H), 3.06-3.22 (m, 4H), 2.78-2.86 (m, 2H), 2.47-2.57 (m, 2H). APCI MS m/e: 366.1 ([M+H]⁺).

Step 18E:

Alcohol 18d was converted to chloride 18e following the procedure for step 7C.

¹H NMR (300 MHz, CDCl₃) δ 7.17-7.37 (m, 4H), 6.95-7.02 (m, 2H), 6.88 (dd, J=8.4, 1.5 Hz, 1H), 6.73 (ddd, J=7.2, 7.2, 0.9 Hz, 1H), 5.73-5.87 (m, 2H), 5.45 (s, 1H), 5.07-5.16 (m, 4H), 4.50 (d, J=11.4 Hz, 1H), 4.36-4.43 (m, 1H), 4.25-4.31 (m, 2H), 3.11 (d, J=6.6 Hz, 4H), 2.72-2.77 (m, 1H), 2.49-2.59 (m, 2H). APCI MS m/e: 384.0 ([M+H]⁺).

Step 18F:

Chloride 18e was reduced to 18f following the procedure for step 7D (173 mg, 52% for 3 steps).

¹H NMR (300 MHz, CDCl₃) δ 7.16-7.21 (m, 3H), 7.08 (d, J=7.5 Hz, 1H), 6.85-6.93 (m, 3H), 6.72 (dd, J=7.5 Hz, 1H), 5.73-5.86 (m, 2H), 5.29-5.31 (m, 1H), 5.07-5.16 (m, 4H), 4.38 (app t, J=9.9 Hz, 1H), 4.29 (dd, J=9.9, 3.3 Hz, 1H), 3.06-3.17 (m, 4H), 2.72 (dd, J=12.3, 6.6 Hz, 1H), 2.46-2.58 (m, 2H), 1.95 (s, 3H). APCI MS m/e: 350.1 ([M+H]⁺).

Step 18G:

To a degassed solution of 18f (87 mg, 0.25 mmol) in dichloromethane (1.0 mL) was added 1,3-dimethylbarbituric acid (232 mg, 1.50 mmol) and palladium tetrakistriphenylphosphine (58 mg, 0.05 mmol). The reaction mix was stirred at 35° C. overnight. The mixture was diluted with dichloromethane (25 mL) and washed with 1M aqueous hydrochloric acid (2×15 mL). The aqueous layer was made basic with 3M sodium hydroxide solution and extracted with dichloromethane (2×30 mL). The combined organic extracts were dried over magnesium sulfate, filtered and concentrated. The crude material was purified via column chromatography on silica gel eluting with 0-6% methanol in dichloromethane containing 0.1% triethylamine to give 18-1 (19.4 mg, 29%).

¹H NMR (300 MHz, CDCl₃) δ 7.09-7.22 (m, 4H), 6.97 (dd, J=7.5, 1.8 Hz, 1H), 6.91 (ddd, J=7.2, 7.2, 1.5 Hz, 1H), 6.85 (dd, J=8.4, 0.9 Hz, 1H), 6.75 (ddd, J=7.5, 7.5, 1.5 Hz, 1H), 5.45 (d, J=3.6 Hz, 1H), 4.42 (app t, J=10.5 Hz, 1H0, 4.31 (dd, J=10.5, 3.0 Hz, 1H), 2.86-3.15 (m, 2H), 2.33-2.43 (m, 1H), 2.01 (s, 3H).

Step 18H:

The enantiomerically pure compounds, 18-2 and 18-3 (5 mg each, 42% recovery), were obtained from racemic 18-1 (24 mg) by chiral preparative HPLC using a Chiralcel OJ-H (20×250 cm) column eluting with 65:35 hexanes/ethanol containing 0.1% diethylamine at a flow rate of 15 mL/min. Starting with compound 22c, the following compounds were made by this procedure.

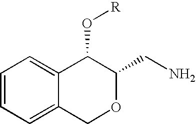

| No. | R | Stereochem. | MW | MH⁺ | t_{R} (method) |
|---|---|---|---|---|---|
| 18-4 | 2-Me-phenyl | S,S and R,R | 269.3 | — | — |
| 18-5 | 2-Me-phenyl | R,R | 269.3 | 270.1 | 6.60 (5) |
| 18-6 | 2-Me-phenyl | S,S | 269.3 | 270.0 | 6.69 (5) |

### EXAMPLE 19

### [4-(2-METHOXYMETHYL-PHENOXY)-CHROMAN-3-YLMETHYL]-METHYL-AMINE

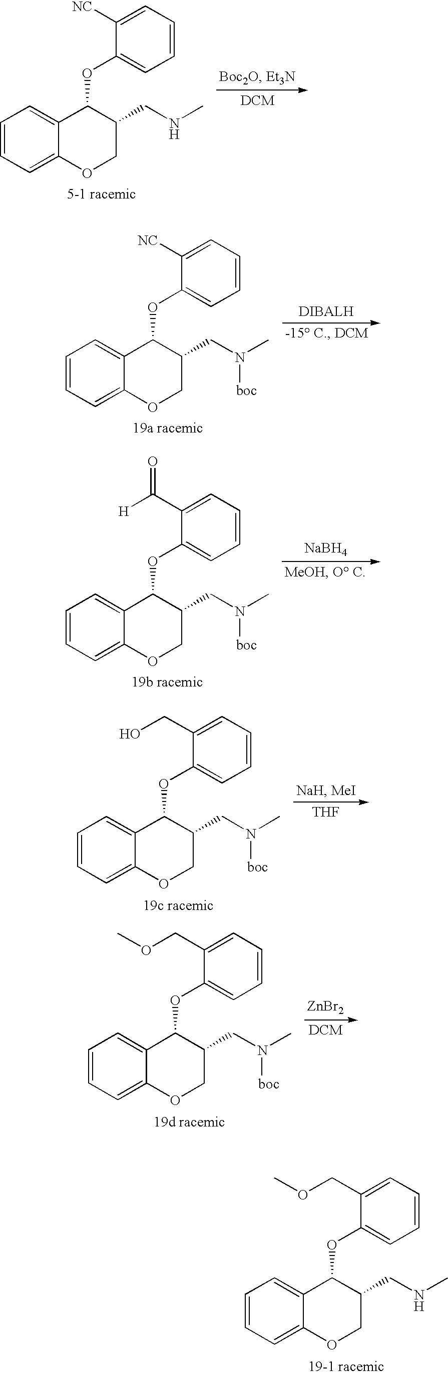 Step 19A:

To a solution of 5-1 (385 mg, 1.3 mmol) and triethylamine (0.73 mL, 5.2 mmol) in dichloromethane (14 mL) was added di-tert-butyldicarbonate (320 mg, 1.4 mmol) as a solution in dichloromethane (10 mL). After 4 days at room temperature, the reaction mixture was diluted with dichloromethane (20 mL) and washed with 0.3M aqueous hydrochloric acid (30 mL). The organic phase was dried over magnesium sulfate, filtered and concentrated to afford 19a as a yellow foam (435 mg, 84%).

APCI MS m/e: 295.0 ([M+H-Boc]⁺).

Step 19B:

A stirred solution of 19a (367 mg, 0.93 mmol) in dichloromethane (7 mL) was chilled to −15° C. under an atmosphere of nitrogen. Diisobutylaluminum hydride (1.0 M in toluene, 0.93 mL) was added to this solution dropwise. After 20 minutes, more diisobutylaluminum hydride (0.93 nL) was added. The reaction mixture was allowed to warm to room temperature and was treated with one last portion of diisobutylaluminum hydride (0.46 mL). The reaction was quenched with 0.3M hydrochloric acid (10 mL) and an aqueous solution of saturated ammonium chloride (2 mL) and stirred at room temperature for an hour. The layers were separated and the aqueous layer was extracted with dichloromethane (2×10 mL). The combined organics were dried over magnesium sulfate, filtered and concentrated under vacuum. The crude product was purified by column chromatography on silica gel eluting with 0-20% ethyl acetate in hexanes to afford 19b as a colorless oil (160 mg, 43%).

¹H NMR (300 MHz, CDCl₃) δ 10.21 (br s, 1H), 7.80 (d, J=7.5 Hz, 1H), 7.61 (ddd, J=8.7, 1.8 Hz, 1H), 7.19-7.31 (m, 2H) 7.11 (app t, J=7.5 Hz, 1H), 6.75-6.94 (m, 3H), 5.45 (br s, 1H), 4.38 (app t, J=10.5 Hz, 1H), 4.28 (dd, J=11.1, 3.9 Hz, 1H), 3.37-3.65 (m, 2H), 2.93 (s, 3H), 2.64-2.83 (m, 1H), 2.93 (s, 9H). APCI MS m/e: 298.0 ([M+H-Boc]⁺).

Step 19C:

To a solution of 19b (60 mg, 0.15 mmol) in methanol (3 mL) at 0° C., was added sodium borohydride (10 mg, 0.16 mmol). The mixture was allowed to warm to room temperature overnight. The reaction mixture was concentrated under vacuum and partitioned between water and ethyl acetate (30 mL each). The aqueous layer was extracted with ethyl acetate (2×30 mL). The combined organic layers were dried over magnesium sulfate, filtered and concentrated to give 19c, which was used without further manipulation. APCI MS m/e: 300.0 ([M+H-Boc]⁺).

Step 19D:

To a suspension of sodium hydride (60% dispersion in oil, 12 mg, 0.3 mmol) in tetrahydrofuran (0.5 mL) was added 19c (0.15 mmol) as a solution in tetrahydrofuran (0.5 mL). After 20 minutes, iodomethane (0.028 mL, 0.45 mmol) was added and the mixture was stirred at room temperature overnight. The reaction mixture was partitioned between water and ethyl acetate (30 mL each). The aqueous layer was extracted with ethyl acetate (30 mL) and the combined organic extracts were dried over magnesium sulfate. Filtration and concentration under vacuum afforded 19d as a colorless oil, which was used without further manipulation. APCI MS m/e: 314.1 ([M+H-Boc]⁺).

Step 19E:

To zinc bromide (68 mg, 0.30 mmol) under nitrogen was added 19d (0.15 mmol) as a solution in dichloromethane (1 mL). The reaction was allowed to proceed under nitrogen at room temperature overnight. The mixture was diluted with dichloromethane (10 mL) and washed with water (2×10 mL). The organic layer was dried over magnesium sulfate, filtered and concentrated under vacuum to afford a brown oil. This crude material was purified via column chromatography on silica gel eluting with 0-6% methanol in dichloromethane to give pure 19-1 (5.8 mg, 12% over 3 steps).

¹H NMR (300 MHz, CDCl₃) δ 7.01-7.40 (m, 6H), 6.79-6.87 (m, 2H), 5.77 (d, J=4.2 Hz, 1H), 4.67 (d, J=12.3 Hz, 1H), 4.43 (dd, J=12.0, 5.4 Hz, 1H), 4.36 (dd, J=12.0, 2.7 Hz, 1H), 4.19 (d, J=12.3 Hz, 1H), 3.27 (s, 3H), 3.14 (d, J=6.9 Hz, 2H), 2.97-3.06 (m, 1H), 2.57 (s, 3H). APCI MS m/e: 314.1 ([M+H]⁺).

### Example 20

### [4-(2-ETHYL-PHENOXY)-ISOCHROMAN-3-YLMETHYL]-METHYL-AMINE

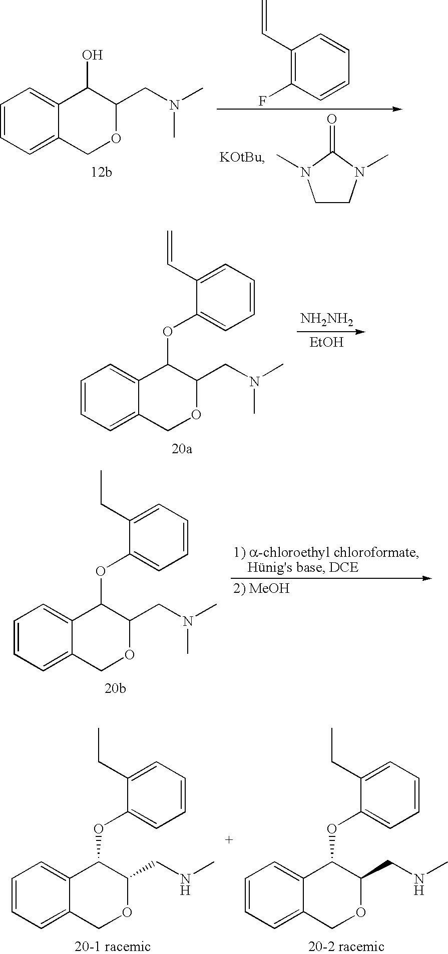 Step 20A:

A mixture of 12b (500 mg, 2.4 mmol), potassium t-butoxide (540 mg, 4.8 mmol) and 2-fluorostyrene (0.86 mL, 7.2 mmol) was suspended in N,N-dimethylimidazolidinone (2.4 mL) and heated to 150° C. via microwave irradiation for ten minutes. The reaction mixture was partitioned between water, brine and ethyl acetate (20 mL each). The aqueous layer was extracted again with ethyl acetate (20 mL) and the combined organic layers were dried over magnesium sulfate. After filtration and concentration the crude product was purified via chromatography on a prep. HPLC column using mass-triggered collection. The combined pure fractions were evaporated and partitioned between dichloromethane and saturated sodium bicarbonate solution. The organic extract was dried over magnesium sulfate and evaporated to give 20a (84 mg, 13%). APCI MS m/e: 310.1 ([M+H]⁺).

Step 20B:

20a was reduced to 20b following the procedure for step 16A. APCI MS m/e: 312.1 ([M+H]⁺).

Step 20C:

Compound 20b was demethylated as described in Step 13D. Cis and trans isomers 20-1 (13 mg, 16% two steps) and 20-2 (10 mg, 12% two steps) were isolated via prep. HPLC chromatography.

20-1: ¹H NMR (300 MHz, CDCl₃) δ 6.92-7.29 (m, 8H), 5.23 (d, J=2.7 Hz, 1H), 5.02 (d, J=15.3 Hz, 1H), 4.84 (d, J=15.0 Hz, 1H), 4.02-4.07 (m, 1H), 3.10 (dd, J=12.3, 8.4 Hz, 1H), 2.90 (dd, J=12.6, 3.9 Hz, 1H), 2.48 (s, 3H), 2.41 (dq, J=7.5, 2.4 Hz, 2H), 0.97 (t, J=7.5 Hz, 3H). APCI MS m/e: 298.1 ([M+H]⁺).

20-2: ¹H NMR (300 MHz, CDCl₃) δ 7.33 (d, J=7.5 Hz, 1H), 7.17-7.28 (m, 5H), 7.11 (d, J=7.8 Hz, 1H), 7.04 (d, J=7.2 Hz, 1H), 6.95 (ddd, J=7.5, 7.5, 1.5 Hz, 1H), 5.44 (d, J=8.4 Hz, 1H), 4.94 (d, J=15.3 Hz, 1H), 4.86 (d, J=15.6 Hz, 1H), 3.95 (ddd, J=9.0, 8.1, 2.7 Hz, 1H), 2.91 (dd, J=12.9, 2.7 Hz, 1H), 2.71 (dd, J=12.3, 8.1 Hz, 1H), 2.64 (q, J=7.5 Hz, 2H), 2.42 (s, 3H), 1.16 (t, J=7.5 Hz, 3H). APCI MS m/e: 298.1 ([M+H]⁺).

The following compounds were made according to this procedure (skipping Step 20B for compounds 20-3 to 20-9):

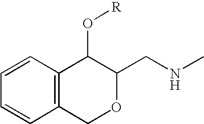

| No. | R | Stereochem | MW | MH⁺ | t_{R} (method) |
|---|---|---|---|---|---|
| 20-1 | 2-ethyl-phenyl | R,R and S,S | 297.4 | 298.1 | 5.13 (2) |
| 20-2 | 2-ethyl-phenyl | R,S and S,R | 297.4 | 298.1 | 5.39 (2) |
| 20-3 | 3-F-2-CH₃-phenyl | R,R and S,S | 301.4 | 301.8 | 8.40 (2) |
| 20-4 | 3-F-2-CH₃-phenyl | R,S and S,R | 301.4 | 302.2 | 9.33 (2) |
| 20-5 | 2-Cl-phenyl | R,R and S,S | 303.8 | 303.8 | 8.01 (2) |
| 20-6 | 2-Cl-phenyl | R,R | 303.8 | 303.6 | 6.77 (5) |
| 20-7 | 2-Cl-phenyl | S,S | 303.8 | 303.8 | 6.74 (5) |
| 20-8 | 2-Cl-phenyl | R,S and S,R | 303.8 | 304.0 | 8.88 (2) |
| 20-9 | 3-Cl-2-CH₃-phenyl | R,S and S,R | 317.8 | 318.0 | 9.98 (2) |

### Example 21

### METHYL-[4-(2-METHYLSULFANYL-PHENOXY)-ISOCHROMAN-3-YLMETHYL]-AMINE

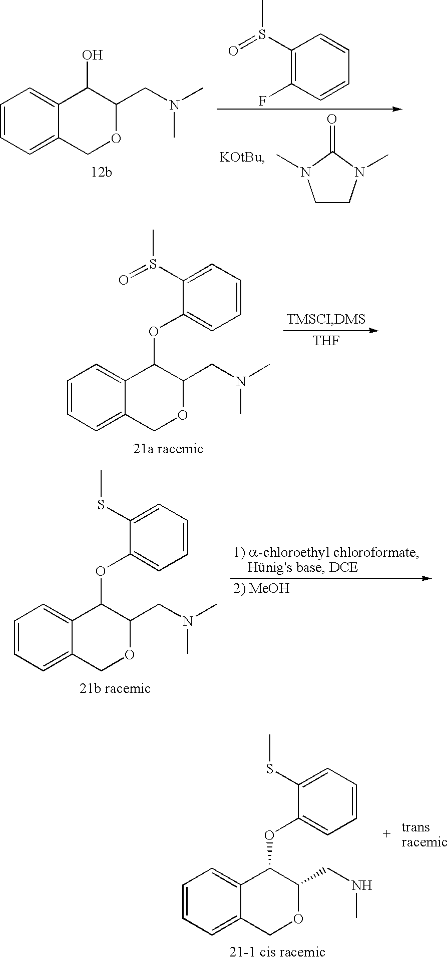 Step 21A:

The procedure for step 20A was followed to convert 12b to 21a (227 mg, 27%).

APCI MS m/e: 346.0 ([M+H]⁺).

Step 21B:

Amine 21a (227 mg, 0.66 mmol) was dissolved in a solution of chlorotrimethylsilane (1.0 M in dichloromethane) and was treated with dimethylsulfide (1.0 mL, 13.2 mmol). The mixture was stirred under an inert atmosphere for 2 hours and was concentrated in vacuo. The residue was partitioned between water and dichloromethane (20 mL each). The aqueous layer was extracted with dichloromethane (20 mL) and the combined organic extracts were dried over magnesium sulfate. Filtration and evaporation gave 21b, which was used without further manipulation.

APCI MS m/e: 330.0 ([M+H]⁺).

Step 21C:

Amine 21b was demethylated as described in Step 13D to give 21-1 (8 mg, 4% two steps). APCI MS m/e: 316.0 ([M+H]⁺

Starting with alcohol 6b and following the procedure for example 21, the racemic mix of cis isomers of methyl-[4-(2-methylsulfanyl-phenoxy)-chroman-3-ylmethyl]-amine 21-2 ([M+H]⁺=316.0) was synthesized.

### Example 22

### 1H-ISOCHROMEN-4(3H)-ONE

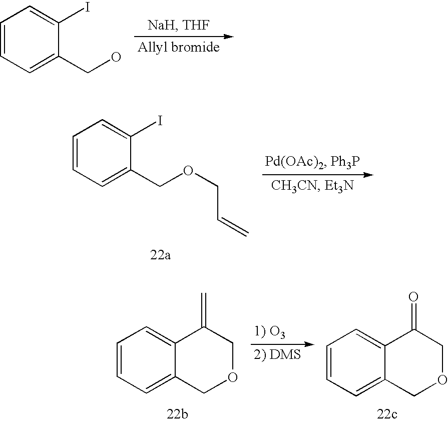 Step 22A:

To the solution of 2-iodobenzyl alcohol (100 g, 0.43 mol) in dry THF (800 mL) at room temperature under a nitrogen atmosphere, sodium hydride (60% dispersion on oil, 20.8 g, 0.52 mol) was added portion-wise. After five minutes, allyl bromide (45 mL, 0.52 mol) was added. The resulting milky suspension was stirred at room temperature overnight. The reaction was carefully quenched with water (200 mL). The two layers were separated and the organic layer was washed with brine (200 mL) and water (200 mL). The organic layer was dried over magnesium sulfate. The excess solvent was evaporated under reduced pressure to give the desired allyl ether 22a as a yellow oil (131 g).

¹H NMR (300 MHz, CDCl₃) δ 7.82 (dd, J=7.8, 1.2 Hz, 1H), 7.44-7.47 (m, 1H), 7.35 (dt, J=7.5, 1.2 Hz, 1H), 6.98 (dt, J=7.5, 1.8 Hz, 1H), 5.93-6.06 (m, 1H), 5.32-5.39 (m, 1H), 5.21-5.26 (m, 1H), 4.50 (s, 2H), 4.11 (dt, J=5.7, 1.2 Hz, 2H).

Step 22B:

The crude allyl ether 22a (252 g, 0.86 mol) was taken up in acetonitrile (1.0 L) and triethylamine (580 mL). The solution was degassed with a stream of nitrogen bubbled through vigorously for 0.5 hour. Palladium acetate (9.6 g, 43 mmol) and triphenyl phosphine (22.5 g, 86 mmol) were added and the solution was refluxed for 2 hours. The mixture was allowed to cool overnight and ether (200 mL) was added. The organics were washed with brine and water and dried over magnesium sulfate. Evaporation gave 22b as a dark syrup, which was used in the next step without further manipulation.

Step 22C:

The alkene 22b was dissolved in 1:1 DCM/MeOH (5 L) containing pyridine (42 mL). The mixture was ozonized in three batches at −78° C., until all starting material was consumed (TLC, about 5 hours per batch). The reaction mixture was then purged thoroughly with nitrogen for half an hour at −78° C. Dimethylsulfide (3 eq.) was added with stirring via an addition funnel over 5 minutes and the cold bath was removed. After warming up to room temperature, the reaction mix was washed twice with water and once with brine. The organic layer was dried over magnesium sulfate and evaporated. Chromatography on a Biotage 150 column using 5% ethyl acetate in hexanes, gave 22c as a yellow oil (49.5 g, 39% over two steps).

¹H NMR (300 MHz, CDCl₃) δ 8.05 (dd, J=8.1, 0.9 Hz, 1H), 7.58 (dt, J=7.5, 1.5 Hz, 1H), 7.43 (dt, J=7.5, 0.9 Hz, 1H), 7.23 (dd, J=7.5, 0.6 Hz, 1H), 4.91 (s, 2H), 4.39 (s, 2H).

### Example 23

### 4-(2-CHLORO-PHENOXY)-THIOCHROMAN-3-YLMETHYL]-METHYL-AMINE

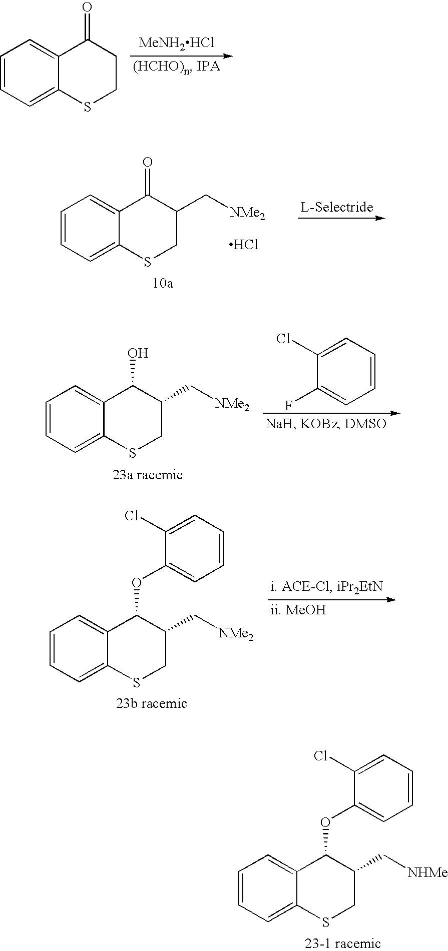 Step 23A:

To thiochroman-4-one (10.0 g, 61 mmol) in 2-propanol (90 mL) was added dimethylamine hydrochloride (9.9 g, 122 mmol), paraformaldehyde (5.5 g, 183 mmol) and hydrochloric acid (2M in diethyl ether, 30.5 mL, 61 mmol). The mixture was heated at 90° C. for 20 h then cooled to ambient temperature. The precipitate was collected by filtration under reduced pressure and was dissolved in the minimum amount of hot ethanol (150 mL). Upon cooling a precipitate formed which was collected by filtration under reduced pressure. The filtrate was concentrated in vacuo to approximately half volume and again the precipitate was collected (this was repeated an additional three times). Amine 10a (10.0 g, 74%) was obtained off-white solid. ¹H NMR (300 MHz, CDCl₃) δ 8.03 (dd, J=8.4 and 1.2 Hz, 1H), 7.40 (td, J=7.2 and 1.2 Hz, 1H), 7.26 (m, 1H), 7.17 (td, J=8.4 and 1.2 Hz, 1H), 3.65-3.86 (m, 2H), 3.25-3.47 (m, 3H), 2.93 (s, 3H), 2.86 (s, 3H). APCI MS m/e: 222.0 ([M+H]⁺).

Step 23B:

To amine 10a (2.0 g, 7.8 mmol) in dichloromethane (40 mL) at −60° C. (acetone/dry ice bath external temperature) was added L-selectride (1 M in tetrahydrofuran, 16.3 mL, 16.3 mmol). The mixture was stirred at that temperature for 1 h then quenched by the addition of 1M aqueous sodium hydroxide (90 mL). The mixture was concentrated in vacuo to remove volatile organics and the residue was extracted with ethyl acetate (100 mL). The ethyl acetate layer was back extracted with 1 M aqueous hydrochloric acid (2×45 mL), the aqueous was basified with 3M aqueous sodium hydroxide solution (universal indicator paper pH 10) and was extracted with ethyl acetate (2×70 mL). The ethyl acetate extracts were dried over magnesium sulfate, filtered and concentrated in vacuo to afford alcohol 23a (90% pure, 1.7 g, 89%) as a colorless oil. ¹H NMR (300 MHz, CDCl₃) δ 7.65 (m, 1H), 7.07-7.16 (m, 3H), 4.80 (d, J=3.3 Hz, 1H), 3.18 (dd, J=12.6 and 4.2 Hz, 1H), 2.57-2.82 (m, 3H), 2.29 (dd, J=11.4 and 3.3 Hz, 1H), 2.22 (s, 6H). APCI MS m/e: 224.0 ([M+H]⁺).

Step 23C:

To a stirred suspension of sodium hydride (60% in mineral oil, 81 mg, 2.0 mmol) in dimethylsulfoxide (2 mL) was added alcohol 23a (300 mg, 1.4 mmol) in dimethylsulfoxide (1.6 mL) under a nitrogen atmosphere. The mixture was stirred at ambient temperature for 15 mins. Potassium benzoate (220 mg, 1.4 mmol) was added and stirring was continued at ambient temperature for a further 15 mins. 2-Chlorofluorobenzene (425 μL, 4.0 mmol) was added and the mixture was stirred at ambient temperature for 20 h. The mixture was diluted with ethyl acetate (40 mL) and washed with 50% aqueous sodium chloride (100 mL). The aqueous layer was extracted with ethyl acetate (40 mL) and the combined organics were back extracted with 1 M hydrochloric acid (2×40 mL). The acidic layer was basified with 3M aqueous sodium hydroxide solution (universal indicator paper pH 10) and was extracted with ethyl acetate (2×50 mL). The ethyl acetate extracts were dried over magnesium sulfate, filtered and concentrated in vacuo to afford amine 23b (90% pure) as a colorless oil which was used in the next step without further purification. ¹H NMR (300 MHz, CDCl₃) δ 7.35 (dd, J=7.8 and 1.8 Hz, 1H), 7.06-7.24 (m, 4H), 6.90-7.05 (m, 3H), 5.53 (d, J=3.0 Hz, 1H), 3.81 (dd, J=13.2 and 8.1 Hz, 1H), 3.35 (dd, J=13.2 and 3.3 Hz, 1H), 3.19 (d, J=6.3 Hz, 2H), 2.85 (m, 1H), 2.70 (s, 6H). APCI MS m/e: 334.0 ([M+H]⁺).

Step 23D:

To a stirred solution of the amine 23b (1.4 mmol) in 1,2-dichloroethane (5 mL) was added N,N-diisopropylethylamine (450 μL, 2.7 mmol) followed by 2-chloroethylchloroformate (440 μL, 4.1 mmol). The mixture was stirred at 40° C. for 1 h then cooled to ambient temperature and quenched with saturated aqueous sodium hydrogen carbonate (20 mL). The mixture was diluted with dichloromethane (30 mL) and the organic layer was washed with saturated aqueous sodium chloride (30 mL), dried over magnesium sulfate, filtered and concentrated in vacuo. The residue was dissolved in methanol (20 mL) and stirred at room temperature for 1 h then concentrated in vacuo. The residue was partitioned between dichloromethane (20 mL) and 1 M aqueous sodium hydroxide (20 mL), the organics were dried over magnesium sulfate, filtered and concentrated in vacuo to afford a yellow oil which was purified by silica gel chromatography eluting with 0 to 5% methanol in dichloromethane. Amine 23-1 (90% pure, 170 mg, 39%) was obtained as a colorless oil. ¹H NMR (300 MHz, CDCl₃) δ 7.32 (dd, J=7.8 and 1.8 Hz, 1H), 7.09-7.20 (m, 3H), 6.84-6.97 (m, 4H), 5.36 (d, J=1.8 Hz, 1H), 3.49 (dd, J=12.3 and 11.1 Hz, 1H), 3.00 (dd, J=12.3 and 5.7 Hz, 1H), 2.97 (dd, J=11.7 and 3.9 Hz, 1H), 2.81 (dd, J=11.7 and 6.0 Hz, 1H), 2.45 (s, 3H), 2.40 (m, 1H). APCI MS m/e: 320.0 ([M+H]⁺).

### Example 24

### 4-(2-CHLORO-PHENOXY)-ISOTHIOCHROMAN-3-YLMETHYL]-METHYL-AMINE

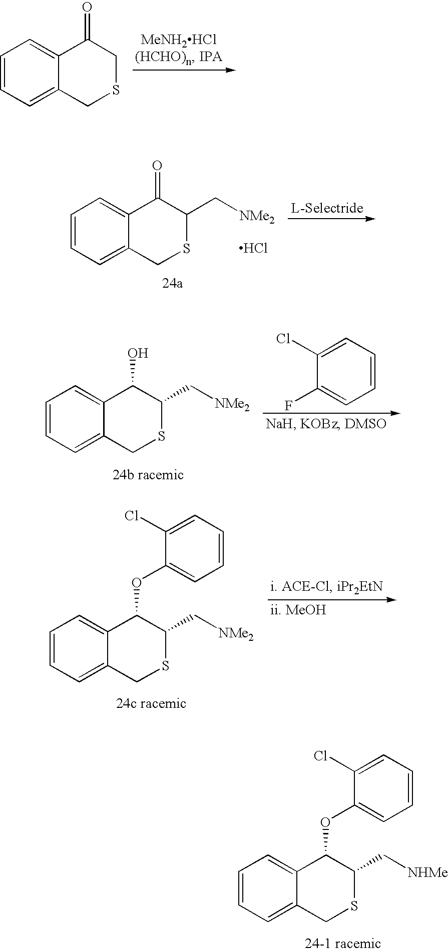 Step 24A:

To thioisochromanone (790 mg, 4.8 mmol) in 2-propanol (7 mL) was added dimethylamine hydrochloride (785 mg, 9.6 mmol), paraformaldehyde (430 mg, 14.4 mmol) and hydrochloric acid (2M in diethyl ether, 2.4 mL, 4.8 mmol). The mixture was heated at 90° C. for 20 h then cooled to ambient temperature. The precipitate was collected by filtration under reduced pressure and was washed with cold 2-propanol (5 mL) and diethyl ether (20 mL) to afford amine 24a (455 mg, 43%) as a brown solid. ¹H NMR (300 MHz, CDCl₃) δ 7.97 (dd, J=7.8 and 1.5 Hz, 1H), 7.47 (td, J=7.8 and 1.5 Hz, 1H), 7.35 (t, J=7.8, 1H), 7.19 (d, J=7.8, 1H), 4.60 (m, 2H), 4.02 (m, 1H), 3.75 (d, J=17.0, 1H), 3.12 (dd, J=13.5 and 6.3 Hz, 1H), 3.01 (s, 3H), 2.93 (s, 3H). APCI MS m/e: 222.0 ([M+H]⁺).

Step 24B:

To amine 24a (390 mg, 1.5 mmol) in dichloromethane (7 mL) at −60° C. (acetone/dry ice bath external temperature) was added L-selectride (1 M in tetrahydrofuran, 3.2 mL, 3.2 mmol). The mixture was stirred at that temperature for 15 min then quenched by the addition of 1M aqueous sodium hydroxide (30 mL). The mixture was diluted with dichloromethane (50 mL), the layers were separated and the aqueous was extracted with dichloromethane (50 mL). The combined organics were dried over magnesium sulfate, filtered and concentrated in vacuo. The residue was purified by silica gel chromatography eluting with 0 to 8% methanol in dichloromethane to afford alcohol 24b (140 mg, 41%) as a pale yellow oil. ¹H NMR (300 MHz, CDCl₃) δ 7.73 (d, J=7.5 Hz, 1H), 7.36 (t, J=7.5 Hz, 1H), 7.26 (t, J=7.5, 1H), 7.13 (d, J=7.5, 1H), 4.92 (d, J=4.5, 1H), 3.79 (d, J=14.1, 1H), 3.72 (dt, J=11.1 and 4.5 Hz, 1H), 3.49 (d, J=14.1, 1H), 2.18 (s, 6H). APCI MS m/e: 224.0 ([M+H]⁺).

Step 24C:

To a stirred suspension of sodium hydride (60% in mineral oil, 38 mg, 0.94 mmol) in dimethylsulfoxide (1.0 mL) was added alcohol 24b (140 mg, 0.63 mmol) in dimethylsulfoxide (0.7 mL) under a nitrogen atmosphere. The mixture was stirred at ambient temperature for 15 mins. Potassium benzoate (100 mg, 0.63 mmol) was added and stirring was continued at ambient temperature for a further 15 mins. 2-Chlorofluorobenzene (200 μL, 1.88 mmol) was added and the mixture was stirred at ambient temperature for 20 h. The mixture was diluted with ethyl acetate (40 mL) and washed with 50% aqueous sodium chloride (50 mL). The organics were dried over magnesium sulfate, filtered and concentrated in vacuo. The residue was purified by silica gel chromatography eluting with 0 to 50% ethyl acetate in hexanes to afford amine 24c as a yellow oil (65 mg, 30%). ¹H NMR (300 MHz, CDCl₃) δ 7.18-7.35 (m, 4H), 7.13 (m, 1H), 7.04 (dd, J=8.4 and 1.2 Hz, 1H), 6.88 (td, J=8.1 and 1.8 Hz, 1H), 5.62 (d, J=3.0 Hz, 1H), 4.34 (d, J=14.0 Hz, 1H), 3.67 (d, J=14.0 Hz, 1H), 3.46 (ddd, J=7.8, 6.3 and 3.0 Hz, 1H), 2.91 (dd, J=12.5 and 6.3 Hz, 1H), 2.51 (dd, J=12.5 and 7.8 Hz, 1H), 2.24 (s, 6H). APCI MS m/e: 334.0 ([M+H]⁺).

Step 24D:

To a stirred solution of the amine 24c (65 mg, 0.2 mmol) in 1,2-dichloroethane (2 mL) was added N,N-diisopropylethylamine (66 μL, 0.4 mmol) followed by 2-chloroethylchloroformate (65 μL, 0.6 mmol). The mixture was stirred at 40° C. for 1 h then cooled to ambient temperature and quenched with saturated aqueous sodium hydrogen carbonate (15 mL). The mixture was diluted with dichloromethane (20 mL) and the organic layer was washed with saturated aqueous sodium chloride (20 mL), dried over magnesium sulfate, filtered and concentrated in vacuo. The residue was dissolved in methanol (10 mL) and stirred at room temperature for 3 h then concentrated in vacuo. The residue was partitioned between dichloromethane (30 mL) and 1M aqueous sodium hydroxide (30 mL), the organics were washed with saturated aqueous sodium chloride (20 mL), dried over magnesium sulfate, filtered and concentrated in vacuo to afford a yellow oil which was purified by silica gel chromatography eluting with 0 to 5% methanol in dichloromethane. Amine 24-1 (25 mg, 40%) was obtained as a pale yellow oil. ¹H NMR (300 MHz, CDCl₃) δ 7.45 (m, 1H), 7.38 (dd, J=7.8 and 1.8 Hz, 1H), 7.11-7.33 (m, 4H), 6.97 (dd, J=7.8 and 1.2 Hz, 1H), 6.92 (dd, J=7.8 and 1.8 Hz, 1H), 5.63 (d, J=3.9 Hz, 1H), 4.05 (d, J=14.0 Hz, 1H), 3.74 (d, J=14.0 Hz, 1H), 3.62 (ddd, J=8.2, 6.0 and 3.9 Hz, 1H), 3.06 (dd, J=12.5 and 6.0 Hz, 1H), 2.54 (dd, J=12.5 and 8.2 Hz, 1H), 2.39 (s, 3H). APCI MS m/e: 320.0 ([M+H]⁺).

### Example 25

### 4-(2-ETHYL-6-FLUORO-PHENOXY)-ISOCHROMAN-3-YLMETHYL]-METHYL-AMINE

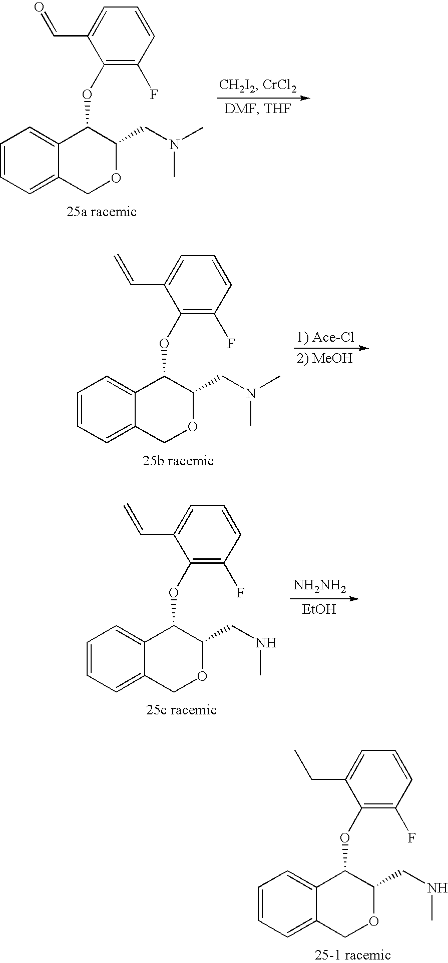 Step 25A:

To a stirred suspension of chromium (II) chloride (590 mg, 4.8 mmol) in tetrahydrofuran (12 mL) was added N,N-dimethylformamide (375 μL, 4.8 mmol). The resultant gelatinous precipitate was sonicated to reform a pale green suspension which was stirred at ambient temperature for 30 min. A solution of aldehyde 25a (prepared according to Step 7A, 200 mg, 0.6 mmol) and diiodomethane (100 μL, 1.2 mmol) in tetrahydrofuran (2 mL) was added to the reaction mixture. The green suspension turned brown and the resultant mixture was stirred at ambient temperature for 16 h. The mixture was diluted with ethyl acetate (50 mL) and washed with 1 M aqueous hydrochloric acid (50 mL). A gelatinous precipitate formed and the biphasic mixture was filtered through celite under reduced pressure. The layers were separated, the organics were dried over magnesium sulfate, filtered and concentrated in vacuo to afford an orange oil which was purified by silica gel chromatography eluting with 0 to 6% methanol in dichloromethane to afford 25b (75% pure, 90 mg, 49%) as a yellow oil. APCI MS m/e: 328.0 ([M+H]⁺).

Step 25B:

Amine 25b was converted to 25c following the procedure for step 23D. ¹H NMR (300 MHz, CDCl₃) δ 6.91-7.26 (7H, m), 6.52 (d, J=7.5 Hz, 1H), 6.37 (dd, J=17.7 and 11.1 Hz, 1H), 5.44 (d, J=17.7 Hz, 1H), 4.89-5.12 (m, 3H), 4.10 (m, 1H), 3.18-3.42 (m, 2H), 2.61 (s, 3H). APCI MS m/e: 314.0 ([M+H]⁺).

Step 25C:

Amine 25c was converted to 25-1 following the procedure for step 16A. ¹H NMR (300 MHz, CDCl₃) δ 7.25 (td, J=8.1 and 1.5 Hz, 1H), 7.70 (d, J=8.1 Hz, 1H), 6.91-6.98 (m, 3H), 6.82 (m, 1H), 6.54 (d, J=7.5 Hz, 1H), 5.08 (d, J=15.3 Hz, 1H), 5.01 (d, J=1.5 Hz, 1H), 4.89 (d, J=15.3 Hz, 1H), 4.01 (ddd, J=8.4, 3.9 and 1.5 Hz, 1H), 3.31 (dd, J=12.5 and 8.4 Hz, 1H), 3.14 (dd, J=12.5 and 3.9 Hz, 1H), 2.57 (s, 3H), 1.90 (m, 2H), 0.79 (t, J=7.5 Hz, 3H). APCI MS m/e: 316.1 ([M+H]⁺).

### Example 26

### [4-(4-HYDROXY-2-METHYL-PHENOXY)-ISOCHROMAN-3-YLMETHYL]-METHYL-AMINE

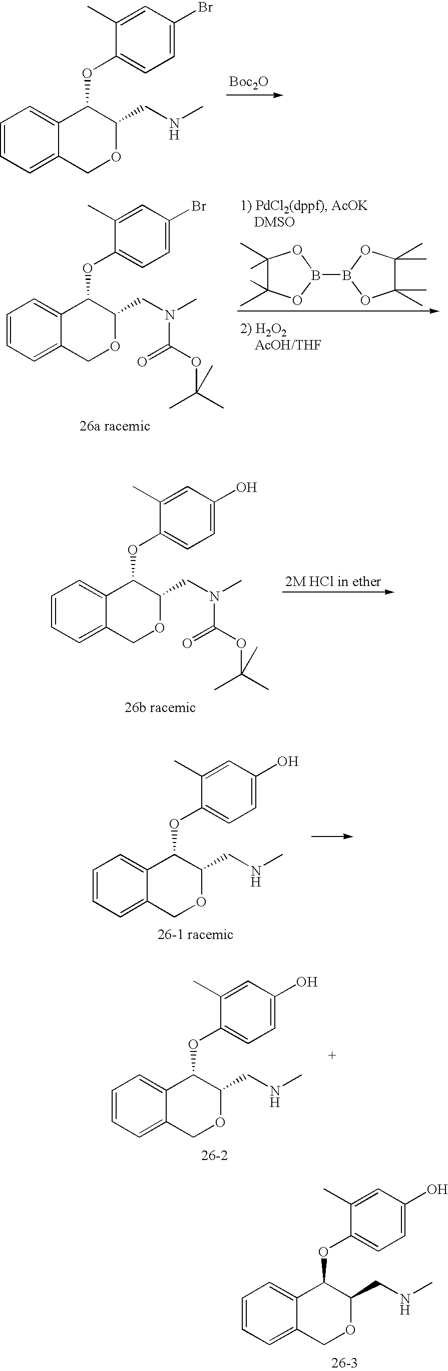 Step 26A:

To [4-(4-Bromo-2-methyl-phenoxy)-isochroman-3-ylmethyl]-methyl-amine (0.22 g, 0.59 mmol, prepared according to Example 12) and triethylamine (0.24 mL, 1.8 mmol) in dichloromethane (5 mL) was added di-tert-butyl-dicarbonate (0.14 g, 0.66 mmol). The mixture was allowed to stir at ambient temperature for 3 h. A further portion of triethylamine (0.12 mL, 0.9 mmol) and di-tert-butyl-dicarbonate (0.07 g, 0.33 mmol) was added and stirring was continued for a further 16 h. The reaction mixture was diluted with dichloromethane (25 mL) and washed with 0.1M aqueous hydrochloric acid (2×10 mL). The combined organics were dried over magnesium sulfate, filtered and concentrated in vacuo. The residue was purified by flash chromatography eluting with 25% ethyl acetate in hexanes to afford 26a (151 mg, 55%) as a pale yellow oil.

APCI MS m/e: 362.0 ([M+H-Boc]⁺).

Step 26B:

Bromide 26a was converted to 26b following the procedures for steps 17B and 17C.

Step 26C:

A solution of 26b (27 mg, 0.07 mmol) in ethanol (1 mL) was treated with hydrochloric acid (2.0M in diethyl ether, 37 μL, 0.07 mmol). The mixture was stirred at ambient temperature for 1.5h. A further portion of hydrochloric acid (2.0M in diethyl ether, 37 μL, 0.07 mmol) was added and the mixture was stirred at ambient temperature for 16 h. A further portion of hydrochloric acid (2.0M in diethyl ether, 105 μL, 0.21 mmol) was added and the mixture was stirred at 50° C. for 16 h. The mixture was concentrated in vacuo, dissolved in ethyl acetate (3 mL) and washed with 1M aqueous sodium hydroxide (3 mL). The aqueous layer was back extracted with ethyl acetate (5×3 mL). The combined organics were dried over magnesium sulfate, filtered and concentrated in vacuo to afford 26-1 (20 mg, quant.) as a colorless oil. APCI MS m/e: 300.1 ([M+H]⁺).

The enantiomerically pure compounds 26-2 and 26-3 were obtained from racemic 26-1 by chiral preparative HPLC using a Chiralcel OJ-H (20×250 cm) column eluting with 85:15 hexanes/ethanol containing 0.1% diethylamine at a flow rate of 15 mL/min.

### Example 27

### INHIBITION OF RADIOLIGAND TRANSPORT BY HEK293 CELLS EXPRESSING HUMAN NOREPINEPHRINE, DOPAMINE, OR SEROTONIN TRANSPORTERS

The norepinephrine, dopamine, and serotonin transporters were individually expressed in stably transfected HEK293 cell lines and grown in Dulbecco's Modified Eagles Medium (DMEM) (Cellgro, 15-013-CV) with the following supplements: 1% HEPES (Cellgro, MT 25-060-Cl); 1% L-glutamine (Cellgro, MT 25-005-Cl); 1% sodium pyruvate (Cellgro, MT 25-000Cl); 1% Pen/Strep (Cellgro, MT 30-001-Cl); 10% heat-inactivated fetal bovine serum (FBS) (Hyclone, Logan, Utah); 250 μg/ml G418 (Cellgro, 61-234-RG).

The day before the assay, solid white 96-well TC-treated sterile plates (Costar, 3917) that had been coated with 0.01% poly-D-lysine (Sigma, P6407) and 0.01% collagen (BD Biosciences, 354236) were seeded with cells at a density of 20,000 cells/well. The cells were allowed to attach overnight in a 37° C. incubator (7.5% CO₂). On the day of the assay, media was removed, and cells were washed with phosphate buffered saline. The cells were then incubated at room temperature for 20 minutes with varying concentrations of competing ligand in a total volume of 150 μl transport buffer (20 mM HEPES, 122 mM NaCl, 3 mM KCl, 1.3 mM CaCl₂, 1.2 mM KH₂PO₄, 0.4 mM MgSO₄, 1 mM ascorbic acid, 0.1 mM pargyline, 0.1 mM tropolone). Radioligand was then added to the cells for a total volume of 200 μl, and cells were incubated at room temperature for an additional 20 minutes. (Levo-[ring-2,5,6-³H]Norepinephrine (52 Ci/mmol, PerkinElmer, NET-678) was used for NET, 3,4-[ring-2,5,6-³H]Dihydroxyphenylethylamine (50 Ci/mmol, PerkinElmer, NET-673) was used for DAT, and [alpha, beta-³H(N)]5-Hydroxytryptamine (30 Ci/mmol, PerkinElmer, NET-498) was used for SERT.) After incubation, the transport buffer was quickly aspirated from the plates, and the cells were washed twice with 4° C. wash buffer (20 mM HEPES, 280 mM D-mannitol, 5.4 mM KCl, 1.8 mM CaCl₂, 0.8 mM MgSO₄, 1 mM ascorbic acid, 0.1 mM pargyline, 0.1 mM tropolone). Cells were treated with 50 μl 5% sodium dodecyl sulfate solution (Sigma, L4522) and 200 μl Microscint scintillation fluid. Plates were shaken vigorously overnight before monitoring radioligand in a TopCount-NXT (Packard) microplate scintillation counter. Data were analyzed by nonlinear, least-squares curve fitting algorithms using ActivityBase (IDBS, Guildford, Surrey, UK).

### Example 28

### RADIOLIGAND BINDING TO HUMAN NET TRANSPORTER EXPRESSED IN MAMMALIAN CELL LINES

Crude membranes were prepared by differential centrifugation from HEK293 cells stably transfected with the human norepinephrine transporter. Membranes (3 μg of protein) were incubated for 2 hours with 1.5 nM [³H]Nisoxetine (86 Ci/mmol, PerkinElmer, NET-1084) in the presence of varying concentrations of competing ligand. Non-specific binding was determined in the presence of excess (1 μM) desipramine. Reactions were terminated by rapid vacuum filtration using a Packard 96-well cell harvester over PEI soaked (1%) (Sigma, P3143) GF/C membrane filter plates (Packard, 6005174). The filter plates were then washed with 600 μl phosphate buffered saline containing 0.01% (v/v) Triton-X100 and dried under forced air fans. Microscint scintillation fluid was added to each well before monitoring bound radioligand in a TopCount-NXT (Packard) microplate scintillation counter. Binding data were analyzed by nonlinear, least-squares curve fitting algorithms using ActivityBase (IDBS, Guildford, Surrey, UK).

### Example 29

### FORMALIN FLINCH ASSAY

The formalin test is conducted using the Automated Nociception Analyzer (Department of Anesthesiology, University of California, San Diego, Yaksh et al, 2001). One hour prior to testing, a metal band is glued to a rat's left hind paw. The animal is then put in a testing chamber. Animals are dosed with compound orally at volumes equal to or less than 10 mg/ml with either vehicle (5% Cremophor® in milliQ water) or active compounds (1-100 mg/kg) one hour prior to formalin injection. As a positive control, rats are dosed with ethosuximide at 600 mg/kg orally. Animals are injected with 50 μl of 5% formalin solution (20-fold dilution of a 37% stock from Fisher Chemicals) subcutaneously on dorsal surface of the left hind paw. The number of flinches is recorded for each minute for one hour by detecting the movement of a metal paw band with a localized low strength sinusoidal electromagnetic field. Drug effects are analyzed by a one-way ANOVA on each phase {Phase I (0-9 minutes), Phase IIA (10-40 minutes), and Phase IIB (41-60 minutes)}. Significant effects are analyzed by Dunnett's post hoc comparison to vehicle.

### Example 30

### SPINAL NERVE LIGATION ASSAY

Neuropathy is induced by Spinal Nerve Ligation (SNL) surgery (Kim and Chung, 1992). Briefly, in rats, the left L5 and L6 spinal neurons distal to the dorsal root ganglion are tightly ligated with 6-0 silk suture. At 4-12 weeks post-surgery, the rats are tested for mechanical hyperalgesia using the pin prick method (Koch et al, 1996). The length of time the paw is held off the grid-floor is measured with a computer program Xnote Stopwatch ver 1.4. Zero seconds is assigned when there is no paw withdrawal. The baseline score is determined from the average of five trials. Baselines are counterbalanced for assignments into treatment groups. Animals are dosed with compound orally at volumes equal to or less than 10 mg/ml with either vehicle (5% Cremophor® in milliQ water) or active compounds (1-100 mg/kg) one hour prior to assessment of withdrawal in response to the pin prick. Drug effects are analyzed by a two-way ANOVA with treatment and time as variables. Significant effects are analyzed by Dunnett's post hoc comparison.

Kim S H, Chung J M., An experimental model for peripheral neuropathy produced by segmental spinal nerve ligation in the rat., Pain. 1992 September;50(3):355-63.

Koch B D, Faurot G F, McGuirk J R, Clarke D E, Hunter J C., Modulation of mechano-hyperalgesia by clinically effective analgesics in rats with a peripheral mononeuropathy. Analgesia. 1996; Vol 2:157-164.

Yaksh T L, Ozaki G, McCumber D, Rathbun M, Svensson C, Malkmus S, Yaksh M C., An automated flinch detecting system for use in the formalin nociceptive bioassay. J Appl Physiol. 2001 June;90(6):2386-402.

### Example 31

### MICRODIALYSIS PROCEDURE FOR THE DETERMINATION OF MONOAMINE LEVELS

Surgery: Animals were anaesthetized with isoflorane, and a servo-controlled heating pad maintained body temperature throughout the surgery. Animals were placed in a stereotaxic instrument and an incision was made down the mid-line over the skullcap. Miniature bone screws were inserted individually into the occipital, parietal and frontal skull plates. Two small holes (1.8 mm diameter) were drilled with micro-trephines for stereotaxic insertion of guide cannulae, one in the left frontal plate (3.1 mm anterior and 1.2 mm lateral to bregma) and a second in the right parietal plate (0.5 mm posterior and 4.4 mm lateral to bregma). Guide cannulae were lowered into the brain at a rate of 0.2 mm/min and at an angle of 5° to the following depths: 2.0 mm (left frontal cannula) and 3.0 mm (right striatal cannula). The dialysis membranes of the microdialysis probes have a 3.0 mm length and extend 3.0 mm past the ends of the implanted cannulae so the final depths of inserted probes were 3.0 mm and 6.0 mm for the PFC and striatal probes respectively. The sampled brain regions correspond to (1) left prefrontal cortex (PFC), including mainly anterior cingulate and prelimbic cortices, and (2) right striatum (caudate-putamen) mainly, but also including to a small degree in some animals, lateral globus pallidus. Cannulae were secured with dental cement to the skull and bone screws. The skin incision was closed with 4-0 suture and Vetbond (3-M). Animals received immediate post-operative care and were allowed one full week to recover from surgery. Animals were housed in 12:12 light-dark room (lights off at 7AM).

Microdialysis procedure: After a 1-week recovery, animals were placed in individual Raturn bowls for microdialysis sampling (Bioanalytical Systems, Inc., West Lafayette, Ind.). The capped stylets that cover the cannulae and maintain their patency were removed, and microdialysis probes were inserted manually at a slow rate. Probe membranes protruded 3.0 mm from the cannula tips and sampled extracellular fluid over this entire 3.0 mm length. The input tube of each microdialysis probe was connected to a syringe pump (CMA/102, CMA Microdialysis, North Chelmsford, Mass.) that delivered artificial cerebrospinal fluid (αCSF). αCSF had the following composition: 154.7 mM Na⁺, 2.9mM K⁺, 1.1 mM Ca²⁺, 0.82 mM Mg²⁺, 132.49 mM Cl⁻ (pH 7.4). The output tubes of each probe were connected to a refrigerated fraction collection system (Honeycomb, Bioanalytical Systems, Inc.). Animals were allowed 14-16 hrs to recover from probe insertion and to habituate to the bowl and tether. Probes were perfused over this time period at a slow rate of 0.2 μL/min. On the following morning, pump perfusion rates were increased to 1.1 μL/min at the time of lights off (7 AM). Dialysate sampling began 1 hr later. Individual samples were collected over a 30 min time period. After 1.5 hrs of baseline sampling (3 samples), either vehicle (5% Cremophor in MillQ water) or NBI compound, which was prepared in vehicle solution, was administered orally at doses of either 1, 3 or 10 mg/kg and at a dose volume of 5 mL/kg. Sampling was continued for 6 hrs after dosing (12 post-dose samples). At the end of the study, sampling carousels were removed and transported to the HPLC-electrochemical detection system. Animals were euthanized with CO₂ and decapitation. Brains were immediately removed and prepared for histology by inserting the brains briefly (1 min) into −40° C. 2-methylbutane. Brain areas of interest (left frontal region, ˜2-4 mm rostral to bregma, and right midline region, ˜0-2 mm caudal to bregma) were sectioned coronally with a cryostat and sections were prepared for histological examination of probe placement and depth.

HPLC-EC detection and analysis: Monoamine levels in microdialysis samples were measured by HPLC-electrochemical detection (EC). 27 uL was withdrawn from individual microdialysis samples and injected onto an HPLC-EC system consisting of a pulse-free pump (Model 582 Solvent Delivery System; ESA Instruments, Chelmsford, Mass.), a 2 mm×15 cm, 3 um particle size LC column (70-4129; ESA Instruments), and an electrochemical detector (Coulochem III; ESA Instruments). The mobile phase consisted of 0.05 M citrate, 1.00 mM OSA, 0.1 mM EDTA, 6.5% methanol, and pH=4.85, and resulted in retention times of approximately 4″, 12″ and 34″ for NE, DA and 5-HT, respectively. The optimal detection settings for the detector were the following: working electrode=+300 mV, reference electrode #1=+200 mV, reference electrode #2=−120 mV, signal filter=0.1 Hz, range=1.0 nA. Chromatograms were analyzed manually off-line (EZChrome Elite software; Agilent Technologies, Pleasantown, Calif.) to determine peak areas for NE (norepinephrine), DA (dopamine) and 5-HT. Daily analysis of monoamine standards showed the quantitative limits for NE, DA and 5-HT was approximately 0.2-0.5 pg per 27 uL of sample. Peak values for each sample were normalized to the mean peak value of the first three baseline samples for each monoamine using Micosoft Excel. Normalized data were imported into GraphPad Prism (GraphPad Software, San Diego, Calif.) for graphical analysis and to test for significant differences between treatment groups using repeated measures, two-way ANOVA for treatment (different dose groups: vehicle, 1, 3 and 10 mg/kg) and time (15 time points). If significant interaction effects were observed, pair-wise post-hoc group comparisons were made using Tukey's least significant difference procedure to test for significant effects of treatment at each time point.

It will be appreciated that, although specific embodiments of the invention have been described herein for purposes of illustration, various modifications may be made without departing from the spirit and scope of the invention. Accordingly, the invention is not limited except as by the appended claims.

We claim:

1. A compound having the following structure: 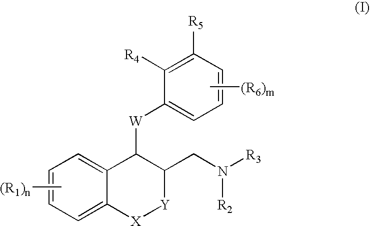
or a stereoisomer, prodrug or pharmaceutically acceptable salt, ester or solvate thereof,
wherein:
W is O or S;
X—Y is —O—, —OCH₂—, —CH₂O—, —SCH₂—, or —CH₂S—;
R₁ at each occurrence is independently halo, CN, CF₃, OH, lower alkyl, lower alkoxy, or lower thioalkyl;
R₂, R₃ are independently H, lower alkyl, or substituted lower alkyl;
R₄, R₅ are independently H, halo, CN, CF₃, OH, NO₂, lower alkyl, substituted lower alkyl, lower alkoxy, substituted lower alkoxy, lower thioalkyl, or substituted lower-thioalkyl;
or R₄ and the carbon to which it is attached taken together with R₅ and the carbon to which it is attached form a 5-6 member carbocycle or a 5-6 member heterocycle where the carbocycle or heterocycle is substituted with 0-4 R₇;
R₆, R₇ are at each occurrence independently H, halo, CN, CF₃, OH, lower alkyl, substituted lower alkyl, lower alkoxy, or lower thioalkyl;
m is 0, 1, 2, or 3; and
n is 0, 1, 2 or 3.

2. The compound of claim 1 wherein —X—Y— is —OCH₂—.

3. The compound of claim 1 wherein —X—Y— is —CH₂O—.

4. The compound of claim 1 wherein —X—Y— is —SCH₂—.

5. The compound of claim 1 wherein —X—Y— is —CH₂S—.

6. The compound of claim 1 wherein W is O.

7. The compound of claim 1 wherein W is S.

8. The compound of claim 1 wherein R₂ is H and R₃ is lower alkyl.

9. The compound of claim 8 wherein W is O.

10. The compound of claim 9 wherein —X—Y— is —CH₂O—.

11. The compound of claim 9 wherein —X—Y— is —OCH₂—.

12. The compound of claim 10 wherein R₄ and R₅ are independently H, halo, CN, CF₃, OH, NO₂, lower alkyl, substituted lower alkyl, lower alkoxy, substituted lower alkoxy, lower thioalkyl, or substituted lower thioalkyl.

13. The compound of claim 11 wherein R₄ and R₅ are independently H, halo, CN, CF₃, OH, NO₂, lower alkyl, substituted lower alkyl, lower alkoxy, substituted lower alkoxy, lower thioalkyl, or substituted lower thioalkyl.

14. The compound of claim 10 wherein R₄ and the carbon to which it is attached taken together with R₅ and the carbon to which it is attached form a 5-6 member carbocycle or a 5-6 member heterocycle where the carbocycle or heterocycle is substituted with 0-4 R₇.

15. The compound of claim 11 wherein R₄ and the carbon to which it is attached taken together with R₅ and the carbon to which it is attached form a 5-6 member carbocycle or a 5-6 member heterocycle where the carbocycle or heterocycle is substituted with 0-4 R₇.

16. The compound of claim 1 wherein R₂ and R₃ are H.

17. A pharmaceutical composition comprising a compound of claim 1 and a pharmaceutically acceptable carrier or diluent.

18. A method of treating a disorder of the central or peripheral nervous system in a subject in need thereof comprising administering to the subject an effective amount of the pharmaceutical composition of claim 17.

19. The method of claim 18 wherein the disorder is depression, anxiety, pain, urinary incontinence, fibromyalgia, or attention deficit hyperactivity disorder (ADHD).

20. The method of claim 19 wherein the disorder is neuropathic pain or fibromyalgia.
